SEQUENCE LISTING (13 sequences)

SEQ ID NO: 1 - DNA, 867 bp, Homo sapiens
atgcagcggaggtgggtcttcgtgctgctcgacgtgctgtgcttactggtcgcctccctgcccttcgctatcctgacgctggtgaacgccccgtacaagcgaggattttactgcggggatgactccatccggtacccctaccgtccagataccatcacccacgggctcatggctggggtcaccatcacggccaccgtcatccttgtctcggccggggaagcctacctggtgtacacagaccggctctattctcgctcggacttcaacaactacgtggctgctgtatacaaggtgctggggaccttcctgtttggggctgccgtgagccagtctctgacagacctggccaagtacatgattgggcgtctgaggcccaacttcctagccgtctgcgaccccgactggagccgggtcaactgctcggtctatgtgcagctggagaaggtgtgcaggggaaaccctgctgatgtcaccgaggccaggttgtctttctactcgggacactcttcctttgggatgtactgcatggtgttcttggcgctgtatgtgcaggcacgactctgttggaagtgggcacggctgctgcgacccacagtccagttcttcctggtggcctttgccctctacgtgggctacacccgcgtgtctgattacaaacaccactggagcgatgtccttgttggcctcctgcagggggcactggtggctgccctcactgtctgctacatctcagacttcttcaaagcccgacccccacagcactgtctgaaggaggaggagctggaacggaagcccagcctgtcactgacgttgaccctgggcgaggctgaccacaaccactatggatacccgcactcctcctcctga

SEQ ID NO: 2 - DNA, 459 bp, Homo sapiens
atgcagcggaggtgggtcttcgtgctgctcgacgtgctgtgcttactggtcgcctccctgcccttcgctatcctgacgctggtgaacgccccgtacaagcgaggattttactgcggggatgactccatccggtacccctaccgtccagataccatcacccacgggctcatggctggggtcaccatcacggccaccgtcatccttgtctcggccggggaagcctacctggtgtacacagaccggctctattctcgctcggacttcaacaactacgtggctgctgtatacaaggttgtctttctactcgggacactcttcctttgggatgtactgcatggtgttcttggcgctgtatgtgcaggcacgactctgttggaagtgggcacggctgctgcgacccacagtccagttcttcctggtggcctttgccctctacgtgggctacacccgcgtgtctga

SEQ ID NO: 3 - DNA, 699 bp, Homo sapiens
atggctggggtcaccatcacggccaccgtcatccttgtctcggccggggaagcctacctggtgtacacagaccggctctattctcgctcggacttcaacaactacgtggctgctgtatacaaggtgctggggaccttcctgtttggggctgccgtgagccagtctctgacagacctggccaagtacatgattgggcgtctgaggcccaacttcctagccgtctgcgaccccgactggagccgggtcaactgctcggtctatgtgcagctggagaaggtgtgcaggggaaaccctgctgatgtcaccgaggccaggttgtctttctactcgggacactcttcctttgggatgtactgcatggtgttcttggcgctgtatgtgcaggcacgactctgttggaagtgggcacggctgctgcgacccacagtccagttcttcctggtggcctttgccctctacgtgggctacacccgcgtgtctgattacaaacaccactggagcgatgtccttgttggcctcctgcagggggcactggtggctgccctcactgtctgctacatctcagacttcttcaaagcccgacccccacagcactgtctgaaggaggaggagctggaacggaagcccagcctgtcactgacgttgaccctgggcgaggctgaccacaaccactatggatacccgcactcctcctcctga

SEQ ID NO: 4 - DNA, 930 bp, Homo sapiens
atgggggtcgcgagaggcccggggagccggggccagcatcccccgccccggcagcaggaagtctgtgcggaggggccgcgcgcgcgcctccatcccgccccgcctggcctgggagcctccctgcccttcgctatcctgacgctggtgaacgccccgtacaagcgaggattttactgcggggatgactccatccggtacccctaccgtccagataccatcacccacgggctcatggctggggtcaccatcacggccaccgtcatccttgtctcggccggggaagcctacctggtgtacacagaccggctctattctcgctcggacttcaacaactacgtggctgctgtatacaaggtgctggggaccttcctgtttggggctgccgtgagccagtctctgacagacctggccaagtacatgattgggcgtctgaggcccaacttcctagccgtctgcgaccccgactggagccgggtcaactgctcggtctatgtgcagctggagaaggtgtgcaggggaaaccctgctgatgtcaccgaggccaggttgtctttctactcgggacactcttcctttgggatgtactgcatggtgttcttggcgctgtatgtgcaggcacgactctgttggaagtgggcacggctgctgcgacccacagtccagttcttcctggtggcctttgccctctacgtgggctacacccgcgtgtctgattacaaacaccactggagcgatgtccttgttggcctcctgcagggggcactggtggctgccctcactgtctgctacatctcagacttcttcaaagcccgacccccacagcactgtctgaaggaggaggagctggaacggaagcccagcctgtcactgacgttgaccctgggcgaggctgaccacaaccactatggatacccgcactcctcctcctga

SEQ ID NO: 5 - protein, 288 aa, Homo sapiens
MQRRWVFVLLDVLCLLVASLPFAILTLVNAPYKRGFYCGDDSIRYPYRPDTITHGLMAGVTITATVILVSAGEAYLVYTDRLYSRSDFNNYVAAVYKVLGTFLFGAAVSQSLTDLAKYMIGRLRPNFLAVCDPDWSRVNCSVYVQLEKVCRGNPADVTEARLSFYSGHSSFGMYCMVFLALYVQARLCWKWARLLRPTVQFFLVAFALYVGYTRVSDYKHHWSDVLVGLLQGALVAALTVCYISDFFKARPPQHCLKEEELERKPSLSLTLTLGEADHNHYGYPHSSS

SEQ ID NO: 6 - protein, 152 aa, Homo sapiens
MQRRWVFVLLDVLCLLVASLPFAILTLVNAPYKRGFYCGDDSIRYPYRPDTITHGLMAGVTITATVILVSAGEAYLVYTDRLYSRSDFNNYVAAVYKVVFLLGTLFLWDVLHGVLGAVCAGTTLLEVGTAAATHSPVLPGGLCPLRGLHPRV

SEQ ID NO: 7 - protein, 232 aa, Homo sapiens
MAGVTITATVILVSAGEAYLVYTDRLYSRSDFNNYVAAVYKVLGTFLFGAAVSQSLTDLAKYMIGRLRPNFLAVCDPDWSRVNCSVYVQLEKVCRGNPADVTEARLSFYSGHSSFGMYCMVFLALYVQARLCWKWARLLRPTVQFFLVAFALYVGYTRVSDYKHHWSDVLVGLLQGALVAALTVCYISDFFKARPPQHCLKEEELERKPSLSLTLTLGEADHNHYGYPHSSS

SEQ ID NO: 8 - protein, 309 aa, Homo sapiens
MGVARGPGSRGQHPPPRQQEVCAEGPRARLHPAPPGLGASLPFAILTLVNAPYKRGFYCGDDSIRYPYRPDTITHGLMAGVTITATVILVSAGEAYLVYTDRLYSRSDFNNYVAAVYKVLGTFLFGAAVSQSLTDLAKYMIGRLRPNFLAVCDPDWSRVNCSVYVQLEKVCRGNPADVTEARLSFYSGHSSFGMYCMVFLALYVQARLCWKWARLLRPTVQFFLVAFALYVGYTRVSDYKHHWSDVLVGLLQGALVAALTVCYISDFFKARPPQHCLKEEELERKPSLSLTLTLGEADHNHYGYPHSSS

SEQ ID NO: 9 - protein, 142 aa, Homo sapiens
AVSQSLTDLAKYMIGRLRPNFLAVCDPDWSRVNCSVYVQLEKVCRGNPADVTEARLSFYSGHSSFGMYCMVFLALYVQARLCWKWARLLRPTVQFFLVAFALYVGYTRVSDYKHHWSDVLVGLLQGALVAALTVCYISDFFK

SEQ ID NO: 10 - DNA, 1269 bp, Homo sapiens
gcgacgggacgcgctgggaccggcgtcgggggtcgcggggaccatgcagcggaggtgggtcttcgtgctgctcgacgtgctgtgcttactggtcgcctccctgcccttcgctatcctgacgctggtgaacgccccgtacaagcgaggattttactgcggggatgactccatccggtacccctaccgtccagataccatcacccacgggctcatggctggggtcaccatcacggccaccgtcatccttgtctcggccggggaagcctacctggtgtacacagaccggctctattctcgctcggacttcaacaactacgtggctgctgtatacaaggtgctggggaccttcctgtttggggctgccgtgagccagtctctgacagacctggccaagtacatgattgggcgtctgaggcccaacttcctagccgtctgcgaccccgactggagccgggtcaactgctcggtctatgtgcagctggagaaggtgtgcaggggaaaccctgctgatgtcaccgaggccaggttgtctttctactcgggacactcttcctttgggatgtactgcatggtgttcttggcgctgtatgtgcaggcacgactctgttggaagtgggcacggctgctgcgacccacagtccagttcttcctggtggcctttgccctctacgtgggctacacccgcgtgtctgattacaaacaccactggagcgatgtccttgttggcctcctgcagggggcactggtggctgccctcactgtctgctacatctcagacttcttcaaagcccgacccccacagcactgtctgaaggaggaggagctggaacggaagcccagcctgtcactgacgttgaccctgggcgaggctgaccacaaccactatggatacccgcactcctcctcctgaggccggaccccgcccaggcagggagctgctgtgagtccagctgatgcccacccaggtggtccctccagcctggttaggcactgagggttctggacgggctccaggaaccctgggctgatgggagcagtgagcggttccgctgccccctgccctgcactggaccaggagtctggagatgcctgggtagccctcagcatttggaggggaacctgttcccgtcggtccccaaatatccccttctttttatggggttaaggaagggaccgagagatcagatagttgctgttttgtaaaatgtaatgtatatgtggtttttagtaaaatagggcacctgtttcacaaaaaaaaaaaaaaaaaaa

SEQ ID NO: 11 - DNA, 894 bp, Homo sapiens
taaggagcgagtcgaggtcgagctctatttaggtgacactatagaaccagacgtgactccgcggccgggccgggacgcgacgggacgcgctgggaccggcgtcgggggtcgcggggaccatgcagcggaggtgggtcttcgtgctgctcgacgtgctgtgcttactggtcgcctccctgcccttcgctatcctgacgctggtgaacgccccgtacaagcgaggattttactgcggggatgactccatccggtacccctaccgtccagataccatcacccacgggctcatggctggggtcaccatcacggccaccgtcatccttgtctcggccggggaagcctacctggtgtacacagaccggctctattctcgctcggacttcaacaactacgtggctgctgtatacaaggttgtctttctactcgggacactcttcctttgggatgtactgcatggtgttcttggcgctgtatgtgcaggcacgactctgttggaagtgggcacggctgctgcgacccacagtccagttcttcctggtggcctttgccctctacgtgggctacacccgcgtgtctgattacaaacaccactggagcgatgtccttgttggcctcctgcagggggcactggtggctgccctcactgtctgctacatctcagacttcttcaaagcccgacccccacagcactgtctgaaggaggaggagctggaacggaagcccagcctgtcactgacgttgaccctgggcgaggctgaccacaaccactatggatacccgcactcctcctcctgaggccggaccccgcccaggcagggagctactgtgagtccagctgaggcccacccaggtggtccctccagccctggttaggcactgagggctctggacggg

SEQ ID NO: 12 - DNA, 1286 bp, Homo sapiens
ctcctctccgcgcggggcgggctccgcgccacgtgactccgcggccgggccgggacgcgacgggacgcgctgggaccggcgtcgggggtcgcggggaccatgcagcggagcctccctgcccttcgctatcctgacgctggtgaacgccccgtacaagcgaggattttactgcggggatgactccatccggtacccctaccgtccagataccatcacccacgggctcatggctggggtcaccatcacggccaccgtcatccttgtctcggccggggaagcctacctggtgtacacagaccggctctattctcgctcggacttcaacaactacgtggctgctgtatacaaggtgctggggaccttcctgtttggggctgccgtgagccagtctctgacagacctggccaagtacatgattgggcgtctgaggcccaacttcctagccgtctgcgaccccgactggagccgggtcaactgctcggtctatgtgcagctggagaaggtgtgcaggggaaaccctgctgatgtcaccgaggccaggttgtctttctactcgggacactcttcctttgggatgtactgcatggtgttcttggcgctgtatgtgcaggcacgactctgttggaagtgggcacggctgctgcgacccacagtccagttcttcctggtggcctttgccctctacgtgggctacacccgcgtgtctgattacaaacaccactggagcgatgtccttgttggcctcctgcagggggcactggtggctgccctcactgtctgctacatctcagacttcttcaaagcccgacccccacagcactgtctgaaggaggaggagctggaacggaagcccagcctgtcactgacgttgaccctgggcgaggctgaccacaaccactatggatacccgcactcctcctcctgaggccggaccccgcccaggcagggagctgctgtgagtccagctgaggcccacccaggtggtccctccagccctggttaggcactgagggctctggacgggctccaggaaccctgggctgatgggagcagtgagcgggctccgctgccccctgccctgcactggaccaggagtctggagatgcctgggtagccctcagcatttggaggggaacctgttcccgtcggtccccaaatatccccttctttttatggggttaaggaagggaccgagagatcagatagttgctgttttgtaaaatgtaatgtatatgtggtttttagtaaaatagggcacctgtttcacaaaaaaaaaaaaaaaaaaa

SEQ ID NO: 13 - DNA, 1394 bp, Homo sapiens
ccctggaggcggggacctcgcgtgaggaccagacctccctccgcgccccgcactgcgcgcgccctcccaggctcgggggtccccgcgtcccaggcccagggggatgggggtcgcgagaggcccggggagccggggccagcatcccccgccccggcagcaggaagtctgtgcggaggggccgcgcgcgcgcctccatcccgccccgcctggcctgggagcctccctgcccttcgctatcctgacgctggtgaacgccccgtacaagcgaggattttactgcggggatgactccatccggtacccctaccgtccagataccatcacccacgggctcatggctggggtcaccatcacggccaccgtcatccttgtctcggccggggaagcctacctggtgtacacagaccggctctattctcgctcggacttcaacaactacgtggctgctgtatacaaggtgctggggaccttcctgtttggggctgccgtgagccagtctctgacagacctggccaagtacatgattgggcgtctgaggcccaacttcctagccgtctgcgaccccgactggagccgggtcaactgctcggtctatgtgcagctggagaaggtgtgcaggggaaaccctgctgatgtcaccgaggccaggttgtctttctactcgggacactcttcctttgggatgtactgcatggtgttcttggcgctgtatgtgcaggcacgactctgttggaagtgggcacggctgctgcgacccacagtccagttcttcctggtggcctttgccctctacgtgggctacacccgcgtgtctgattacaaacaccactggagcgatgtccttgttggcctcctgcagggggcactggtggctgccctcactgtctgctacatctcagacttcttcaaagcccgacccccacagcactgtctgaaggaggaggagctggaacggaagcccagcctgtcactgacgttgaccctgggcgaggctgaccacaaccactatggatacccgcactcctcctcctgaggccggaccccgcccaggcagggagctgctgtgagtccagctgaggcccacccaggtggtccctccagccctggttaggcactgagggctctggacgggctccaggaaccctgggctgatgggagcagtgagcgggctccgctgccccctgccctgcactggaccaggagtctggagatgcctgggtagccctcagcatttggaggggaacctgttcccgtcggtccccaaatatccccttctttttatggggttaaggaagggaccgagagatcagatagttgctgttttgtaaaatgtaatgtatatgtggtttttagtaaaatagggcacctgtttcacaaaaaaaaaaaaaaaaaaa

(19) United States
(12) Patent Application Publication
(10) Pub. No.: US 2010/0015047 A1
(43) Pub. Date: Jan. 21, 2010

(54) Lung-Expressed Polypeptides

(75) Inventors: Kevin Hestir, Kensington, CA (US); Ernestine Lee, Kensington, CA (US); Keting Chu, Woodside, CA (US); Yan Wang, Redwood City, CA (US); Kirsten Pierce, Burlingame, CA (US); Amy L. Tsui Collins, Oakland, CA (US); Lewis Thomas Williams, Mill Valley, CA (US)

Correspondence Address: FivePrime/Finnegan, 901 New York Avenue NW, Washington, DC, 20001-4413, US

(73) Assignee: FIVE PRIME THERAPEUTICS, INC.

(21) Appl. No.: 12/551,449

(22) Filed: Aug. 31, 2009

Related U.S. Application Data
(62) Division of application No. 10/543,838, filed on Feb. 23, 2007.
(60) Provisional application No. 60/443,944, filed on Jan. 31, 2003.
(60) Provisional application No. 60/444,913, filed on Feb. 3, 2003.
(60) Provisional application No. 60/446,647, filed on Feb. 10, 2003.
(60) Provisional application No. 60/448,837, filed on Feb. 18, 2003.

(51) Int. Cl. A61K 51/00; A61K 39/395

(52) U.S. Cl. 424/1.49; 424/174.1; 424/178.1; 424/183.1

(57) ABSTRACT

Modulators of phosphatidic acid phosphatase type 2C and other polypeptides, highly expressed in cancers as compared to normal tissues, are provided for treatment of proliferative disorders such as cancer. A method is provided for detecting polypeptides that are overexpressed in cancer, whereby antibodies or binding proteins that specifically recognize these molecules are contacted with a patient's bodily fluid. The method provides an early diagnosis of cancer, and can detect recurrence and metastasis following an initial diagnosis. The invention further provides methods of treating cancer with therapeutic agents directed toward these protein and peptide biomarkers.

### PRIORITY CLAIM

This application is related to U.S. application 60/444,944, “Methods of Use of Human Lung-Expressed Polypeptides Encoded by Polynucleotides and Antibodies Thereto,” filed Jan. 31, 2003; U.S. application 60/444,913, “Methods of Use of Human Lung-Expressed Polypeptides Encoded by Polynucleotides and Antibodies Thereto,” filed Feb. 3, 2003; U.S. application 60/446,647, “Methods of Use of Human Lung-Expressed Polypeptides Encoded by Polynucleotides and Antibodies Thereto,” filed Feb. 10, 2003; and U.S. application 60/448,837, “Methods of Use of Human Lung-Expressed Polypeptides Encoded by Polynucleotides and Antibodies Thereto,” filed Feb. 18, 2003, the contents of all of which are incorporated herein by reference in their entirety.

### TECHNICAL FIELD

This invention relates to human polynucleotides, and their encoded polypeptides which are highly expressed in cancer tissues, such as lung cancer, including adenocarcinomas and squamous cell carcinomas, bladder cancer, ovarian cancer, breast cancer, stomach cancer, colon cancer, kidney cancer, and pancreatic cancer. The invention also relates to modulators of such polynucleotides and polypeptides, for example, antibodies, that specifically bind to or interfere with the activity of these polypeptides, polynucleotides, their fragments, variants, and antagonists. The invention further relates to compositions containing such polypeptides, polynucleotides, or modulators thereof and uses of such compositions in methods of treating immune and proliferative disorders, including cancer and psoriasis. The polypeptides herein include, for example, human phosphatidic acid phosphatase 2C (PPAP2C) protein, cornichon-like protein, integrin alpha chain, alpha 6 protein, chromosome 1 Clorf9 protein, claudin 3 protein homologous to Clostridium perfringens enterotoxin receptor 2, KIAA0911 protein, hepatocyte growth factor activator inhibitor type 2 protein, coated vesicle membrane protein, BET1 protein, phosphatidylethanolamine N-methyltransferase protein, and others, and variants thereof. The invention additionally relates to methods of diagnosing immune disorders and proliferative disorders, such as cancer, by detecting these polynucleotides, polypeptides or antibodies thereto in patient samples. The invention provides diagnostic tests which identify polypeptides and polynucleotides herein that correlate with particular disorders.

### BACKGROUND ART

The American Cancer Society estimates that approximately 1,300,000 new cases of cancer will be diagnosed in the United States in 2003, and that approximately 550,000 cancer patients will die of the disease. An estimated 170,000 of these new cases will be diagnosed as lung cancer, and an estimated 160,000 patients will die of lung cancer in 2003. Lung cancer is the leading cause of cancer death in both men and women, and carries an especially poor prognosis. While the 5 year survival rate for all cancers combined is 62%, the 5 year survival rate for lung cancer is only 15%. This is because most lung cancers are not detected until the disease has reached an advanced stage; tumor stage is the most significant determinant of survival. When lung cancer is detected at an early stage, the 5 year survival rate climbs to 49% (American Cancer Society, 2003). Therefore, diagnostic markers for early stage lung cancer will have a significant impact on the morbidity and mortality of this disease.

Detection of cancer cell-specific biomarkers provides an effective screening strategy. Their early detection provides not only early diagnosis, but also the ability to screen for and detect post-operative residual tumor cells, and for occult metastases, an early indicator of tumor recurrence. Early detection can thus improve survival in patients before diagnosis, while undergoing treatment, and while in remission.

It would be desirable to provide novel methods and compositions for the treatment of cancers, such as lung and other cancers, and other proliferative and inflammatory diseases that are more efficacious and have a better safety profile than the currently available treatment modalities. It would also be desirable to provide better diagnostic tests for such diseases.

### DISCLOSURE OF THE INVENTION

The inventors have discovered that the human polynucleotides and polypeptides described in the Tables and Sequence Listing herein, are useful as targets for production of therapeutic agents for treatment of diseases in mammals, such as humans. The therapeutic agents of the present invention include modulators that are either agonists, antagonists, or fragments of these targets. For example, the polypeptides described herein can be used as immunogens in the production of specific antibody modulators directed against such polypeptides or their ligands, where the antibodies can be agonist antibodies or antagonist antibodies.

The modulators include not only antibodies, but also small molecule drugs, RNAi molecules, ribozymes, anti-sense molecules, soluble receptors or extracellular fragments of receptors, or transmembrane proteins. The polypeptides and polynucleotides herein are characterized in that they are highly expressed in tumor tissues in comparison with the expression levels in normal tissue. These therapeutic agents can be used in treating diseases such as proliferative or immune-related diseases. Cancer and psoriasis are two examples of commonly known proliferative diseases. Inflammatory bowel disease, multiple sclerosis, and rheumatoid arthritis are three of the commonly known immune-related diseases. However, the therapeutic agents herein can be used for treatment of other diseases besides these.

The inventors discovered that the targets herein are useful in screening assays for screening for modulators as above that have the desired agonist or antagonist effect.

The inventors have discovered that the polypeptides herein are transmembrane proteins or fragments thereof that are particularly suitable as targets for production of modulators. For example, the antibody modulators herein can bind such polypeptides on cell surfaces, such as tumor cell surface, to induce an antibody dependent cell cytotoxicity (ADCC) response, a cell dependent cytotoxicity (CDC) response, or in targeting delivery of cytotoxic molecules. The small molecule modulators and the soluble receptors or extracellular fragments of transmembrane proteins can block ligand/receptor interaction and interfere with cell signaling. The RNAi molecules, anti-sense molecules, and ribozymes can block expression of the target polypeptides.

The inventors have also discovered that compositions containing such polypeptides, polynucleotides and modulators, such as antibody modulators, can be used in methods of treatment of diseases as above. In particular, the inventors have found that certain targets are particularly desirable for the production of modulators such as antibodies because of the low level of expression of such polypeptides in normal tissues, such as in normal lung, heart, kidney and liver.

The inventors have further discovered methods for treatment of the foregoing diseases using the foregoing compositions where such treatment includes administering an appropriate composition to a subject either systemically or locally. The inventors have also discovered methods for diagnosis of diseases using the foregoing polypeptides, polynucleotides, and modulators.

### DEFINITIONS

The term “disease” refers to any disease, condition, infection, disorder or syndrome that requires medical intervention or for which medical intervention is desirable. Such medical intervention includes treatment, diagnosis, or prevention.

“Cancer” is herein defined as any abnormal cell or tissue growth, e.g., a tumor, that can be malignant or non-malignant. It is characterized by uncontrolled proliferation of cells that may or may not invade the surrounding tissue and, hence, may or may not metastasize to new body sites. Cancer encompasses carcinomas, which are cancers of epithelial cells; carcinomas include squamous cell carcinoma, adenocarcinoma, melanomas, and hepatomas. Cancer also encompasses sarcomas, which are tumors of mesenchymal origin, and includes osteogenic sarcomas, leukemias, and lymphomas. Cancers can involve one or more neoplastic cell type.

The term “overexpressed” or “highly expressed” refers to a state wherein there exists any measurable increase in expression over normal or baseline levels. For example, a molecule that is overexpressed in a disease is one that is manifest in a measurably higher level in the presence of the disease than in the absence of the disease. Such an increase can be at least two-fold at least three-fold, or more.

The term “binds specifically,” in the context of antibody binding, refers to high avidity and/or high affinity binding of an antibody to a specific polypeptide or a portion of the polypeptide, that is, an epitope of a polypeptide. Antibody binding to a specific epitope can be stronger than binding of the same antibody to any other epitopes, particularly other epitopes that can be present in molecules in association with, or in the same sample as the polypeptide of interest. For example, when an antibody binds more strongly to one epitope than to another, adjusting the binding conditions can result in antibody binding almost exclusively to the specific epitope and not to any other epitopes on the same polypeptide, and not to any other polypeptide which does not comprise the epitope. Antibodies that bind specifically to a subject polypeptide may be capable of binding other polypeptides at a weak, yet detectable, level (e.g., 10% or less of the binding shown to the polypeptide of interest). In general, antibodies of the invention bind to a specific polypeptide with a binding affinity of 10⁻⁷ M or greater (e.g., 10⁻⁸ M, 10⁻⁹ M, 10⁻¹⁰, 10⁻¹¹, etc.).

The term “host cell” includes an individual cell or cell culture which can be or has been a recipient of any recombinant vector(s) or isolated polynucleotide. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention may be called a “recombinant host cell.”

“Biological sample,” as used herein, includes biological fluids such as blood, serum, plasma, urine, cerebrospinal fluid, tears, saliva, lymph, dialysis fluid, lavage fluid, semen, and other liquid samples or tissues of biological origin. It includes cells or cells derived therefrom and the progeny thereof, including cells in culture, cell supernatants, and cell lysates. It includes organ or tissue culture-derived fluids, tissue biopsy samples, tumor biopsy samples, stool samples, and fluids extracted from physiological tissues. Cells dissociated from solid tissues, tissue sections, and cell lysates are included. The definition also includes samples that have been manipulated in any way after their procurement, such as by treatment with reagents, solubilization, or enrichment for certain components, such as polynucleotides or polypeptides. Also included in the term are derivatives and fractions of biological samples. A biological sample can be used in a diagnostic or monitoring assay.

The terms “subject,” “individual,” “host,” and “patient,” used interchangeably herein, refer to mammals, including, but not limited to, rodents, simians, humans, felines, canines, equines, bovines, porcines, ovines, caprines, mammalian laboratory animals, mammalian farm animals, mammalian sport animals, and mammalian pets.

The term “polypeptide” refers to a sequence of at least three, or at least four, or at least five, or at least six contiguous amino acid residues. Thus, “polypeptides” include full length proteins that include a signal peptide or leader sequence, if present, or a mature protein after cleavage of the signal peptide or leader sequence, the signal peptide or leader sequence, or portions of the full length or mature protein. “Polypeptides” include analogues and variants thereof. such as polymorphic variants. An active portion or fragment of a polypeptide is one that has activity such as the ability to act as an epitope for generation of antibodies, or one that contains a Pfam or enzymatic domain, or is sufficient to participate in a signal transduction pathway, or can be attached, for example.

An “epitope” is a sequence of amino acid residues in a polypeptide that may or may not be contiguous, and constitutes the antigen to which an antibody will bind.

The term “polynucleotide,” a “nucleic acid molecule,” or a “nucleotide sequence” refers to a polymer of nucleotides that encodes a polypeptide herein.

An “isolated,” “purified,” “substantially isolated,” or “substantially purified” antibody is one that has been manipulated to exist in a higher concentration than in nature. For example, a subject antibody is isolated, purified substantially isolated, or substantially purified when at least 10%, or 20%, or 40%, or 50%, or 70%, or 90% of non-subject-antibody materials with which it is associated in nature have been removed. As used herein, an “isolated,” “purified,” “substantially isolated,” or “substantially purified” polypeptide includes recombinant antibodies.

An “antibody” herein refers to an immunoglobulin molecule or an active fragment of such, including for example, a Fab fragment, a variable or constant region of a heavy chain, a variable or constant region of a light chain, a complementarity determining region (cdr), or a framework region. Thus, the antibody can be a monoclonal antibody, a polyclonal antibody, or a single chain antibody. The antibody can also be a neutralizing antibody, an agonist, or an antagonist. The antibody can be a fusion molecule linked to a cytotoxic molecule. The antibody can comprise a TCR or other backbone.

A “humanized” antibody is an antibody that contains mostly human immunoglobulin sequences. This term is generally used to refer to a non-human immunoglobulin that has been modified to incorporate portions of human sequences. A humanized antibody may include a human antibody that contains entirely human immunoglobulin sequences.

“Antibody-dependent cell cytotoxicity” (ADCC) is a form of lymphocyte mediated cytotoxicity in which an effector cell, such as a lymphocyte, mediates the killing of a cell to which an antibody is attached. Cell dependent cytotoxicity (CDC) is an adverse effect on a cell that results from an action of the cellular immune system.

A “signal peptide,” or a “leader sequence,” comprises a sequence of amino acid residues, typically, at the N terminus of a polypeptide, which directs the intracellular trafficking of the polypeptide. Polypeptides that contain a signal peptide or leader sequence typically also contain a signal peptide or leader sequence cleavage site. Such polypeptides, after cleavage at the cleavage sites, generate mature polypeptides after extracellular secretion or after being directed to the appropriate intracellular compartment.

A “biologically active” or “active” entity is one having structural, regulatory, or biochemical functions of a naturally occurring molecule. Biologically active fragments are those exhibiting activity similar, but not necessarily identical, to an activity of a nucleic acid, or polypeptide, or antibody of the present invention. The biological activity of the fragments can include an improved desired activity, or a decreased undesirable activity. For example, a biologically active fragment of a polynucleotide includes one that can be detected as unique for the polynucleotide molecule, or that can be used as a primer in PCR; and a biologically active fragment of a polypeptide includes one that can participate in a biological reaction, for example, in ligand/receptor interaction, in eliciting an immune response, such as production of antibodies, or that can participate in signal transduction, such as by binding to receptors, and/or activating enzymes or substrates.

The term “agonist” refers to a substance that mimics the function of an active molecule. Agonists include, but are not limited to, antibodies, growth factors, cytokines, lymphokines, small molecule drugs, hormones, and neurotransmitters, as well as analogues and fragments thereof.

The term “antagonist” refers to a molecule that interferes with the activity or binding of an agonist such as by competing for the binding sites of an agonist, but does not induce an active response.

The term “receptor” refers to a polypeptide that binds to a specific ligand, which is usually an extracellular molecule and upon binding, usually initiates a cellular response.

The term “ligand” refers to any molecule that binds to a specific site on another molecule, usually a receptor.

The term “modulate” encompasses an increase or a decrease, a stimulation, inhibition, interference, or blockage in a measured activity when compared to a suitable control.

A “modulator” of the polypeptides or polynucleotides or an “agent” herein is a molecule that interferes with the binding or activity of such polypeptides or polynucleotides. Such modulators or agents include, for example, polypeptide variants, whether agonist or antagonist; antibodies, whether agonist or antagonist; soluble receptors, usually antagonists; small molecule drugs, whether agonist or antagonist; RNAi, usually an antagonist; antisense molecules, usually an antagonist; and ribozymes, usually an antagonist. In some embodiments, an agent is a subject polypeptide, where the subject polypeptide itself is administered to an individual. In some embodiments, an agent is an antibody specific for a subject “target” polypeptide. In some embodiments, an agent is a chemical compound such as a small molecule that may be useful as an orally available drug. Such modulation includes the recruitment of other molecules that directly effect the modulation. For example, an antibody that modulates the activity of a subject polypeptide that is a receptor on a cell surface may bind to the receptor and fix complement, activating the complement cascade and resulting in lysis of the cell. An agent which modulates a biological activity of a subject polypeptide or polynucleotide increases or decreases the activity or binding at least about 10%, at least about 15%, at least about 20%, at least about 25%, at least about 50%, at least about 100%, or at least about 2-fold, at least about 5-fold, or at least about 10-fold or more when compared to a suitable control.

“Modulating a level of active subject polypeptide” includes increasing or decreasing activity of a subject polypeptide, increasing or decreasing a level of active polypeptide protein, and increasing or decreasing a level of mRNA encoding active subject polypeptide.

“Treatment,” as used herein, covers any treatment of a condition or disease in a mammal, including a human, and includes preventing the condition or disease from occurring or recurring in a subject who may be predisposed to the condition or disease but has not yet been diagnosed as having it, inhibiting the condition or disease, i.e., arresting its development, or relieving the condition or disease, i.e., causing regression of the condition or disease, or restoring or repairing a lost, missing, or defective function, or stimulating an inefficient process.

A “pharmaceutically acceptable carrier” refers to a non-toxic solid, semisolid or liquid filler, diluent, encapsulating material or formulation auxiliary of any conventional type. A pharmaceutically acceptable carrier is non-toxic to recipients at the dosages and concentrations employed and is compatible with other ingredients of the formulation. For example, the carrier for a formulation containing polypeptides does not include oxidizing agents and other compounds that are known to be deleterious to polypeptides. Suitable carriers include, but are not limited to, water, dextrose, glycerol, saline, ethanol, and combinations thereof. The carrier can contain additional agents such as wetting or emulsifying agents, pH buffering agents, or adjuvants which enhance the effectiveness of the formulation. Topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents can be added as necessary. Percutaneous penetration enhancers such as Azone can also be included.

The term “antibody target” refers to a polypeptide or a polynucleotide that can be used as an immunogen in the production of antibodies that specifically bind to such polypeptide or polynucleotide.

A “composition” of modulators, polypeptides, or polynucleotides herein refers to a composition that usually contains a pharmaceutically acceptable carrier or excipient that is conventional in the art and which is suitable for administration into a subject for therapeutic, diagnostic, or prophylactic purposes. For example, compositions for oral administration can form solutions, suspensions, tablets, pills, capsules, sustained release formulations, oral rinses, or powders.

It is to be understood that both the foregoing general description and the following detailed description are exemplary and explanatory only and are not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claims.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

### Target Molecules

Phosphatidic Acid Phosphatase Type 2C (PAP2C or PPAP2C)

Phosphatidic acid phosphatases (PPAP) convert phosphatidic acid to diacylglycerol in the biosynthetic pathway of structural membrane lipids, contributing to the de novo synthesis of glycerolipids. Phosphatidic acid and glycerolipids, such as diacylglycerol, are mediators of lipid signal transduction, in particular, transduction mediated by phospholipase D. By regulating these biosynthetic pathways, PPAP are involved in regulating lipid-mediated signal transduction.

The human phosphatidic acid phosphatase type 2C (PAP2C) gene is present on human chromosome 19, and localized to 19p13. It comprises 1327 base pairs, and encodes a gene product of 288 amino acids, with a predicted molecular mass of 32,577 daltons (Roberts et al., 1998). PAP2C is 54% identical to PAP2A and 43% identical to PAP2; all three encode integral membrane gene products with six transmembrane regions, a single consensus N-glycosylation site at amino acid residue 140, and a catalytic site for membrane-associated PAP activity. The catalytic sites are located in the second and third extracellular loops. Kanoh et al. (1999) suggest that the type 2 PAPs may act as ecto-enzymes to dephosphorylate exogenous substrates. The C-terminal amino acids of PAP2A, PAP2B, and PAP2C are widely divergent. Three alternatively spliced transcript variants encoding distinct isoforms have been reported for the PAP2C gene (Roberts et al., 1998).

The inventors have discovered that PAP2C (sometimes also referred to as PPAP2C) is highly expressed in human tumors such as malignant bladder, liver, ovary, breast, colon, kidney, pancreas, and lung, including adenocarcinomas and squamous cell carcinomas. The inventors have further discovered that this gene is expressed at low or very low levels in normal human lung, pancreas, and liver, and is almost undetectable in normal human adrenals, heart, kidney, and bladder. Thus, an antibody directed at PAP2C for therapeutic purposes is desirable as it is less likely to cause toxicity in important normal tissues and organs.

Collagen Type XI Alpha 1 (Col11A1)

The COL11A1 gene is present on human chromosome 1, and is comprised of 6319 base pairs, which encode an 1806 amino acid gene product. The primary transcripts of COL11A1 undergo differential splicing, resulting in at least six different variants (Zhidkova et al., 1995). The sequence of COL11A1 is disclosed through the NCBI as NM_{—}001854.

The COL11A1 gene encodes an N-terminal signal peptide, followed by a propeptide sequence that folds the collagen chain into its characteristic triple helical configuration with other chains, before the heterotrimer is cleaved to produce mature Type XI collagen. The COL11A1 propeptide sequence is different in length and structure than the propeptide sequences of many other procollagen alpha chains (Yoshioka and Ramirez, 1990). The COL11A1 propeptide comprises a globular domain, a collagenous region, and a nonhelical segment, which connects the propeptide domain to the next segment, which comprises the mature, cleaved, helical type XI collagen alpha 1 chain. This short helical segment has a defined cleavage site that separates fully processed type XI collagen from its propeptide (Yoshioka and Ramirez, 1990).

The inventors have discovered that COL11A is highly expressed in human malignant pancreas, lung, colon, ovary, liver, bladder, and breast, as compared to their normal counterparts. Moreover, this gene is not expressed or only expressed at low levels in normal human adrenals, heart, kidney, liver, and bladder. Thus, an antibody directed against COL11A is desirable as a therapeutic agent as it is less likely to cause toxicity in important normal tissues and organs.

Integrin α-11 Subunit (ITGA11)

The ITGA11 gene is present on human chromosome 15, and located at 15q22.3-q23. It is comprised of 3983 nucleotides, which encode an 1188 amino acid gene product. The ITGA11 gene comprises a signal peptide and a mature protein (Velling et al., 1999). Most of the ITGA11 protein resides extracellularly. Amino acids 1-1141 are extracellular, amino acids 1142-1164 span the membrane, and amino acids 1165-1188 reside within the cell cytoplasm. Amino acids 804-826 diverge from other integrin alpha chain sequences, and distinguish ITGA11 from other integrin alpha chains (Velling et al., 1999).

The inventors have discovered that although ITGA11 is highly expressed in human lung adenocarcinomas, lung squamous cell carcinomas, and colon adenocarcinomas, it is also highly expressed in human heart tissues, and is expressed in lung and kidney tissues. An antibody directed against ITGA11 may cause undesirable toxicity against heart tissues and to a lesser extent against lung and kidney as well.

Migration Inhibitory Factor (MIF)

Migration Inhibitory Factor (MIF) is a proinflanirnmatory chemotactic cytokine that is secreted from macrophages, T-cells, the pituitary gland, and several types of solid cancers. MIF is also produced by the endothelial cells of several organs, including the skin, eye, brain, kidney, and the lung. In the embryonic chicken lens, MIF expression is correlated with cellular differentiation (Tomiyasu et al., 2002). MIF is involved in cell cycle regulation; it induces degradation of the cyclin-dependent kinase inhibitor p27^{kip-1}.

The inventors have found that MIF is highly expressed in human lung adenocarcinomas, lung squamous cell carcinomas, and in colon adenocarcinomas. However, MIF is also highly expressed in normal human heart and kidney and expressed to a lesser extent in lung and kidney, rendering it less desirable as a target for therapeutic antibody intervention because of potential toxicity to important normal tissues or organs.

Human Hyaluronan Binding Protein (HABP2)

The HABP2 gene, also known as the plasma hyaluronan binding protein (PHBP) gene, is present on human chromosome 10, and localized to 10q25-q26 (Sumiya et al., 1997). It is comprised of 2408 base pairs. The gene is expressed in liver, kidney, and pancreas (Choi-Miura et al., 1996). The sequence of HABP2 is disclosed through the NCBI as S83182.

The inventors have found that HABP2 is highly expressed in human lung adenocarcinomas. However, this gene is also highly expressed in normal human kidney and liver, rendering this gene undesirable as a target for therapeutic antibody intervention because of possible toxicity to the kidney and liver.

Carboxypeptidase D Precursor (CPD)

Human carboxypeptidase D (CPD) is a membrane bound metallocarboxypeptidase that is optimally active at an acidic pH. The gene is comprised of 8025 base pairs, and has an open reading frame of 4131 base pairs encoding 1377 amino acid residues (Tan et al., 1997).

The predicted gene product has a signal peptide and a transmembrane anchor near the C-terminus. Between these there are three tandem carboxypeptidase homology domains with sequence similarity to the regulatory B-type carboxypeptidase family. The three repeats render carboxypeptidase D about three times larger (175-180 kDa) than other members of its family (approx. 50-62 kDa).

The inventors have found that CPD is highly expressed in human lung adenocarcinomas, lung squamous cell carcinomas, colon adenocarcinomas, and malignant pancreas. This gene is also somewhat highly expressed in normal human lung, and to a lesser extent in normal human heart, kidney and liver.

Protein Tyrosine Phosphatase Receptor Type F (PTPRF)

Protein tyrosine phosphatase receptor type F (PTPRF) is also referred to as the leukocyte antigen-related (LAR) tyrosine phosphatase. Protein tyrosine phosphatases are regulatory signaling molecules that mediate a variety of cellular processes including cell growth, differentiation, the mitotic cycle, and oncogenic transformation. Disruption in phosphatase regulated pathways of cell growth and programmed cell death can lead to abnormal cell growth, such as that which occurs in cancer.

The inventors have found that PTPRF is expressed in a number of normal human tissues including adrenals, kidney, liver, lung, breast, colon, prostate, and pancreas and highly expressed in malignant ovary, lung adenocarcinomas, lung squamous cell carcinomas, and colon adenocarcinomas.

Chromosome 1 Open Reading Frame 9; Membrane Protein CH1 (Chr1 Orf9)

The Chr1 Orf9 gene comprises 5556 base pairs, and encodes an open reading frame of 1254 amino acids (Rosok et al., 2000). It is located on human chromosome 1, at region 1q24, spans approximately 78.7 kb and is organized into at least 24 exons (Rosok et al., 2000). The sequence of Chr1 Orf9 is disclosed through the NCBI as NM_{—}014283.

The inventors have found that Chr1 Orf9 is expressed in normal human adrenals, heart, kidney, liver, lung, pancreas. This gene is overexpressed in malignant human bladder, liver, ovary, breast, pancreas, and colon adenocarcinomas.

Plexin A3

The plexin A3, or SEX, gene, is a likely human ortholog of the mouse plexin 3 gene, which was derived from a mouse brain cDNA library, and comprises 6039 base pairs. It is the human analogue of mouse plexin 3, a receptor that associates with a tyrosine kinase activity via its cytoplasmic domain, and triggers a signal transduction pathway controlling cell repulsion among epithelial cells (Tamagnone et al., 1999; Kameyama et al., 1996).

The inventors have found that the plexin A3 gene is highly expressed in human lung adenocarcinomas, lung squamous cell carcinomas, and colon adenocarcinomas. However, this gene is also expressed in normal human lung, heart, and kidney and, to a lesser extent, in liver. When compared to normal human counterparts, this gene is overexpressed in malignant bladder, liver, ovary, stomach, breast, colon, lung, prostate, and kidney.

KIAA0466

A partial coding sequence comprising 6588 base pairs of an mRNA was derived from a size-fractionated human brain cDNA library. This putative KIAA0466 gene is located on chromosome 1, and is predicted to encode a 1214 amino acid gene product (Seki et al., 1997).

The inventors have found that KIAA0466 is highly expressed in human lung squamous cell carcinomas. This gene is also found to be expressed in lung adenocarcinomas, colon adenocarcinomas, normal lung, heart, kidney, and, to a much lesser extent, liver.

Beta-1,4-Galactosyltransferase I (B4GALT)

The B4GALT gene is present on chromosome 1, and is localized to 1p33-p34. It is comprised of 1888 base pairs, and is predicted to encode an amino acid gene product of 373 amino acids (Lo et al., 1998). Beta1,4-galactosyltransferases are localized in the trans-Golgi compartment of most eukaryotic cells, where they participate in the elongation of oligosaccharide chains on glycoproteins and glycolipids.

The inventors have found that this gene is highly expressed in human lung adenocarcinomas, lung squamous cell carcinomas, and colon adenocarcinomas. It is also expressed in normal human lung, heart, kidney and liver. In paired comparisons, this gene is overexpressed in malignant bladder, liver, ovary, stomach, breast, and lung.

Panel

These tumor markers can be used in combination, e.g., in a panel that comprises two or more markers. It is expected that almost all lung cancers will overexpress at least one of these genes, and that combining these markers into a panel will provide a comprehensive screen for certain cancers.

### Gene Expression of the Target Molecules in Cancer

The present invention utilized probes and primers that were either purchased directly from Applied Biosystems, Inc. (ABI) (Foster City, Calif.) Assay-On-Demand, or were designed using software PrimerExpress. The exact probe and primer sequences that were purchased from ABI were not released. However, the approximate amplicon sequences could be estimated based on the information provided from ABI.

As an example, to order PPAP2C, it can be searched under Assay ID Hs00186575 from the website: http://myscience.applied biosystems.com/cdsEntry/Form/gene_expression_keyword.jsp. Under “Interrogated Sequence,” on the webpage, it is shown that the amplicon covers exon boundaries of exon 3 and exon 4.

The “assay location” nucleotide 579 was shown to be within the amplicon sequence when using RefSeq sequence number, NM_{—}003712. In addition, the “context sequence” provided by ABI (TGTCACCGAGGCCAGGTTGTCTTTC for PPAP2C) was shown to be a sequence within the amplicon. The map view link also provided some information about the amplicon. Taken together, the amplicon was about 75-150 bp in length and covered the “assay location” nucleotide, the “context sequence,” as well as the exon 3 and 4 boundary.

The level of gene expression was examined in individual normal and cancer tissue samples. Some normal samples were taken from regions adjacent to cancer tissue. The relative gene expression level in cancer and normal tissue was analyzed based on the threshold cycle in quantitative real-time PCR. The expression of each sample (cancer or normal tissue) was normalized to its own internal control 18S rRNA expression and represented by ½^{ΔCt}. ΔCt for cancer tissue equals to 2^{Ct(gene}^{_{—}}^{C)-Ct(18S}^{_{—}}^{C)} and ΔCt for normal tissue equals 2^{Ct(gene}^{_{—}}^{n)-Ct(18S}^{_{—}}ⁿ⁾ for normal tissue.

The present inventors also interrogated a proprietary oncology database from GeneLogic, using Affymetrix U133 chip probe IDs that corresponded to certain of the sequences studied herein to determine the expression of the sequences in normal tissues and in cancer tissues.

Gene Expression of PAP2C

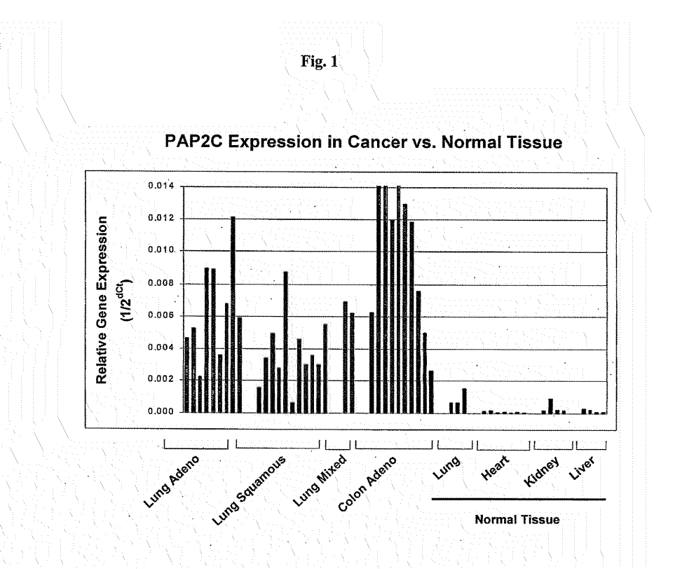

As shown in FIG. 1, PAP2C was found to be highly expressed in at least 8 out of 9 human lung adenocarcinomas, 9 out of 11 human lung squamous cell carcinomas, and 10 out of 10 human colon adenocarcinomas of cancer patients (“Cancer”), as compared to an average expression level in normal tissues of normal individuals (“Normal Tissue”). The expression of the PAP2C gene in normal lung, heart, kidney, and liver tissues was found to be low or very low.

Further, interrogation of the GeneLogic database showed overexpression of this gene in malignant bladder, liver, ovary, breast, colon, lung, kidney and pancreas as compared to expression in the corresponding normal tissues. PAP2C, thus, is a strong target for production of therapeutic antibodies for treatment of tumors in which this gene is over or highly expressed because of the low probability of causing toxic side effects to the important normal tissues and organs.

Gene Expression of COL11A1

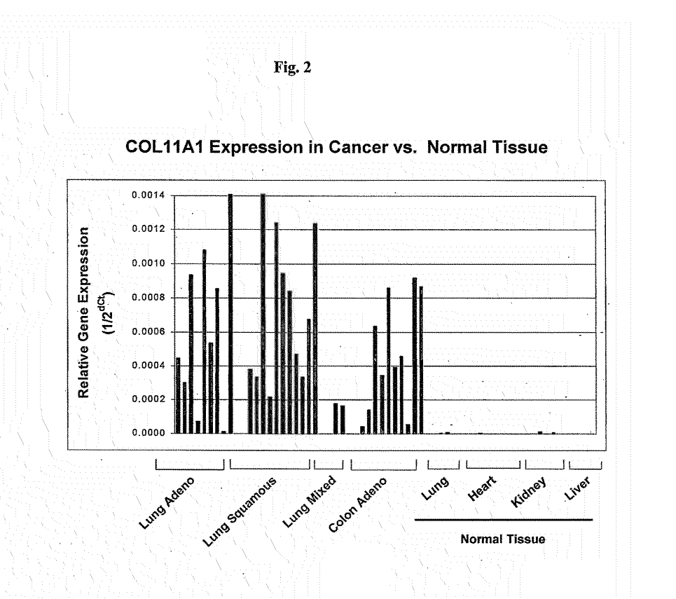

As shown in FIG. 2, COL11A1 was over or highly expressed in 7 out of 9 human lung adenocarcinomas, 10 out of 11 human lung squamous cell carcinomas, and 7 out of 10 human colon adenocarcinomas of cancer patients (“Cancer”) as compared with Normal Tissue. In contrast, this gene was barely detectable in normal human lung, heart, kidney, or liver.

Interrogation of the GeneLogic database showed overexpression of Col11A1 in malignant bladder, liver, ovary, stomach, breast, colon, lung, and pancreas compared to its level of expression in the corresponding normal tissues. The COL11A1 gene was found to be either not expressed, or was expressed at a low level in a small percent of normal adrenals, heart, kidney, liver, lung, bladder, prostate, and pancreas.

COL11A 1, thus, is a strong target for production of therapeutic antibodies for treatment of tumors in which this gene is over or highly expressed because of the low probability of causing toxic side effects to the important normal tissues and organs. This gene is also useful as a tumor biomarker gene for diagnostic testing purposes in the serum and/or tissues of humans.

Gene Expression of ITGA11

ITGA11 gene was found to be highly expressed in 6 out of 9 human lung adenocarcinomas, about 4 out of 11 human lung squamous cell carcinomas, and about 7 out of 10 human colon adenocarcinomas of cancer patients. However, this gene was also found to be expressed at a high level, though not as high level as in the tumor tissues, in 3 out of 3 normal human lung samples, 6 out of 7 normal human heart samples, and 3 out of 4 normal human kidney samples.

Gene Expression of HABP2

The HABP2 gene was found to be highly expressed in 4 out of 9 human lung adenocarcinomas, about 1 out of 11 human lung squamous cell carcinomas, and about 2 out of 10 human colon adenocarcinomas of cancer patients. However, this gene was also found to be highly expressed in 4 out of 4 normal human kidney and 4 out of 4 normal liver samples.

Gene Expression of MIF

The MIF gene was found to be highly expressed in about 6 out of 9 human lung adenocarcinomas, about 10 out of 11 human lung squamous cell carcinomas, and about 7 out of 10 human colon adenocarcinomas of cancer patients. However, this gene was also found to be expressed at a high level in about 3 out of 7 normal human heart samples, and 3 out of 4 normal human kidney samples and at a lower but significant level in 3 out of 3 normal human lung samples and 4 out of 4 liver samples.

Gene Expression of CPD

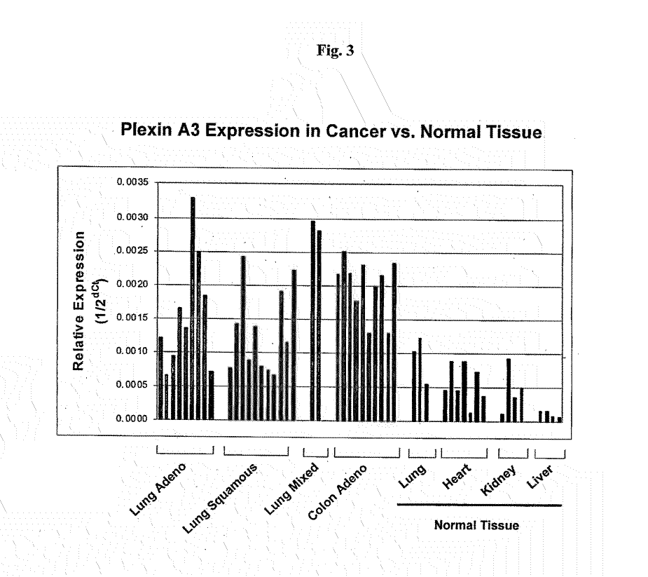

As shown in FIG. 3, the CPD gene was found to be highly expressed in 9 out of 9 human lung adenocarcinomas, 11 out of 11 human lung squamous cell carcinomas, and about 8 out of 10 human colon adenocarcinomas (“Cancer”) of cancer patients. However, this gene was also found to be expressed at a high level in 2 out of 3 normal human lung samples, 4 out of 7 normal human heart samples, and 3 out of 4 normal human kidney samples and 4 out of 4 normal human liver samples.

Gene Expression of PTPRF (LAR)

The PTPRF or LAR gene was found to be highly expressed in 5 out of 9 human lung adenocarcinomas, about 10 out of 11 human lung squamous cell carcinomas, and 8 out of 10 human colon adenocarcinomas of cancer patients. This gene was also found to be expressed at a high level or a significant level in 3 out of 3 normal human lung samples, 4 out of 4 normal human kidney samples, and 4 out of 4 normal human liver samples.

Gene Expression of Chr1 Orf9

The Chr1 Orf9 gene was found to be highly expressed in 2 out of 9 human lung adenocarcinomas, about 4 out of 11 human lung squamous cell carcinomas, and about 6 out of 10 human colon adenocarcinomas of cancer patients. This gene was also found to be expressed at a high level in 1 out of 3 normal human lung samples and 3 out of 7 normal human heart samples. This gene is also expressed at a significant level in 1 out of 3 normal human lung samples, 1 out of 7 normal human heart samples, and 2 out of 4 normal human kidney samples.

Gene Expression of Plexin A3

The Plexin A3 gene was found to be highly expressed in 9 out of 9 human lung adenocarcinomas, 11 out of 11 human lung squamous cell carcinomas, and 10 out of 10 human colon adenocarcinomas of cancer patients. However, this gene was also found to be highly expressed or expressed at a significant level in 3 out of 3 normal human lung samples, about 6 out of 7 normal human heart samples, and 3 out of 4 normal human kidney samples.

Gene Expression of KIAA0466

The KIAA0466 gene was found to be highly expressed in 3 out of 9 human lung adenocarcinomas, about 8 out of 11 human lung squamous cell carcinomas, and about 2 out of 10 human colon adenocarcinomas of cancer patients. This gene was also found to be expressed at a high or significant level in 1 out of 3 normal human lung samples, about 4 out of 7 normal human heart samples, and 4 out of 4 normal human kidney samples.

Gene Expression of Beta-1,4-Galactosyltransferase I

The beta 1,4-galactosyltransferase I gene was found to be highly expressed in 7 out of 9 human lung adenocarcinomas, 10 out of 11 human lung squamous cell carcinomas, and 9 out of 10 human colon adenocarcinomas of cancer patients. This gene was also found to be expressed at a high or significant level in 2 out of 3 normal human lung samples, 6 out of 7 normal human heart samples, 4 out of 4 normal human kidney samples, and 4 out of 4 normal human liver samples.

### Cancer Cell Markers in Body Fluids

Genes that are uniquely or differentially expressed in cancerous cells or tissues may potentially serve as cancer cell markers in bodily fluids, e.g., serum. A reliable marker must be specific to cancer, and expressed only when the patient has cancer. Recently, the ceruloplasmin gene was identified to be overexpressed in cancer, and reported to be elevated in patient serum. Serum ceruloplasmin is increased over normal in lung cancer patients before treatment, falls during treatment, and rises again upon tumor recurrence. However, ceruloplasmin is an unsuitable serum biomarker because it is an acute phase reactive protein that is elevated in many non-specific physiological responses. It is elevated in non-malignant lung disease, in smokers, and in various malignant and non-malignant diseases (Wang et al., 2002).

### Protein Families

The polypeptides herein comprise PAP2 protein family domains (“Pfam”). The “Pfam” system is an organization of protein sequence classification and analysis, based on conserved protein domains; it can be publicly accessed in a number of ways, for example, at http://pfam.wustl.edu. Protein domains are portions of proteins that have a tertiary structure and sometimes have enzymatic or binding activities; multiple domains can be connected by flexible polypeptide regions within a protein. Pfam domains can comprise the N-terminus or the C-terminus of a protein, or can be situated at any point in between. The Pfam system identifies protein families based on these domains and provides an annotated, searchable database that classifies proteins into families.

Sequences encompassed by the invention include, but are not limited to, the polypeptide and polynucleotide sequences of the molecules shown in the tables, figures and Sequence Listing herein, as well as corresponding molecular sequences found at all developmental stages of an organism. Sequences of the invention can comprise genes or gene segments designated in the application, and their gene products, i.e., RNA and polypeptides. They also include variants of those presented in the tables, figures and Sequence Listing herein that are present in the normal physiological state, e.g., variant alleles such as SNPs, and splice variants, as well as variants that are affected in pathological states, such as disease-related mutations or sequences with alterations that lead to pathology, and variants with conservative amino acid changes.

Some of the sequences disclosed in the tables, figures and Sequence Listing herein comprise one or more PAP2 superfamily (PAP2) domains. This family includes the enzyme type 2 phosphatidic acid phosphatase (PAP2), glucose-6-phosphatase EC:3.1.3.9, Phosphatidylglycerophosphatase B EC:3.1.3.27, and bacterial acid phosphatase EC:3.1.3.2, as well as other phosphoesterases. This domain is present in a number of proteins, including bacitracin transport permease and glucose 6-phosphatase. The structure of this domain is known (http://pfam.wustl.edu/cgi-bin/getdesc?name=PAP2).

### Active Agents (or Modulators)

The nucleic acid, polypeptide, and modulator compositions of the subject invention find use as therapeutic agents in situations where one wishes to modulate an activity of a subject polypeptide in a host, particularly the activity of the subject polypeptides, or to provide or inhibit the activity at a particular anatomical site. Thus, the compositions are useful in treating disorders associated with an activity of a subject polypeptide. The following provides further details of active agents of the present invention.

Antisense Oligonucleotides

In certain embodiments of the invention, the active agent is an agent that modulates, and generally decreases or down regulates, the expression of a gene encoding a target protein in a host, i.e., antisense molecules. Anti-sense reagents include antisense oligonucleotides (ODN), i.e., synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g., by reducing the amount of mRNA available for translation, through activation of RNase H, or steric hindrance. One or a combination of antisense molecules can be administered, where a combination can comprise multiple different sequences.

Antisense molecules can be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides can be chemically synthesized by methods known in the art (Wagner et al., 1993; Milligan et al., 1993) Oligonucleotides can be chemically modified from the native phosphodiester structure to increase their intracellular stability and binding affinity, for example, as described in detail above. Antisense oligonucleotides will generally be at least about 7, at least about 12, or at least about 20 nucleotides in length, and not more than about 500, not more than about 50, or not more than about nucleotides in length, where the length is governed by efficiency of inhibition, and specificity, including absence of cross-reactivity, and the like. Short oligonucleotides, of from about 7 to about 8 bases in length, can be strong and selective inhibitors of gene expression (Wagner et al., 1996).

A specific region or regions of the endogenous sense strand mRNA sequence is chosen to be complemented by the antisense sequence. Selection of a specific sequence for the oligonucleotide can use an empirical method, where several candidate sequences are assayed for inhibition of expression of the target gene in an in vitro or animal model. A combination of sequences can also be used, where several regions of the mRNA sequence are selected for antisense complementation.

As an alternative to anti-sense inhibitors, catalytic nucleic acid compounds, e.g., ribozymes, or anti-sense conjugates can be used to inhibit gene expression. Ribozymes can be synthesized in vitro and administered to the patient, or can be encoded in an expression vector, from which the ribozyme is synthesized in the targeted cell (WO 9523225; Beigelman et al., 1995). Examples of oligonucleotides with catalytic activity are described in WO 9506764. Conjugates of anti-sense ODN with a metal complex, e.g., terpyridyl Cu(II), capable of mediating mRNA hydrolysis are described in Bashkin et al., 1995.

Interfering RNA

In some embodiments, the active agent is an interfering RNA (RNAi), including dsRNAi. RNA interference provides a method of silencing eukaryotic genes. Double stranded RNA can induce the homology-dependent degradation of its cognate mRNA in C. elegans, fungi, plants, Drosophila, and mammals (Gaudilliere et al., 2002). Use of RNAi to reduce a level of a particular mRNA and/or protein is based on the interfering properties of double-stranded RNA derived from the coding regions of a gene. The technique reduces the time between identifying an interesting gene sequence and understanding its function, and thus is an efficient high-throughput method for disrupting gene function (O'Neil, 2001). RNAi can also help identify the biochemical mode of action of a drug and to identify other genes encoding products that can respond or interact with specific compounds.

In one embodiment of the invention, complementary sense and antisense RNAs derived from a substantial portion of the subject polynucleotide are synthesized in vitro. The resulting sense and antisense RNAs are annealed in an injection buffer, and the double-stranded RNA injected or otherwise introduced into the subject, i.e., in food or by immersion in buffer containing the RNA (Gaudilliere et al., 2002; O'Neil et al., 2001; WO99/32619). In another embodiment, dsRNA derived from a gene of the present invention is generated in vivo by simultaneously expressing both sense and antisense RNA from appropriately positioned promoters operably linked to coding sequences in both sense and antisense orientations.

### Peptides and Modified Peptides

In some embodiments of the present invention, the active agent is a peptide. Suitable peptides include peptides of from about 3 amino acids to about 50, from about 5 to about 30, or from about 10 to about 25 amino acids in length. In some embodiments, a peptide has a sequence of from about 3 amino acids to about 50, from about 5 to about 30, or from about 10 to about 25 amino acids of corresponding naturally-occurring protein. In some embodiments, a peptide exhibits one or more of the following activities: inhibits binding of a subject polypeptide to an interacting protein or other molecule; inhibits subject polypeptide binding to a second polypeptide molecule; inhibits a signal transduction activity of a subject polypeptide; inhibits an enzymatic activity of a subject polypeptide; or inhibits a DNA binding activity of a subject polypeptide.

Peptides can include naturally-occurring and non-naturally occurring amino acids. Peptides can comprise D-amino acids, a combination of D- and L-amino acids, and various “designer” amino acids (e.g., β-methyl amino acids, Cα-methyl amino acids, and Nα-methyl amino acids, etc.) to convey special properties. Additionally, peptides can be cyclic. Peptides can include non-classical amino acids in order to introduce particular conformational motifs. Any known non-classical amino acid can be used. Non-classical amino acids include, but are not limited to, 1,2,3,4-tetrahydroisoquinoline-3-carboxylate; (2S,3 S)-methylphenylalanine, (2S,3R)-methyl-phenylalanine, (2R,3S)-methyl-phenylalanine and (2R,3R)-methyl-phenylalanine; 2-aminotetrahydronaphthalene-2-carboxylic acid; hydroxy-1,2,3,4-tetrahydroisoquinoline-3-carboxylate; β-carboline (D and L); HIC (histidine isoquinoline carboxylic acid); and HIC (histidine cyclic urea). Amino acid analogs and peptidomimetics can be incorporated into a peptide to induce or favor specific secondary structures, including, but not limited to, LL-Acp (LL-3-amino-2-propenidone-6-carboxylic acid), a β-turn inducing dipeptide analog; β-sheet inducing analogs; β-turn inducing analogs; α-helix inducing analogs; γ-turn inducing analogs; Gly-Ala turn analogs; amide bond isostere; or tetrazol, and the like.

A peptide can be a depsipeptide, which can be linear or cyclic (Kuisle et al., 1999). Linear depsipeptides can comprise rings formed through S—S bridges, or through an hydroxy or a mercapto group of an hydroxy-, or mercapto-amino acid and the carboxyl group of another amino- or hydroxy-acid but do not comprise rings formed only through peptide or ester links derived from hydroxy carboxylic acids. Cyclic depsipeptides contain at least one ring formed only through peptide or ester links, derived from hydroxy carboxylic acids.

Peptides can be cyclic or bicyclic. For example, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Methods for making cyclic peptides are well known in the art.

A desamino or descarboxy residue can be incorporated at the terminal ends of the peptide, so that there is no terminal amino or carboxyl group, to decrease susceptibility to proteases or to restrict conformation. C-terminal functional groups include amide, amide lower alkyl amide di (lower alkyl), lower alkoxy, hydroxy, and carboxy, and the lower ester derivatives thereof, and the pharmaceutically acceptable salts thereof.

In addition to the foregoing N-terminal and C-terminal modifications, a peptide or peptidomimetic can be modified with or covalently coupled to one or more of a variety of hydrophilic polymers to increase solubility and circulation half-life of the peptide. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran, and dextran derivatives. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky (1995); Monfardini et al. (1995); U.S. Pat. Nos. 4,640,835; 4,496,689; 4,301,144; 4,670,417; 4,791,192; 4,179,337, or WO 95/34326.

### Peptide Aptamers

Another suitable agent for modulating an activity of a subject polypeptide is a peptide aptamer. Peptide aptamers are peptides or small polypeptides that act as dominant inhibitors of protein function. Peptide aptamers specifically bind to target proteins, blocking their functional ability (Kolonin and Finley, 1998). Due to the highly selective nature of peptide aptamers, they can be used not only to target a specific protein, but also to target specific functions of a given protein (e.g., a signaling function). Further, peptide aptamers can be expressed in a controlled fashion by use of promoters which regulate expression in a temporal, spatial or inducible manner. Peptide aptamers act dominantly, therefore, they can be used to analyze proteins for which loss-of-function mutants are not available.

Peptide aptamers that bind with high affinity and specificity to a target protein can be isolated by a variety of techniques known in the art. Peptide aptamers can be isolated from random peptide libraries by yeast two-hybrid screens (Xu et al., 1997). They can also be isolated from phage libraries (Hoogenboom et al., 1998) or chemically generated peptides/libraries.

Soluble Receptors

Extracellular fragments of cell surface receptors can be soluble, aid can modulate a target protein. These fragments can act as ligands for binding to receptors on cell surfaces in ligand/receptor interactions, and modulate the receptors and cellular activity downstream of the receptors. This modulation can trigger certain intracellular responses, such as inducing signal transduction to activate cells or inhibit cellular activity, to induce cellular growth, proliferation, or differentiation, or to induce the production of other factors that, in turn, mediate such activities.

Small Molecules

Small molecule modulators such as those commonly used as therapeutic drugs can be used as inhibitors, agonists, antagonists, and the like. Small molecule agents include chemical compounds that bind the polypeptide and modulate activity of the polypeptide or cell containing the polypeptide. Small molecule modulators may permeate the cell, and/or may exert their action at the extracellular surface or on non-cellular structures, such as the extracellular matrix.

Antibodies

An antibody of the present invention may comprise a monoclonal antibody, polyclonal antibody, single chain antibody, intrabody, and active fragments of any of these. The active fragments include variable regions from either heavy chains or light chains. The antibody can comprise the backbone of a molecule with an immunoglobulin domain, e.g., a fibronectin backbone, a T-cell receptor (TCR) backbone, or a CTLA4 backbone.

The present invention further features a targeting antibody, a neutralizing antibody, a stabilizing antibody, an enhancing antibody, an antibody agonist, an antibody antagonist, an antibody that promotes cellular endocytosis of a target antigen, a cytotoxic antibody, and an antibody that mediates, complement-dependent cytotoxicity (CDC) or antibody dependent cellular cytotoxicity (ADCC). The antibody that mediates ADCC can deliver a payload, such as a cytotoxic component, e.g., a radioisotope, a radioactive molecule, a microbial toxin, a plant toxin, a chemotherapeutic agent, or a chemical substance, such as doxorubicin or cisplatin. The payload can be attached using technology from Seattle Genetics (Bothell, Wash.), which incorporates synthetic stable linkers and drugs that can be used to increase the potency of an antibody. These linkers are stable in the bloodstream but release drug payloads under conditions inside target cells.

The invention also features an inhibitory antibody, functioning to specifically inhibit the binding of a cognate polypeptide to its ligand or its substrate, or to specifically inhibit the binding of a cognate peptide as the substrate of another molecule.

The antibodies of the present invention also encompass a human antibody, a non-human primate antibody, e.g., monkey; a non-primate animal antibody, e.g., a rodent such as a rat, mouse, hamster, or guinea pig; a chicken antibody, a cattle antibody, such as a sheep, pig, cow, horse, or goat; a cat; a dog; and a rabbit. It also features a humanized antibody, a primatized antibody, and a chimeric antibody.

The antibodies and antibody fragments of the invention can be produced in vitro or in vivo. For example, the present invention features an antibody produced in a cell-free expression system, a prokaryote expression system or a eukaryote expression system, as described herein. For example, antibody fragments can be made in E. coli.

The invention further provides a host cell that can produce an antibody of the invention or a fragment thereof. The antibody may also be secreted by the cell. The host cell can be a hybridoma, or a prokaryotic or eukaryotic cell. The invention also provides a bacteriophage or other virus particle comprising an antibody of the invention, or a fragment thereof. The bacteriophage or other virus particle may display the antibody or fragment thereof on its surface, and the bacteriophage itself may exist within a bacterial cell. The antibody may also comprise a fusion protein with a viral or bacteriophage protein.

The invention further provides transgenic multicellular organisms, e.g., plants or non-human animals, as well as tissues or organs, comprising a polynucleotide sequence encoding a subject antibody or fragment thereof. The organism, tissues, or organs will generally comprise cells producing an antibody of the invention, or a fragment thereof.

In another aspect, the present invention features a method of making an antibody by immunizing a host animal (Coligan, 2002). In this method, a polypeptide or a fragment thereof, a polynucleotide encoding a polypeptide, or a polynucleotide encoding a fragment thereof, is introduced into an animal in a sufficient amount to elicit the generation of antibodies specific to the polypeptide or fragment thereof, and the resulting antibodies are recovered from the animal. The polypeptide or polynucleotide sequence can be chosen from the Sequence Listing or the Tables. Initial immunizations can be with either polynucleotide or polypeptide sequences. Subsequent booster immunizations can be with either polynucleotide or polypeptide sequences. Initial immunization with a polynucleotide can be followed with either polynucleotide or polypeptide immunizations, and an initial immunization with a polypeptide can be followed with either polynucleotide or polypeptide immunizations.

The invention provides antibodies that specifically recognize a particular polypeptide. Antibodies are obtained by immunizing a host animal with peptides, polynucleotides encoding polypeptides, or cells, each comprising all or a portion of the target protein. The host animal will generally be a different species than the immunogen, e.g., a human protein used to immunize mice. Methods of antibody production are well known in the art (Coligan, 2002; Howard and Bethell, 2000; Harlow et al., 1998; Harlow and Lane, 1988).

The invention thus also provides a non-human animal comprising an antibody of the invention. The animal can be a non-human primate, (e.g., a monkey) a rodent (e.g., a rat, a mouse, a hamster, a guinea pig), a chicken, cattle (e.g. a sheep, a goat, a horse, a pig, a cow), a rabbit, a cat, or a dog. Suitable host animals include rodents (e.g., mouse, rat, guinea pig, hamster), cattle (e.g., sheep, pig, cow, horse, goat), cat, dog, chicken, primate, monkey, and rabbit.

The present invention also features a method of making an antibody by isolating a spleen from an animal injected with a polypeptide or a fragment thereof, a polynucleotide encoding a polypeptide, or a polynucleotide encoding a fragment thereof, and recovering antibodies from the spleen cells. Hybridomas can be made from the spleen cells, and hybridomas secreting specific antibodies can be selected.

The present invention further features a method of making a polynucleotide library from spleen cells, and selecting a cDNA clone that produces specific antibodies, or fragments thereof. The cDNA clone or a fragment thereof can be expressed in an expression system that allows production of the antibody or a fragment thereof, as provided herein.

The immunogen can comprise a nucleic acid, a complete protein, or fragments and derivatives thereof, or proteins expressed on cell surfaces. Pfam domains can be used as immunogens. Transmembrane domains can also be used as immunogens. Additionally, non-transmembrane domains, e.g., extracellular, cytoplasmic, or luminal domains can be used as immunogens. Immunogens comprise all or a part of one of the subject proteins, where these amino acids contain post-translational modifications, such as glycosylation, found on the native target protein. Immunogens comprising protein extracellular domains are produced in a variety of ways known in the art, e.g., expression of cloned genes using conventional recombinant methods, or isolation from tumor cell culture supernatants, etc. The immunogen can also be expressed in vivo from a polynucleotide encoding the immunogenic peptide introduced into the host animal.

Polyclonal antibodies are prepared by conventional techniques. These include immunizing the host animal in vivo with the target protein (or immunogen) in substantially pure form, for example, comprising less than about 1% contaminant. The immunogen can comprise the complete target protein, fragments, or derivatives thereof. To increase the immune response of the host animal, the target protein can be combined with an adjuvant; suitable adjuvants include alum, dextran, sulfate, large polymeric anions, and oil & water emulsions, e.g., Freund's adjuvant (complete or incomplete). The target protein can also be conjugated to synthetic carrier proteins or synthetic antigens. The target protein is administered to the host, usually intradermally, with an initial dosage followed by one or more, usually at least two, additional booster dosages. Following immunization, blood from the host will be collected, followed by separation of the serum from blood cells. The immunoglobulin present in the resultant antiserum can be further fractionated using known methods, such as ammonium salt fractionation, or DEAE chromatography and the like. Cytokines can also be used to help stimulate immune response.

Cytokines act as chemical messengers, recruiting immune cells that help the killer T-cells to the site of attack. An example of a cytokine is granulocyte-macrophage colony-stimulating factor (GM-CSF), which stimulates the proliferation of antigen-presenting cells, thus boosting an organism's response to a cancer vaccine. As with adjuvants, cytokines can be used in conjunction with the antibodies and vaccines disclosed herein. For example, they can be incorporated into the antigen-encoding plasmid or introduced via a separate plasmid, and in some embodiments, a viral vector can be engineered to display cytokines on its surface.

The method of producing polyclonal antibodies can be varied in some embodiments of the present invention. For example, instead of using a single substantially isolated polypeptide as an immunogen, one may inject a number of different immunogens into one animal for simultaneous production of a variety of antibodies. In addition to protein immunogens, the immunogens can be nucleic acids (e.g., in the form of plasmids or vectors) that encode the proteins, with facilitating agents, such as liposomes, microspheres, etc, or without such agents, such as “naked” DNA.

Antibodies can also be prepared using a library approach. Briefly, mRNA is extracted from the spleens of immunized animals to isolate antibody-encoding sequences. The extracted mRNA may be used to make cDNA libraries. Such a cDNA library may be normalized and subtracted in a manner conventional in the art, for example, to subtract out cDNA hybridizing to mRNA of non-immunized animals. The remaining cDNA may be used to create proteins and for selection of antibody molecules or fragments that specifically bind to the immunogen. The cDNA clones of interest, or fragments thereof, can be introduced into an in vitro expression system to produce the desired antibodies, as described herein.

In a further embodiment, polyclonal antibodies can be prepared using phage display libraries, conventional in the art. In this method, a collection of bacteriophages displaying antibody properties on their surfaces are made to contact subject polypeptides, or fragments thereof. Bacteriophages displaying antibody properties that specifically recognize the subject polypeptides are selected, amplified, for example, in E. coli, and harvested. Such a method typically produces single chain antibodies.

Phage display technology can be used to produce Fab antibody fragments, which can be then screened to select those with strong and/or specific binding to the protein targets. The screening can be performed using methods that are known to those of skill in the art, for example, ELISA, immunoblotting, immunohistochemistry, or immunoprecipitation. Fab fragments identified in this manner can be assembled with an Fc portion of an antibody molecule to form a complete immunoglobulin molecule.

Monoclonal antibodies are also produced by conventional techniques, such as fusing an antibody-producing plasma cell with an immortal cell to produce hybridomas. Suitable animals will be used, e.g., to raise antibodies against a mouse polypeptide of the invention, the host animal will generally be a hamster, guinea pig, goat, chicken, or rabbit, and the like. Generally, the spleen and/or lymph nodes of an immunized host animal provide the source of plasma cells, which are immortalized by fusion with myeloma cells to produce hybridoma cells. Culture supernatants from individual hybridomas are screened using standard techniques to identify clones producing antibodies with the desired specificity. The antibody can be purified from the hybridoma cell supernatants or from ascites fluid present in the host by conventional techniques, e.g., affinity chromatography using antigen, e.g., the subject protein, bound to an insoluble support, i.e., protein A sepharose, etc.

The antibody can be produced as a single chain, instead of the normal multimeric structure of the immunoglobulin molecule. Single chain antibodies have been previously described (i.e., Jost et al., 1994). DNA sequences encoding parts of the immunoglobulin, for example, the variable region of the heavy chain and the variable region of the light chain are ligated to a spacer, such as one encoding at least about four small neutral amino acids, i.e., glycine or serine. The protein encoded by this fusion allows the assembly of a functional variable region that retains the specificity and affinity of the original antibody.

The invention also provides intrabodies that are intracellularly expressed single-chain antibody molecules designed to specifically bind and inactivate target molecules inside cells. Intrabodies have been used in cell assays and in whole organisms (Chen et al., 1994; Hassanzadeh et al., 1998). Inducible expression vectors can be constructed with intrabodies that react specifically with a protein of the invention. These vectors can be introduced into host cells and model organisms.

The invention also provides “artificial” antibodies, e.g., antibodies and antibody fragments produced and selected in vitro. In some embodiments, these antibodies are displayed on the surface of a bacteriophage or other viral particle, as described above. In other embodiments, artificial antibodies are present as fusion proteins with a viral or bacteriophage structural protein, including, but not limited to, M13 gene III protein. Methods of producing such artificial antibodies are well known in the art (U.S. Pat. Nos. 5,516,637; 5,223,409; 5,658,727; 5,667,988; 5,498,538; 5,403,484; 5,571,698; and 5,625,033). The artificial antibodies, selected for example, on the basis of phage binding to selected antigens, can be fused to a Fc fragment of an immunoglobulin for use as a therapeutic, as described, for example, in U.S. Pat. No. 5,116,964 or WO 99/61630. Antibodies of the invention can be used to modulate biological activity of cells, either directly or indirectly. A subject antibody can modulate the activity of a target cell, with which it has primary interaction, or it can modulate the activity of other cells by exerting secondary effects, i.e., when the primary targets interact or communicate with other cells. The antibodies of the invention can be administered to mammals, and the present invention includes such administration, particularly for therapeutic and/or diagnostic purposes in humans.

Antibodies may be administered by injection systemically, such as by intravenous injection; or by injection or application to the relevant site. such as by direct injection into a tumor, or direct application to the site when the site is exposed in surgery; or by topical application, such as if the disorder is on the skin. for example.

For in vivo use, particularly for injection into humans, in some embodiments it is desirable to decrease the antigenicity of the antibody. An immune response of a recipient against the antibody may potentially decrease the period of time that the therapy is effective. Methods of humanizing antibodies are known in the art. The humanized antibody can be the product of an animal having transgenic human immunoglobulin genes, e.g., constant region genes (e.g., Grosveld and Kolias, 1992; Murphy and Carter, 1993; Pinkert, 1994; and International Patent Applications WO 90/10077 and WO 90/04036). Alternatively, the antibody of interest can be engineered by recombinant DNA techniques to substitute the CH1, CH2, CH3, hinge domains, and/or the framework domain with the corresponding human sequence (see, e.g., WO 92/02190). Humanized antibodies can also be produced by immunizing mice that make human antibodies, such as Abgenix xenomice, Medarex's mice, or Kirin's mice, and can be made using the technology of Protein Design Labs, Inc. (Fremont, Calif.) (Coligan, 2002). Both polyclonal and monoclonal antibodies made in non-human animals may be humanized before administration to human subjects. The antibodies can be partially human or fully human antibodies. For example, xenogenic antibodies, which are produced in animals that are transgenic for human antibody genes, can be employed to make a fully human antibody. By xenogenic human antibodies is meant antibodies that are fully human antibodies, with the exception that they are produced in a non-human host that has been genetically engineered to express human antibodies (e.g., WO 98/50433; WO 98/24893 and WO 99/53049).

Chimeric immunoglobulin genes constructed with immunoglobulin cDNA are known in the art (Liu et al. 1987a; Liu et al. 1987b). Messenger RNA is isolated from a hybridoma or other cell producing the antibody and used to produce cDNA. The cDNA of interest can be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes are known in the art (Kabat et al., 1991). Human C region genes are readily available from known clones. The choice of isotype will be guided by the desired effector functions, such as complement fixation, or antibody-dependent cellular cytotoxicity. IgG1, IgG3 and IgG4 isotypes, and either of the kappa or lambda human light chain constant regions can be used. The chimeric, humanized antibody is then expressed by conventional methods.

Consensus sequences of heavy (“H”) and light (“L”) J regions can be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

A convenient expression vector for producing antibodies is one that encodes a functionally complete human CH or CL immunoglobulin sequence, with appropriate restriction sites engineered so that any VH or VL sequence can be easily inserted and expressed, such as plasmids, retroviruses, YACs, or EBV derived episomes, and the like. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody can be joined to any strong promoter, including retroviral LTRs, e.g., SV-40 early promoter, (Okayama, et al. 1983), Rous sarcoma virus LTR (Gorman et al. 1982), and Moloney murine leukemia virus LTR (Grosschedl et al. 1985), or native immunoglobulin promoters.

Antibody fragments, such as Fv, F(ab′)2, and Fab can be prepared by cleavage of the intact protein, e.g., by protease or chemical cleavage. These fragments can include heavy and light chain variable regions. Alternatively, a truncated gene can be designed, e.g., a chimeric gene encoding a portion of the F(ab′)₂ fragment that includes DNA sequences encoding the CH1 domain and hinge region of the H chain, followed by a translational stop codon. The antibodies of the present invention may be administered alone or in combination with other molecules for use as a therapeutic, for example, by linking the antibody to cytotoxic agent, as discussed above, or to a radioactive molecule. Radioactive antibodies that are specific to a cancer cell, disease cell, or virus-infected cell may be able to deliver a sufficient dose of radioactivity to kill such cancer cell, disease cell, or virus-infected cell. The antibodies of the present invention can also be used in assays for detection of the subject polypeptides. In some embodiments, the assay is a binding assay that detects binding of a polypeptide with an antibody specific for the polypeptide; the subject polypeptide or antibody can be immobilized, while the subject polypeptide and/or antibody can be detectably-labeled. For example, the antibody can be directly labeled or detected with a labeled secondary antibody. That is, suitable, detectable labels for antibodies include direct labels, which label the antibody to the protein of interest, and indirect labels, which label an antibody that recognizes the antibody to the protein of interest.

These labels include radioisotopes, including, but not limited to ⁶⁴Cu, ⁶⁷Cu, ⁹⁰Y, ¹²⁴I, ¹²⁵I, ¹³¹I, ¹³⁷Cs, ¹⁸⁶Re, ²¹¹At, ²¹²Bi, ²¹³Bi, ²²³Ra, ²⁴¹Am, and ²⁴⁴Cm; enzymes having detectable products (e.g., luciferase, P-galactosidase, and the like); fluorescers and fluorescent labels, e.g., as provided herein; fluorescence emitting metals, e.g., ¹⁵²Eu, or others of the lanthanide series, attached to the antibody through metal chelating groups such as EDTA; chemiluminescent compounds, e.g., luminol, isoluminol, or acridinium salts; and bioluminescent compounds, e.g., luciferin, or acquorin (green fluorescent protein), specific binding molecules, e.g., magnetic particles, microspheres, nanospheres, and the like.

Alternatively, specific-binding pairs may be used, involving, e.g., a second stage antibody or reagent that is detectably-labeled and that can amplify the signal. For example, a primary antibody can be conjugated to biotin, and horseradish peroxidase-conjugated strepavidin added as a second stage reagent. Digoxin and antidigoxin provide another such pair. In other embodiments, the secondary antibody can be conjugated to an enzyme such as peroxidase in combination with a substrate that undergoes a color change in the presence of the peroxidase. The absence or presence of antibody binding can be determined by various methods, including flow cytometry of dissociated cells, microscopy, radiography, or scintillation counting. Such reagents and their methods of use are well known in the art.

All of the immunogenic methods of the invention can be used alone or in combination with other conventional or unconventional therapies. For example, immunogenic molecules can be combined with other molecules that have a variety of antiproliferative effects, or with additional substances that help stimulate the immune response, i.e., adjuvants or cytokines.

### BRIEF DESCRIPTION OF THE TABLES AND DRAWINGS

### Tables

Table 1 lists the sequences in the Sequence Listing. Each is identified by a Five Prime Identification (FP ID) number, a SEQ ID NO. corresponding to the nucleotide coding sequence (SEQ ID NO. (N1)), a SEQ ID NO. corresponding to the encoded polypeptide sequence (SEQ ID NO. (P1)), and a SEQ ID NO. corresponding to the entire nucleotide sequence (SEQ ID NO. (N0)). Each is also identified by its public National Center for Information Biotechnology (NCBI) protein identification number (Protein ID).

Table 2 provides an annotated list of the sequences of the invention. Each sequence is identified by its FP ID and its NCBI protein identification number (Protein ID). An annotation is provided for each protein sequence, listing information about the protein and listing reference numbers through which more information about the protein can be obtained through the NCBI.

Table 3 provides information characteristic of each polypeptide. The polypeptides are identified by their FP ID. Each is classified according to its function, e.g., HG1014563 is a single transmembrane type 1 membrane protein (Classification). The length of the polypeptide is provided as the number of amino acid residues (Predicted Protein Length). Table 3 also specifies the result of an algorithm that predicts whether a sequence is secreted (Tree Vote). This algorithm is constructed on the basis of a number of attributes that include hydrophobicity, two-dimensional structure, prediction of signal sequence cleavage site, and other parameters. This algorithm predicts whether the sequences listed in Table 3 are secreted as indicated in the classification column; a higher Tree Vote indicates that the polypeptide is more likely to be secreted. The signal peptide coordinates (Signal Peptide Coords) are listed in terms of the amino acid residues beginning with 1 at the N-terminus of the polypeptide. The Mature Protein Coords refer to the coordinates of the amino acid residues of the mature polypeptide after cleavage of the signal peptide. Table 3 also specifies the coordinates of an alternative form of the mature protein (Alternate Mature Protein Coords). In instances where the mature protein start residue overlaps the signal peptide end residue, some of the amino acid residues may be cleaved off such that the mature protein does not start at the next amino acid residue from the signal peptides, resulting in the alternative mature protein coordinates. Finally, Table 3 provides the coordinates of the transmembrane and non-transmembrane sequences of the polypeptides. The transmembrane coordinates (TM Coords) refer to the transmembrane and are listed in terms of the amino acid residues beginning with 11 at the N-terminus of the polypeptide. The non-transmembrane coordinates (non-TM Coords) refer to the amino acids that are not transmembrane; these can include extracellular, cytoplasmic, and luminal sequences, and are listed in terms of the amino acid residues beginning with “1” at the N-terminus of the polypeptide.

Table 4 lists the coordinates of the Pfam domains of the polypeptides of the invention. Each is identified by a Five Prime Identification (FP ID)) number, and the public NCBI protein identification number (Protein ID). The Pfam domains of those polypeptides that have at least one Pfam domain are listed (Pfam) and the Pfam coordinates are listed in terms of amino residues beginning with “1” at the N-terminus of the polypeptide, beginning at the beginning of the open reading frame.

### DRAWINGS

FIG. 1: PAP2C Expression in Cancer vs. Normal Tissue. FIG. 1 shows the relative gene expression of PAP2C in lung adenocarcinoma (Lung adeno), lung squamous cell carcinoma (Lung squamous), mixed lung adenocarcinoma and squamous cell cancer (Lung mixed), and colon adenocarcinoma (Colon adeno). It also shows the relative gene expression of PAP2C in normal lung, heart, kidney, and liver.

FIG. 2: COL11A1 Expression in Cancer vs. Normal Tissue. FIG. 2 shows the relative gene expression of COL11A1 in lung adenocarcinoma (Lung adeno), lung squamous cell carcinoma (Lung squamous), mixed lung adenocarcinoma and squamous cell cancer (Lung mixed), and colon adenocarcinoma (Colon adeno). It also shows the relative gene expression of COL11A1 in normal lung, heart, kidney, and liver.

FIG. 3: Plexin A3 Expression in Cancer vs. Normal Tissue. FIG. 3 shows the relative gene expression of Plexin A3 in lung adenocarcinoma (Lung adeno), lung squamous cell carcinoma (Lung squamous), mixed lung adenocarcinoma and squamous cell cancer (Lung mixed), and colon adenocarcinoma (Colon adeno). It also shows the relative gene expression of Plexin A3 in normal lung, heart, kidney, and liver.

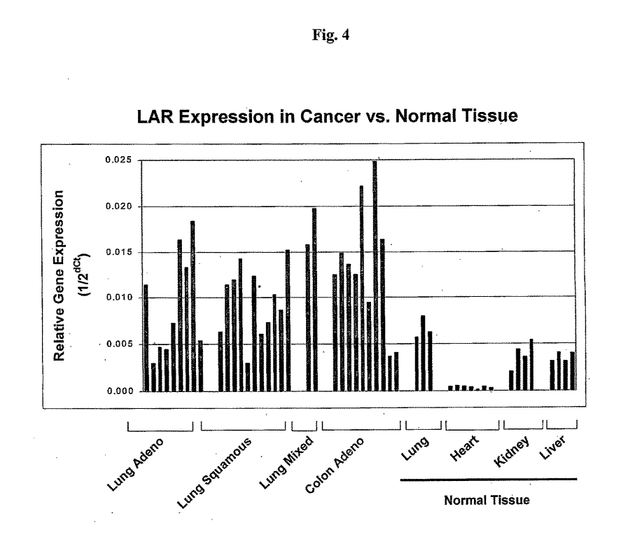

FIG. 4: LAR Expression in Cancer vs. Normal Tissue. FIG. 4 shows the relative gene expression of LAR in lung adenocarcinoma (Lung adeno), lung squamous cell carcinoma (Lung squamous), mixed lung adenocarcinoma and squamous cell cancer (Lung mixed), and colon adenocarcinoma (Colon adeno). It also shows the relative gene expression of LAR in normal lung, heart, kidney, and liver.

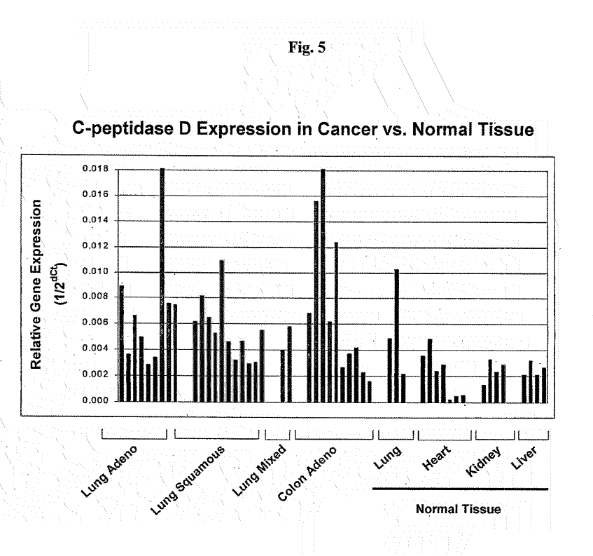

FIG. 5: C-peptidase D Expression in Cancer vs. Normal Tissue. FIG. 5 shows the relative gene expression of C-peptidase D in lung adenocarcinoma (Lung adeno), lung squamous cell carcinoma (Lung squamous), mixed lung adenocarcinoma and squamous cell cancer (Lung mixed), and colon adenocarcinoma (Colon adeno). It also shows the relative gene expression of C-peptidase D in normal lung, heart, kidney, and liver.

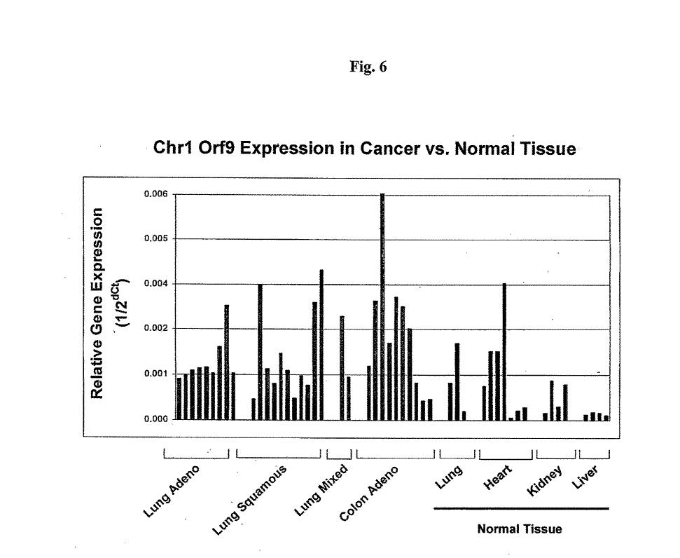

FIG. 6: Chr1 Orf9 Expression in Cancer vs. Normal Tissue. FIG. 6 shows the relative gene expression of Chr1 Orf9 in lung adenocarcinoma (Lung adeno), lung squamous cell carcinoma (Lung squamous), mixed lung adenocarcinoma and squamous cell cancer (Lung mixed), and colon adenocarcinoma (Colon adeno). It also shows the relative gene expression of Chr1 Orf9 in normal lung, heart, kidney, and liver.

### MODES FOR CARRYING OUT THE INVENTION

The invention provides polynucleotides and polypeptides, listed in the Sequence Listing and Tables. These polypeptides and polynucleotides have novel functions, and provide methods of diagnosis, treatment, and prophylaxis for immune disorders and cancer, including cancers of the lung, bladder, prostate, breast, liver, pancreas, kidney, ovary, cervix, skin, bone, brain, and gastrointestinal tract, such as esophagus, stomach, colon, and rectum, as well as soft tissue sarcomas, leukemias, and lymphomas. Some of these polypeptides comprise regions that correspond to pfam domains. The regions of the polypeptides that correspond to a particular pfam domain can exhibit variations among polypeptides. For example, fibroblast growth factor receptors of the invention comprise epidermal growth factor (EGF) domains, which have variable polypeptide sequences, and are encoded by variable nucleotide sequences.

The invention provides an isolated polynucleotide encoding a polypeptide or an isolated polypeptide encoded by the polynucleotide, wherein the polypeptide consists essentially of an amino acid sequence selected from among “non-TM Coords” in Table 3, “Pfam Coords” in Table 4, or the Sequence Listing. The amino acid sequence can be a sequence of at least 6 contiguous amino acid residues.

The invention also provides a method of making the polypeptides comprising providing a nucleic acid molecule that comprises a polynucleotide sequence that encodes the polypeptide, introducing the nucleic acid molecule into an expression system, and allowing expression of the polypeptide. The expression system can be a cell-free system, such as wheat germ extract, a rabbit reticulocyte, or a frog oocyte expression system. It can also be a bacterial expression system, a yeast expression system, an insect cell expression system, or a mammalian cell expression system.

The invention provides a pharmaceutical composition comprising a pharmaceutically acceptable carrier or excipient and the isolated polypeptide or isolated polynucleotide selected from the Tables, the “non-TM Coords” in Table 3, “Pfam Coords” in Table 4, or the Sequence Listing. The composition can comprise a phosphatidic acid phosphatase 2C polypeptide.

The invention also provides an isolated antibody specifically recognizing, binding to, and/or modulating the biological activity of at least one polypeptide or polynucleotide selected from the Tables, the “non-TM Coords” in Table 3, “Pfam Coords” in Table 4, or the Sequence Listing. The antibody can recognizing, bind to, and/or modulate the biological activity of phosphatidic acid phosphatase type 2 or variants thereof. The invention provides a pharmaceutical composition comprising a pharmaceutically acceptable carrier and such an antibody.

The antibody can be a monoclonal antibody, a polyclonal antibody, a single chain antibody, an antibody comprising a backbone of a molecule with an Ig domain or a TCR backbone, a targeting antibody, a neutralizing antibody, a stabilizing antibody, an enhancing antibody, an antibody agonist, an antibody antagonist, an antibody that promotes endocytosis of a target antigen, a cytotoxic antibody, an antibody that mediates ADCC, a human antibody, a non-human primate antibody, a non-primate animal antibody, a rabbit antibody, a mouse antibody, a rat antibody, a sheep antibody, a goat antibody, a horse antibody, a porcine antibody, a cow antibody, a chicken antibody, a humanized antibody, a primatized antibody, a chimeric antibody, an antigen binding fragment, a fragment comprising a variable region of a heavy chain or a light chain of an immunoglobulin, a fragment comprising a complementarity determining region or a framework region of an immunoglobulin, or other active fragments thereof, analogues thereof, and antagonists thereto. The antibody can comprise an antigen binding fragment of an immunoglobulin.

This antibody can be produced in a plant, an animal or in a cell. The cell can be a bacterial cell, a fungal cell, a plant cell, an insect cell, or a mammalian cell. The cell can also be a yeast cell, an Aspergillus cell, an SF9 cell, a High Five cell, a cereal plant cell, a tobacco cell, a tomato cell, or a CHO cell.

The antibody can comprise one or more cytotoxic component chosen from a radioisotope, a microbial toxin, a plant toxin, and a chemical compound. The antibody can function to specifically inhibit the binding of the polypeptide to a ligand, specifically inhibit the binding of the polypeptide to a substrate, specifically inhibit the binding of the polypeptide as a ligand, specifically inhibit the binding of the polypeptide as a substrate, induce apoptosis, or induce ADCC or CDC.

The antibody can recognize, bind to, and/or modulate the biological activity of collagen type11 alpha1, carboxypeptidase D precursor, F-receptor linked protein tyrosine phosphatase, chromosome 1 open reading frame 9, ortholog of mouse plexin 3, KIAA0466, or beta-1,4-galactosyltransferase.

The antibody can specifically bind to or interfere with the activity of a polypeptide or a ligand of the polypeptide. It can be directed to a polypeptide sequence of at least 6, at least 8, at least 10, at least 12, at least 14, at least 16, at least 18, at least 20, or at least 22 contiguous amino acid residues chosen from the Sequence Listing and/or Tables. These contiguous residues can correspond to one or more extracellular domain of a polypeptide, or fragment thereof, analogue thereof, and/or antagonist thereto. These residues can correspond to a pfam domain. The antibody may recognize one or more antigenic epitope. It may specifically recognize one variant of the pfam domain, or more than one variant.

In another aspect, the invention provides a method for making an antibody by introducing a polypeptide, polynucleotide encoding the polypeptide, or a biologically active fragment thereof, into an animal in sufficient amount to elicit generation of antibodies specific to the polypeptide, wherein the polypeptide is described in the Sequence Listing or Tables, and recovering the antibodies. This method may further entail isolating a spleen from the animal injected with the polypeptide or polynucleotide or a fragment thereof, and recovering the antibodies from the spleen cells. It may also further entail making a hybridoma using spleen cells and selecting a hybridoma that secretes the antibodies. The invention provides making a polynucleotide library from the spleen cells, selecting a cDNA clone that produces the antibodies, and expressing the cDNA clone in an expression system to produce antibodies or fragments thereof. The cDNA clone, or a fragment thereof, can be introduced into an expression system to produce the antibody. This expression system can be an in vitro system, such as a cell-free system, a bacterial cell expression system, a yeast expression system, or a mammalian sell expression system.

The antibody can be produced either in vivo or in vitro, and can be produced by either a prokaryote or a eukaryote, such as a bacterial cell, a fungal cell, a plant cell, an insect cell, and a mammalian cell. Examples of suitable cells include yeast cells, Aspergillus cells, SF9 cells, High Five cells, CHO cells, cereal plant cells, tobacco cells, and tomato cells. The antibody can be isolated.

The antibody can function to specifically inhibit the binding of the polypeptide to a ligand, specifically inhibit the binding of the polypeptide to a substrate, specifically inhibit the binding of the polypeptide as a ligand, and/or specifically inhibit the binding of the polypeptide as a substrate.

The invention provides a host cell that produces an antibody that can recognize, bind to, and/or modulate the biological activity of from the Tables, the “non-TM Coords” in Table 3, “Pfam Coords” in Table 4, or the Sequence Listing. It also provides a bacteriophage, wherein such an antibody, or a fragment thereof, is displayed on the bacteriophage. The antibody may be displayed on the surface of the bacteriophage. The invention also provides a bacterial cell comprising the bacteriophage. It further provides a host cell that secretes an antibody of the invention.

The invention also provides a non-human animal injected with the polypeptide or polynucleotide from the Tables, the “non-TM Coords” in Table 3, “Pfam Coords” in Table 4, or the Sequence Listing.

The invention further provides a method for determining the presence of a polypeptide specifically binding to an antibody in a sample by allowing the antibody as described above to interact with the sample; and determining whether interaction between the antibody and the polypeptide has occurred.

The invention provides a method for determining the presence of an antibody specifically binding to a polypeptide or a polynucleotide in a sample by allowing the polypeptide or polynucleotide from the Tables, the “non-TM Coords” in Table 3, “Pfam Coords” in Table 4, or the Sequence Listing. to interact with the sample; and determining whether interaction between the antibody and the polypeptide or polynucleotide has occurred.

The invention provides a method for modulating the biological activity of a first human or non-human animal host cell by providing an antibody as described above and contacting the antibody with a first host cell, wherein the activity of the first host cell, or a second host cell, is modulated. The modulation of biological activity can be chosen from enhancing cell activity directly, enhancing cell activity indirectly, inhibiting cell activity directly, inhibiting cell activity indirectly, inducing apoptosis, inducing ADCC, and inducing CDC. The cell activity that is modulated can be signal transduction, transcription, and/or translation. This modulation can result in cell death and/or inhibition of cell growth. Contacting the antibody with a first host cell can result in recruitment of at least one second host cell. The first host cell can be a cancer cell. The first or second host cell can be a T cell, B cell, NK cell, dendritic cell, macrophage, muscle cell, stem cell, skin cell, fat cell, blood cell, brain cell, bone marrow cell, endothelial cell, retinal cell, bone cell, kidney cell, pancreatic cell, liver cell, spleen cell, prostate cell, cervical cell, ovarian cell, breast cell, lung cell, soft tissue cell, colorectal cell, or a cell of the gastrointestinal tract.

In a further aspect, the invention provides a method for modulating biological activity by providing an antibody, such as one described above, and contacting this antibody with a first human or non-human host cell, thereby modulating the activity of a first human or non-human animal host cell, or a second host cell. Modulators also take the form of small molecule modulators. The modulation of biological activity can take the form of enhancing cell activity directly, enhancing cell activity indirectly, inhibiting cell activity directly, and/or inhibiting cell activity indirectly. It can also take the form of modulating signal transduction, transcription, and/or translation. Modulation can result in cell growth, inhibition of cell growth and/or cell death.

One way this modulation can occur is by contacting the antibody with a first human or non-human host cell to result in the recruitment of the second host cell. The first host cell can, for example, be a cancer cell. Either the first or second host cell can be a T cell, B cell, NK cell, dendritic cell, macrophage, muscle cell, stem cell, skin cell, fat cell, blood cell, brain cell, bone marrow cell, endothelial cell, retinal cell, bone cell, kidney cell, pancreatic cell, liver cell, spleen cell, prostate cell, cervical cell, ovarian cell, breast cell, lung cell, liver cell, soft tissue cell, colorectal cell, or gastrointestinal tract cell.

The invention provides a method for screening for a modulator of polypeptide activity by providing a composition comprising a polypeptide or an active fragment thereof, wherein the polypeptide is chosen from the Sequence Listing or Table 1, allowing at least one modulator to contact the polypeptide, and selecting a modulator that binds to the polypeptide or interferes with the activity of the polypeptide. The polypeptide can be expressed on a cell surface. It can be an antibody. A modulator selected in this manner can be present in a composition with a pharmaceutically acceptable carrier.

The invention provides a method for identifying a modulator that modulates the biological activity of a polypeptide comprising providing at least one polypeptide chosen from among Table 1, the Pfam Coords in Table 4, the non-TM Coords in Table 3, and active fragments thereof by allowing at least one agent to contact the polypeptide; and selecting an agent that binds the polypeptide or affects the biological activity of the polypeptide. The polypeptide can be phosphatidic acid phosphatase type 2C. The polypeptide can also be collagen type11 alpha1, carboxypeptidase D precursor, F-receptor linked protein tyrosine phosphatase, chromosome 1 open reading frame 9, ortholog of mouse plexin 3, KIAA0466, or beta-1,4-galactosyltransferase. The modulator can be an antibody, a small molecule drug, a soluble receptor, or an extracellular fragment of the polypeptide.

The invention provides a modulator composition comprising a modulator and a pharmaceutically acceptable carrier, wherein the modulator is chosen from among one obtainable by the methods and antibodies described above, a soluble receptor that competes for ligand binding to the polypeptide of claim 1 an extracellular fragment that competes for ligand binding to the polypeptide of claim 1, a RNAi molecule, an anti-sense molecule, or a ribozyme that inhibits the transcription or translation of the polynucleotide.

In yet a further aspect, the invention provides a method for diagnosing a proliferative disease such as cancer, psoriasis, and ulcerative colitis, or an immune or inflammatory disease such as rheumatoid arthritis, osteoarthritis, psoriasis, inflammatory bowel disease, and multiple sclerosis, by providing an antibody, allowing the antibody to contact a patient sample, and detecting specific binding between the antibody and an antigen in the sample to determine whether the subject has proliferative disease such as cancer. The invention also provides a method for diagnosing a proliferative disease, by providing a polypeptide that specifically binds the antibody, allowing the polypeptide to contact a patient sample, and detecting specific binding between the polypeptide and any interacting molecule in the sample to determine whether the subject has a proliferative disease.

The invention provides a method for diagnosing cancer in a patient by providing an antibody described above, and allowing it to contact a patient sample, and detecting specific binding between the antibody and an antigen in the sample to determine whether the subject has cancer.

The invention also provides a method for diagnosing cancer in a patient by providing a method for diagnosing cancer in a patient, by providing a polypeptide that specifically binds an antibody as described above, allowing the polypeptide to contact a patient sample; and detecting specific binding between the polypeptide and any interacting molecule in the sample to determine whether the subject has cancer.

The invention provides a kit comprising a pharmaceutical composition comprising a pharmaceutically acceptable carrier, an antibody as described above, and instructions for administration into a human or non-human animal.

The invention provides a method for treating uncontrolled proliferative growth in a subject comprising administering a composition comprising an isolated antibody that specifically recognizes, binds to, and/or modulates the biological activity of at least one polypeptide or polynucleotide selected from the Tables, the “non-TM Coords” in Table 3, “Pfam Coords” in Table 4, or the Sequence Listing.

The invention provides a method for treating uncontrolled proliferative growth in a subject comprising administering a modulator to a subject, wherein the modulator binds to or interferes with the activity of at least one polypeptide or polynucleotide selected from the Tables, the “non-TM Coords” in Table 3, “Pfam Coords” in Table 4, or the Sequence Listing. The polypeptide can be phosphatidic acid phosphatase type 2C or COL11A1. The uncontrolled proliferative growth can be a tumor or psoriasis. The tumor can be a lung tumor, a colon tumor, a bladder tumor, a liver tumor, an ovarian tumor, a breast tumor, a kidney tumor, or a pancreatic tumor. The composition can administered, for example, orally, parenterally, by implantation, by inhalation, intranasally, intravenously, intra-arterially, intracardiacally, subcutaneously, intraperitoneally, transdermally, intraventricularly, intracranially, and intrathecally.

The invention yet also provides a method of treating a proliferative disease by providing an antibody composition that comprises a first antibody or fragment thereof that specifically binds to a first epitope of a first polypeptide or a biologically active fragment thereof, wherein the first polypeptide is encoded by a polynucleotide sequence or polypeptide sequence found in Table 1 and/or the Sequence Listing, and administering the antibody composition to a subject in need of such treatment. The antibody composition can further comprise a second antibody that binds specifically to or interferes with the activity of a second epitope of the first polypeptide or to a first epitope of a second polypeptide. The second polypeptide can be chosen from the Sequence Listing and/or Tables.

The invention provides therapeutic agent screening, such as small molecule drug screening; therapeutic applications, such as in the treatment of a variety of diseases and conditions, including, e.g., cancer, proliferative disorders, immune disorders, inflammatory disorders, and other metabolic disorders.

The invention further provides a kit comprising an antibody as described above, and instructions for its use.

The invention yet further provides method of gene therapy, comprising providing a polynucleotide comprising a nucleic acid molecule encoding the antibody, of claim 1, and administering the polynucleotide to a subject in need of such treatment.

The invention provides a method for prophylactically or therapeutically treating a subject by providing a vaccine and administering the vaccine to the subject; wherein the vaccine comprises a polynucleotide or a polypeptide found in the Sequence Listing or Tables, or a fragment thereof, an analogue thereof, or an antagonist thereto. The vaccine can be a cancer vaccine, and the polypeptide can be a cancer antigen. Therapeutic vaccines can be in the form of nucleic acid or polypeptide vaccines, and can be administered alone, such as naked DNA, or can be facilitated, such as via the use of a viral vector, microsomes, or liposomes.

The invention also provides a method of inhibiting transcription or translation of a first polynucleotide encoding a first polypeptide by providing a second polynucleotide that hybridizes to the first polynucleotide, wherein the first polynucleotide comprises a polynucleotide sequence chosen from a polynucleotide or a polypeptide found in the Sequence Listing or Tables, or a fragment thereof, an analogue thereof, or an antagonist thereto, and allowing the first polynucleotide to contact the second polynucleotide. The second polynucleotide can comprise an antisense molecule, a ribozyme, and/or an interfering RNA (iRNA) molecule.

The invention yet also provides a method of treating a proliferative disorder by administering a modulator to a subject in need of such treatment, wherein the modulator binds to a cell surface molecule that is overexpressed in the disorder. The modulator can be an antibody, for example, one that is capable of initiating ADCC.

The invention provides a method of treating a lung tumor in a subject by providing a modulator composition as described above and administering the modulator composition to the subject. The modulator can be an antibody. The antibody can specifically recognize, binds to, or modulate the biological activity of a polypeptide, and the polypeptide can be PAP2C or COL11A1.

The invention provides a method of treating a breast tumor in a subject by providing the modulator composition as described above and administering the modulator composition to the subject. This modulator can be an antibody. The antibody can specifically recognize, bind to, or modulate the biological activity of a polypeptide, and the polypeptide can be PAP2C or COL11A1.

The invention provides a method of treating a colon tumor in a subject by providing a modulator composition as described above and administering the modulator composition to the subject. The modulator can be an antibody. The antibody can specifically recognize, bind to, or modulate the biological activity of the polypeptide. The polypeptide can be PAP2C or COL11A1.

The invention provides a method of treating a liver tumor in a subject by providing a modulator composition as described above and administering the modulator composition to the subject. The modulator can be an antibody. The antibody can specifically recognize, bind to, or modulate the biological activity of the polypeptide. The polypeptide can be PAP2C or COL11A1.

The invention provides a method of treating an ovarian tumor in a subject by providing a modulator composition as described above and administering the modulator composition to the subject. The modulator can be an antibody. The antibody can specifically recognize, bind to, or modulate the biological activity of the polypeptide. The polypeptide can be PAP2C or COL11A1.

The invention provides a method of treating a pancreatic tumor in a subject by providing a modulator composition as described above and administering the modulator composition to the subject. The modulator can be an antibody. The antibody can specifically recognize, bind to, or modulate the biological activity of the polypeptide. The polypeptide can be PAP2C or COL11A1.

The invention provides a method of treating a kidney tumor in a subject by providing a modulator composition as described above and administering the modulator composition to the subject. The modulator can be an antibody. The antibody can specifically recognize, bind to, or modulate the biological activity of the polypeptide. The polypeptide can be PAP2C or COL11A1.

The invention provides a method of treating a stomach tumor in a subject by providing a modulator composition as described above and administering the modulator composition to the subject. The modulator can be an antibody. The antibody can specifically recognize, bind to, or modulate the biological activity of the polypeptide. The polypeptide can be PAP2C or COL11A1.

The invention provides a method of treating a tumor in a subject by providing a modulator composition as described above and administering the modulator composition to the subject. The modulator can be an antibody. The antibody can specifically recognize, bind to, or modulate the biological activity of the polypeptide. The polypeptide can be PAP2C or COL11A1.

The invention provides a method of treating an immune disorder in a subject by providing a modulator composition as described above and administering the modulator composition to the subject. The modulator can be an antibody. The antibody can specifically recognize, bind to, or modulate the biological activity of the polypeptide. The polypeptide can be PAP2C or COL11A1.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications can be made to adapt a particular situation, material, composition of matter, process, process step or steps, to the objective, spirit and scope of the present invention. All such modifications are intended to be within the scope of the claims appended hereto.

### Example 1

### Production of Antibodies to PAP2C

PAP2C can be expressed in vitro in a cell free expression system, using wheat germ lysate or E. coli lysate. Alternatively, PAP2C can be expressed in a baculovirus system (Doerfler, W., Bohm, P., eds. 1987; Luckow, V. and Summers, M. 1988). The expressed protein can be substantially purified (Deutscher, M. P., et al., eds. 1990) and used for injection into mice for production of antibodies. The mice can be normal mice, in which case, the resulting monoclonal antibodies can be made in accordance to conventional techniques, but will be humanized for use in the treatment of humans. The expressed protein can also be used for injection into XenoMouse or other similar mice owned by Abgenix, Inc. (Fremont, Calif., USA), Medarex, Inc. (Princeton, N.J., USA) or Kirin (Japan), which are capable of producing human antibodies.

The expressed protein can also be used to screen for binding with Fab fragments of antibodies displayed on bacteriophages, using phage display libraries, such as is available from Cambridge Antibody Technology (Cambridge, U.K.), MorphoSys (Martinsried/Munich, Germany) or Dyax Corp. (Cambridge, Mass., USA). The Fab fragments that bind the PAP2C polypeptide with high affinity can be validated by immunohistochemistry as binding to tumor tissues. The desired Fab fragment can fused to an appropriate Fc fragment to make a synthetic antibody.

### INDUSTRIAL APPLICABILITY

The compositions and methods of the invention are useful in the diagnosis, treatment, or prevention of proliferative and immune disorders.

### REFERENCES

The specification is most thoroughly understood in light of the following references, all of which are hereby incorporated in their entireties. The disclosures of the patents and other references cited above are also hereby incorporated by reference.
1. Agrawal, S., et al., eds. (1998) Antisense Research and Application (Handbook of Experimental Pharmacology Vol 131). Springer-Verlag New York, Inc.
2. American Cancer Society (2003) Cancer Facts & Figures. (http://www. cancer.org/downloads/STT/CAFF2003PWSecured.pdf).
3. Beigelman, L., et al. (1995) Synthesis of 2′-modified nucleotides and their incorporation into hammerhead ribozymes. Nucleic Acids Res. 23:4434-4442.
4. Chen, S. Y., et al. (1994) Intracellular antibodies as a new class of therapeutic molecule for gene therapy. Hum. Gene Ther. 5:595-601.
5. Choi-Miura, N. H., et al. (1996) Purification and characterization of a novel hyaluronan-binding protein (PHBP) from human plasma: it has three EGF, a kringle and a serine protease domain, similar to hepatocyte growth factor activator. J. Biochem. (Tokyo) 119:1157-1165.
6. Coligan, J. E. et al., eds. (2002) Current Protocols in Immunology, vols. 1-4, including quarterly suppl.) John Wiley and Sons, Inc. New York, N.Y.
7. Deutscher, M. P., et al., eds. (1990) Guide to Protein Purification: Methods in Enzymology. (Methods in Enzymology Series Vol 182. Academic Press.
8. Doerfler, W., Bohm, P., eds. (1987) The Molecular Biology Of Baculoviruses. Springer-Verlag, Inc.
9. Gaudilliere, B., et al. (2002) RNA interference reveals a requirement for MEF2A in activity-dependent neuronal survival. J. Biol. Chem. 277:46,442-46, 446.
10. Gennaro, A., ed. (2000) Remington: The Science and Practice of Pharmacy. 20th ed. Lippincott, Williams, & Wilkins.
11. Gorman, et al. (1982) The Rous sarcoma virus long terminal repeat is a strong promoter when introduced into a variety of eucaryotic cells by DNA-mediated transfection. Proc. Natl. Acad. Sci. 79:6777-6781.
12. Grosschedl, R., Baltimore, D. (1985) Cell-type specificity of immunoglobulin gene expression is regulated by at least three DNA sequence elements. Cell 41:885-897.
13. Grosveld, F., Kollias, G., eds. (1992) Transgenic Animals. 1^{st} ed. Academic Press.
14. Harlow, E., Lane, D., eds. (1988) Antibodies: A Laboratory Manual. Cold Spring Harbor Laboratory.
15. Harlow, E., et al., eds. (1998) Using Antibodies: A Laboratory Manual: Portable Protocol NO. I. Cold Spring Harbor Laboratory.
16. Hartmann, G., Endres, S., eds. (1999) Manual of Antisense Methodology (Perspectives in Antisense Science). 1^{st} ed. Kluwer Law International.
17. Hassanzadeh, G. H. G., et al. (1998) Isolation and characterization of single-chain Fv genes encoding antibodies specific for Drosophila Poxn protein. FEBS Lett. 437:75-80.
18. Hoogenboom, H. R., et al. (1998) Antibody phage display technology and its applications. Immunotechnology 4:1-20.
19. Howard, G. C., Bethell, D. R. (2000) Basic Methods in Antibody Production and Characterization. CRC Press.
20. Jones, P., ed. (1998) Vectors: Expression Systems: Essential Techniques, John Wiley & Son, Ltd.
21. Jost, C. R., et al. (1994) Mammalian expression and secretion of functional single-chain Fv molecules. J. Biol. Chem. 269:26,267-26, 273.
22. Kabat, E. A., Wu T. T. (1991) Identical V region amino acid sequences and segments of sequences in antibodies of different specificities. Relative contributions of VH and VL genes, minigenes, and complementarity-determining regions to binding of antibody-combining sites. J Immunol. 147:1709-1719.
23. Kameyama T, et al. (1996) Identification of plexin family molecules in mice. Biochem. Biophys. Res. Commun. 226:396-402.
24. Kanoh, H., Kai, M., Wada, I. (1999) Molecular characterization of the type 2 phosphatidic acid phosphatase. Chem. Phys. Lipids 98:119-126.
25. Kibbe, A. H., ed. (2000) Handbook of Pharmaceutical Excipients. 3^{rd} ed. Pharmaceutical Press.
26. Kolonin, M. G., Finley, R. L. Jr. (1998) Targeting cyclin-dependent kinases in Drosophila with peptide aptamers. Proc. Natl. Acad. Sci. 95:14, 266-14, 271.
27. Kuisle, O., et al., (1999) Solid phase synthesis of depsides and depsipeptides. Tetrahedron Lett. 40:1203-1206.
28. Lerner, R. A. (1982) Tapping the immunological repertoire to produce antibodies of predetermined specificity. Nature 299:592-596.
29. Liu A. Y., et al. (1987a) Chimeric mouse-human IgG1 antibody that can mediate lysis of cancer cells. Proc. Natl. Acad. Sci. 84:3439-3443.
30. Liu, A. Y., et al. (1987b) Production of a mouse-human chimeric monoclonal antibody to CD20 with potent Fc-dependent biologic activity. J Immunol. 139:3521-26.
31. Lo, N. W., et al. (1998) The expanding beta 4-galactosyltransferase gene family: messages from the databanks. Glycobiology. 8:517-526.
32. Luckow, V., and Summers, M. (1988) Trends in the development of baculovirus expression vectors. Bio/Technology 6:47-55.
33. Milligan, J. F., et al. (1993) Current concepts in antisense drug design. J. Med. Chem. 36:1923-1937.
34. Monfardini, C., et al. (1995) A branched monomethoxypoly(ethylene glycol) for protein modification. Bioconjugate Chem. 6:62-69.
35. Murphy, D., Carter, D. A., eds. (1993) Transgenesis Techniques: Principles and Protocols. Humana Press.
36. Okayama, H., Berg, P. (1983) A cDNA cloning vector that permits expression of cDNA inserts in mammalian cells. Mol. Cell. Biol. 3:280-289.
37. O'Neil, N. J., et al., (2001) RNA-mediated interference as a tool for identifying drug targets. Am. J. Pharmacogenomics 1:45-53.
38. Phillips, M. I., ed. (1999a) Antisense Technology, Part A. Methods in Enzymology Vol. 313. Academic Press, Inc.
39. Phillips, M. I., ed. (1999b) Antisense Technology, Part B. Methods in Enzymology Vol. 314. Academic Press, Inc.
40. Pinkert, C. A., ed. (1994) Transgenic Animal Technology: A Laboratory Handbook. Academic Press.
41. Remington, J. P. (1985) Remington's Pharmaceutical Sciences. 17th ed. Mack Publishing Co.
42. Roberts, R., et al. (1998) Human type 2 phosphatidic acid phosphohydrolases. Substrate specificity of the type 2a, 2b, and 2c enzymes and cell surface activity of the 2a isoform. J. Biol. Chem. 273:22059-22067.
43. Rosok, O., et al. (2000) The C1Chr1 Orf9 gene encodes a putative transmembrane member of a novel protein family. Biochem. Biophys. Res. Commun. 267:855-862.
44. Sambrook, J., Russell, D. W., Sambrook, J. (1989) Molecular Cloning, A Laboratory Manual. 2^{nd} ed. Cold Spring Harbor Laboratory Press.
45. Seki, N., et al. (1997) Characterization of cDNA clones in size-fractionated cDNA libraries from human brain. DNA Res. 4:345-349.
46. Shinnick, T. M., et al. (1983) Synthetic peptide immunogens as vaccines. Ann. Rev. Microbiol. 37:425-446.
47. Stein, C. A., Kreig, A. M., eds. (1998) Applied Antisense Oligonucleotide Technology. Wiley-Liss.
48. Sumiya, J., et al. (1997) Isolation and characterization of the plasma hyaluronan-binding protein (PHBP) gene (HABP2). J. Biochem. (Tokyo) 122:983-990.
49. Sutcliffe, J. G., et al. (1983) Antibodies that react with predetermined sites on proteins. Science 219:660-666.
50. Tamagnone, L., et al. (1999) Plexins are a large family of receptors for transmembrane, secreted, and GPI-anchored semaphorins in vertebrates. Cell 99:71-80.
51. Tan, F., et al. (1997) Sequence of human carboxypeptidase D reveals it to be a member of the regulatory carboxypeptidase family with three tandem active site domains. Biochem. J. 327 (Pt 1):81-87.
52. Tang, D. C., et al. (1992) Genetic immunization is a simple method for eliciting an immune response. Nature 356:152-154.
53. Tomiyasu, M., et al. (2002) Quantification of macrophage migration inhibitory factor mRNA expression in non-small cell lung cancer tissues and its clinical significance. Clin. Cancer Res. 8:3755-3760.
54. Velling, T., et al. (1999) cDNA cloning and chromosomal localization of human alpha(11) integrin. A collagen-binding, I domain-containing, beta(1)-associated integrin alpha-chain present in muscle tissues. J. Biol. Chem. 274:25735-25742.
55. Wagner, R. W., et al. (1996) Potent and selective inhibition of gene expression by an antisense heptanucleotide. Nat. Biotechnol. 14:840-844.
56. Wagner, R. W., et al. (1993) Antisense gene inhibition by oligonucleotides containing C-5 propyne pyrimidines. Science 260:1510-1513.
57. Wang, K. K., et al. (2002) Novel candidate tumor marker genes for lung adenocarcinoma. Oncogene. 21:7598-7604.
58. Xu, C. W., et al. (1997) Cells that register logical relationships among proteins. Proc. Natl. Acad. Sci. (USA) 94:12,473-12,478.
59. Yoshioka H, Ramirez F. (1990) Pro-alpha 1 (XI) collagen. Structure of the amino-terminal propeptide and expression of the gene in tumor cell lines. J. Biol. Chem. 265:6423-6426.
60. Zallipsky, S. (1995) Functionalized poly(ethylene glycols) for preparation of biologically relevant conjugates. Bioconjugate Chem., 6:150-165.
61. Zhidkova, N. I., et al. (1995) Alternative mRNA processing occurs in the variable region of the pro-alpha 11(XI) and pro-alpha 2 (XI) collagen chains. J. Biol. Chem. 270:9486-9493.

### SEQUENCE LISTING

A sequence listing transmittal sheet and a sequence listing in paper format accompanies this application.

### Tables

TABLE 1
Sequence Listing

| | SEQ. ID. | SEQ. ID. NO. | SEQ. ID. | |
|---|---|---|---|---|
| FP ID | NO. (N1) | (P1) | NO. (N0) | Protein ID |
| HG1014556 | SEQ. ID. | SEQ. ID. NO. 4 | SEQ. ID. | NP_003703 |
| | NO. 1 | | NO. 8 | |
| HG1014559 | SEQ. ID. | SEQ. ID. NO. 5 | SEQ. ID. | NP_803545 |
| | NO. 2 | | NO. 9 | |
| HG1014560 | SEQ. ID. | SEQ. ID. NO. 6 | SEQ. ID. | NP_808211 |
| | NO. 3 | | NO. 10 | |
| HG1014558 | | SEQ. ID. NO. 7 | | PAP2domain |

TABLE 2
Annotated Sequences

| FP ID | Protein ID | Annotation |
|---|---|---|
| HG1014563 | 730241:473936 | gi\|730241\|sp\|P39656\|OST4_HUMAN Dolichyl-diphosphooligosaccharide--protein |
| | | glycosyltransferase 48 kDa subunit precursor (Oligosaccharyl transferase 48 kDa subunit) |
| | | (DDOST 48 kDa subunit) |
| HG1014564 | proteinkinase98A:proteinkinase98B | gi\|17975765\|ref\|NP_059145.1\|ephrin receptor EphB2 isoform 1 precursor; developmentally- |
| | | regulated eph-related tyrosine kinase; elk-related tyrosine kinase; eph tyrosine kinase 3 [Homo |
| | | sapiens] |
| HG1014565 | NP_006501:NM_006510 | gi\|5730009\|ref\|NP_006501.1\|ret finger protein isoform alpha; tripartite motif protein TRIM27 |
| | | [Homo sapiens] |
| HG1014566 | 2738927:2738926 | gi\|2738927\|gb\|AAB97675.1\|unknown protein [Homo sapiens] |
| HG1014567 | 3646130:3646129 | gi\|3646130\|emb\|CAA09376.1\|ATP(GTP)-binding protein [Homo sapiens] |
| HG1014568 | 7512502:7512502_genewise | gi\|7512502\|pir\|\|T01371 hypothetical protein 327024.1 - human |
| HG1014569 | 88918:550030 | gi\|88918\|pir\|\|C30127 transmembrane carcinoembryonic antigen 3 precursor - human |
| HG1014570 | 4240243:4240242 | gi\|4240243\|dbj\|BAA74900.1\|KIAA0877 protein [Homo sapiens] |
| HG1014571 | NP_056438:NM_015623 | putative ankyrin-repeat containing protein [Homo sapiens]. |
| HG1014572 | NP_001703:NM_001712 | gi\|19923195\|ref\|NP_001703.2\|carcinoembryonic antigen-related cell adhesion molecule 1 |
| | | (biliary glycoprotein) [Homo sapiens] |
| HG1014573 | NP_003703:NM_003712 | gi\|4505977\|ref\|NP_003703.1\|phosphatidic acid phosphatase type 2C isoform 1; phosphatidic |
| | | acid phosphohydrolase type 2c; type-2 phosphatidic acid phosphatase-gamma [Homo sapiens] |
| HG1014574 | proteinkinase16A:proteinkinase16B | gi\|4501895\|ref\|NP_001096.1\|activin A type I receptor precursor, activin A receptor, type II-like |
| | | kinase 2; hydroxyalkyl-protein kinase [Homo sapiens] |
| HG1014575 | 602434:602433 | gi\|602434\|gb\|AAA86990.1\|GABA/noradrenaline transporter |
| HG1014576 | NP_005177:NM_005186 | gi\|12408656\|ref\|NP_005177.2\|calpain 1, large subunit; calpain, large polypeptide L1; calcium- |
| | | activated neutral proteinase [Homo sapiens] |
| HG1014577 | 3327124:3327123 | gi\|3327124\|dbj\|BAA31630.1\|KIAA0655 protein [Homo sapiens] |
| HG1014578 | NP_001934:NM_001943 | gi\|4503403\|ref\|NP_001934.1\|desmoglein 2 preproprotein; HDGC, included [Homo sapiens] |
| HG1014579 | NP_002417:NM_002426 | gi\|4505207\|ref\|NP_002417.1\|matrix metalloproteinase 12 preproprotein; macrophage |
| | | metalloelastase; macrophage elastase |
| | | [Homo sapiens]_gi\|435970\|gb\|AAA58658.1\|metalloproteinase |
| HG1014580 | NP_002236:NM_002245 | gi\|4504847\|ref\|NP_002236.1\|potassium channel, subfamily K, member 1; potassium inwardly- |
| | | rectifying channel, subfamily K, member 1; potassium channel, subfamily K, member 1 |
| | | (TWIK-1) [Homo sapiens] |
| HG1014581 | 3882213:3882212 | gi\|3882213\|dbj\|BAA34466.1\|KIAA0746 protein [Homo sapiens] |
| HG1014582 | 2439970:2439969 | gi\|2439970\|gb\|AAB71756.1\|multidrug resistance-associated protein homolog [Homo sapiens] |
| HG1014583 | NP_005859:NM_005868 | gi\|5031611\|ref\|NP_005859.1\|BET1 homolog; Golgi vesicular membrane trafficking protein p18; |
| | | Bet1p homolog [Homo sapiens]_gi\|27805424\|sp\|O15155\|BET1_HUMAN BET1 homolog |
| | | (Golgi vesicular membrane trafficking protein p18) |
| | | (hBET1)_gi\|2253426\|gb\|AAB62941.1\|Bet1p homolog [Homo sapiens] |
| HG1014584 | NP_005778:NM_005787 | gi\|5031953\|ref\|NP_005778.1\|asparagine-linked glycosylation 3 homolog (yeast, alpha-1,3- |
| | | mannosyltransferase); Not56 (D. melanogaster)-like protein [Homo sapiens] |
| HG1014585 | 887368:887367 | gi\|887368\|gb\|AAC42003.1\|ORF; putative |
| HG1014586 | NP_055688:NM_014873 | gi\|7661996\|ref\|NP_055688.1\|KIAA0205 gene product [Homo sapiens] |
| HG1014587 | 7513004:3043577 | gi\|7513004\|pir\|\|T00073 hypothetical protein KIAA0527 - human (fragment) |
| HG1014588 | 20521660:20521659 | gi\|20521660\|dbj\|BAA34508.2\|KIAA0788 protein [Homo sapiens] |
| HG1014589 | 12230553:1665780 | gi\|12230553\|sp\|Q92545\|RW1_HUMAN RW1 protein |
| HG1014590 | NP_059984:NM_017514 | gi\|8923793\|ref\|NP_059984.1\|SEX gene [Homo sapiens] |
| HG1014591 | NP_002831:NM_002840 | gi\|4506311\|ref\|NP_002831.1\|protein tyrosine phosphatase, receptor type, F isoform 1 precursor; |
| | | protein tyrosine phosphatase, receptor type, F polypeptide; receptor-linked protein-tyrosine |
| | | phosphatase LAR; leukocyte antigen-related tyrosine phosphatase; LCA-homolog; leukocyte |
| | | antigen-related (LAR) PTP receptor [Homo sapiens] |
| HG1014592 | 3043698:3043697 | KIAA0587 protein [Homo sapiens]. |
| HG1014593 | 14133205:14133204 | gi\|14133205\|dbj\|BAA32311.2\|KIAA0466 protein [Homo sapiens] |
| HG1014594 | NP_055453:NM_014638 | KIAA0450 gene product [Homo sapiens]. |
| HG1014595 | NP_064422:NM_020038 | gi\|9955974\|ref\|NP_064422.1\|ATP-binding cassette, sub-family C, member 3 isoform MRP3B; |
| | | canicular multispecific organic anion transporter [Homo sapiens] |
| HG1014596 | 1580781:1580780 | gi\|1580781\|gb\|AAB09603.1\|beige-like protein [Homo sapiens] |
| HG1014597 | 2136093:403386 | gi\|2136093\|pir\|\|A48280 receptor tyrosine kinase - human |
| HG1014598 | NP_005119:NM_005128 | gi\|4826653\|ref\|NP_005119.1\|pad-1-like [Homo sapiens] |
| HG1014599 | 559330:559329 | gi\|559330\|dbj\|BAA07526.1\|KIAA0077 [Homo sapiens] |
| HG1014600 | 1665787:1665786 | gi\|1665787\|dbj\|BAA13390.1\|Similar to a C. elegans protein encoded in cosmid C52E12 |
| | | (U50135) [Homo sapiens] |
| HG1014601 | NP_003307:NM_003316 | gi\|21359841\|ref\|NP_003307.2\|tetratricopeptide repeat domain 3; tetratricopeptide repeat |
| | | protein 3 (TPR repeat protein D) [Homo sapiens] |
| HG1014602 | NP_055098:NM_014283 | gi\|7656940\|ref\|NP_055098.1\|chromosome 1 open reading frame 9; membrane protein CH1 |
| | | [Homo sapiens] |
| HG1014603 | 21903712:22004648 | gi\|21903712\|gb\|AAC51775.2\|carboxypeptidase D [Homo sapiens] |
| HG1014604 | 403460:403459 | gi\|403460\|gb\|AAA36776.1\|transformation-related protein |
| HG1014605 | 20140021:1888315 | gi\|20140021\|sp\|Q12884\|SEPR_HUMAN Seprase (Fibroblast activation protein alpha) (Integral |
| | | membrane serine protease) (170-kDa melanoma membrane-bound gelatinase) |
| HG1014606 | 2996578:2996577 | gi\|2996578\|emb\|CAA12176.1\|glucosyltransferase [Homo sapiens] |
| HG1014607 | 729008:306474 | gi\|729008\|sp\|Q08345\|DDR1_HUMAN Epithelial discoidin domain receptor 1 precursor |
| | | (Tyrosine-protein kinase CAK) (Cell adhesion kinase) (Tyrosine kinase DDR) (Discoidin |
| | | receptor tyrosine kinase) (TRK E) (Protein-tyrosine kinase RTK 6) (CD167a antigen) |
| HG1014608 | NP_001296:NM_001305 | gi\|4502877\|ref\|NP_001296.1\|claudin 4; Clostridium perfringens enterotoxin receptor; |
| | | Clostridium perfringens enterotoxin receptor 1 [Homo sapiens] |
| HG1014609 | NP_066192:NM_020982 | gi\|11141861\|ref\|NP_066192.1\|claudin 9 [Homo sapiens] |
| HG1014610 | NP_006293:NM_006302 | gi\|5453662\|ref\|NP_006293.1\|mannosyl-oligosaccharide glucosidase; processing A-glucosidase I |
| | | [Homo sapiens] |
| HG1014611 | 4691263:4557422 | gi\|4691263\|emb\|CAB41571.1\|dJ738P15.2.1 (ectonucleoside triphosphate diphosphohydrolase 6 |
| | | (putative function), isoform 1) [Homo sapiens] |
| HG1014612 | NP_006806:NM_006815 | gi\|5803149\|ref\|NP_006806.1\|coated vesicle membrane protein [Homo sapiens] |
| HG1014613 | NP_036380:NM_012248 | gi\|15011844\|ref\|NP_036380.2\|selenophosphate synthetase 2; selenide, water dikinase 2; |
| | | selenium donor protein 2; selenophosphate synthase [Homo sapiens] |
| HG1014614 | 5459516:5459515 | gi\|5459516\|dbj\|BAA82407.1\|phosphatidylethanolamine N-methyltransferase [Homo sapiens] |
| HG1014615 | proteinkinase99A:proteinkinase99B | protein kinase EphB3 |
| HG1014616 | NP_055557:NM_014742 | gi\|7662028\|ref\|NP_055557.1\|transmembrane 9 superfamily protein member 4 [Homo sapiens] |
| HG1014617 | 4009517:4009516 | gi\|4009517\|gb\|AAC95470.1\|type 2 iodothyronine deiodinase [Homo sapiens] |
| HG1014618 | 1220309:1220308 | gi\|1220309\|gb\|AAA91834.1\|gamma-glutamic carboxylase |
| HG1014619 | NP_005679:NM_005688 | gi\|5032101\|ref\|NP_005679.1\|ATP-binding cassette, sub-family C, member 5; canalicular |
| | | multispecific organic anion transporter C [Homo sapiens] |
| HG1014620 | NP_004985:NM_004994 | gi\|4826836\|ref\|NP_004985.1\|matrix metalloproteinase 9 preproprotein; 92 kD type IV |
| | | collagenase; matrix metalloproteinase 9 (gelatinase B, 92 kD gelatinase, 92 kD type IV |
| | | collagenase); gelatinase B; macrophage gelatinase; type V collagenase [Homo sapiens] |
| HG1014621 | 1478281:1478280 | gi\|1478281\|gb\|AAC50629.1\|neutral amino acid transporter B |
| HG1014622 | NP_055759:NM_014944 | gi\|7662374\|ref\|NP_055759.1\|calsyntenin 1 [Homo sapiens] |
| HG1014623 | NP_066925:NM_021102 | gi\|10863909\|ref\|NP_066925.1\|serine protease inhibitor, Kunitz type, 2; placental bikunin; |
| | | Kunitz-type serine protease inhibitor; hepatocyte growth factor activator inhibitor type 2 |
| | | [Homo sapiens] |
| HG1014624 | NP_000201:NM_000210 | gi\|4557675\|ref\|NP_000201.1\|integrin alpha chain, alpha 6 [Homo sapiens] |
| HG1014625 | NP_006661:NM_006670 | gi\|5729718\|ref\|NP_006661.1\|5T4 oncofetal trophoblast glycoprotein; 5T4-antigen |
| | | [Homo sapiens] |
| HG1014626 | NP_000204:NM_000213 | gi\|21361207\|ref\|NP_000204.2\|integrin, beta 4 [Homo sapiens] |
| HG1014627 | NP_005767:NM_005776 | gi\|5031639\|ref\|NP_005767.1\|cornichon-like [Homo sapiens] |
| HG1014628 | 3288487:3288486 | gi\|3288487\|emb\|CAA75875.1\|COL1A1 and PDGFB fusion transcript [Homo sapiens] |
| HG1014629 | 13124728:2285960 | gi\|13124728\|sp\|P78334\|GAE_HUMAN Gamma-aminobutyric-acid receptor epsilon subunit |
| | | precursor (GABA(A) receptor) |
| HG1014630 | 239160:239159 | gi\|239160\|gb\|AAB20355.1\|integrin alpha 6B [Homo sapiens] |
| HG1014631 | NP_003701:NM_003710 | gi\|4504329\|ref\|NP_003701.1\|hepatocyte growth factor activator inhibitor 1 isoform 2 precursor; |
| | | hepatocyte growth factor activator inhibitor 1; Kunitz-type protease inhibitor 1 [Homo sapiens] |
| HG1014632 | NP_002345:NM_002354 | gi\|4505059\|ref\|NP_002345.1\|tumor-associated calcium signal transducer 1 precursor; membrane |
| | | component, chromosome 4, surface marker (35 kD glycoprotein); MK-1 antigen; antigen |
| | | identified by monoclonal antibody AUA1; human epithelial glycoprotein-2 |
| | | [Homo sapiens] |
| HG1014633 | NP_036451:NM_012319 | gi\|12751475\|ref\|NP_036451.2\|solute carrier family 39 (zinc transporter), member 6; LIV-1 |
| | | protein, estrogen regulated; solute carrier family 39 (metal ion transporter), member 6 |
| | | [Homo sapiens] |
| HG1014634 | NP_002241:NM_002250 | gi\|4504859\|ref\|NP_002241.1\|intermediate conductance calcium-activated potassium channel |
| | | protein 1; putative erythrocyte intermediate conductance calcium-activated potassium Gardos |
| | | channel [Homo sapiens] |
| HG1014635 | 3387977:3387976 | gi\|3387977\|gb\|AAC28653.1\|ABC transporter [Homo sapiens] |
| HG1014636 | NP_001297:NM_001306 | gi\|4502875\|ref\|NP_001297.1\|claudin 3; Clostridium perfringens enterotoxin receptor 2; rat |
| | | ventral prostate.1-like protein; claudin-3; CPE-receptor 2 [Homo sapiens] |
| HG1014637 | 3132896:3132895 | gi\|3132896\|gb\|AAC39733.1\|beta-1,4-galactosyltransferase [Homo sapiens] |
| HG1014638 | 20521832:20521831 | gi\|20521832\|dbj\|BAA09768.3\|KIAA0147 protein [Homo sapiens] |
| HG1014639 | NP_003830:NM_003839 | gi\|4507565\|ref\|NP_003830.1\|tumor necrosis factor receptor superfamily, member 11a precursor; |
| | | activator of NFKB; receptor activator of nuclear factor-kappa B; osteoclast differentiation factor |
| | | receptor [Homo sapiens] |
| HG1014640 | NP_001100:NM_001109 | gi\|4557253\|ref\|NP_001100.1\|a disintegrin and metalloproteinase domain 8 precursor [Homo |
| | | sapiens] |
| HG1014641 | NP_055080:NM_014265 | gi\|7656863\|ref\|NP_055080.1\|a disintegrin and metalloproteinase domain 28 isoform 1 |
| | | preproprotein [Homo sapiens] |
| HG1014642 | NP_005497:NM_005506 | gi\|5031631\|ref\|NP_005497.1\|scavenger receptor class B, member 2; lysosomal integral |
| | | membrane protein II; CD36 antigen (collagen type I receptor, thrombospondin receptor)-like 2; |
| | | 85 kDa lysosomal sialoglycoprotein scavenger receptor class B, member 2 [Homo sapiens] |
| HG1014643 | NP_006685:NM_006694 | gi\|5729889\|ref\|NP_006685.1\|jumping translocation breakpoint; PAR protein [Homo sapiens] |
| HG1014644 | 4456467:4456466 | gi\|4456467\|emb\|CAB37294.1\|TM7XN1 protein [Homo sapiens] |
| HG1014645 | NP_002217:NM_002226 | gi\|21704277\|ref\|NP_002217.3\|jagged 2 isoform a precursor [Homo sapiens] |
| HG1014646 | NP_003769:NM_003778 | gi\|9994175\|ref\|NP_003769.1\|UDP-Gal:betaGlcNAc beta 1,4-galactosyltransferase 4; beta-N- |
| | | acetylglucosaminyl-glycolipid beta-1,4-galactosyltransferase 4 [Homo sapiens] |
| HG1014647 | 1504030:1504029 | gi\|1504030\|dbj\|BAA13214.1\|similar to a C. elegans protein encoded in cosmid K12D12(Z49069) |
| | | [Homo sapiens] |
| HG1014692 | NP_068547:NM_021777 | gi\|11496994\|ref\|NP_068547.1\|a disintegrin and metalloproteinase domain 28 isoform 3 |
| | | preproprotein [Homo sapiens] |
| HG1014693 | NP_068548:NM_021778 | gi\|11496996\|ref\|NP_068548.1\|a disintegrin and metalloproteinase domain 28 isoform 2 |
| | | preproprotein [Homo sapiens] |
| HG1014694 | NP_068819:NM_021984 | gi\|12707554\|ref\|NP_068819.1\|gamma-aminobutyric acid (GABA) A receptor, epsilon isoform 2 |
| | | [Homo sapiens] |
| HG1014695 | NP_068822:NM_021987 | gi\|12707556\|ref\|NP_068822.1\|gamma-aminobutyric acid (GABA) A receptor, epsilon isoform 3 |
| | | [Homo sapiens] |
| HG1014696 | NP_068830:NM_021990 | gi\|12707558\|ref\|NP_068830.1\|gamma-aminobutyric acid (GABA) A receptor, epsilon isoform 2 |
| | | [Homo sapiens] |
| HG1014697 | NP_076984:NM_024079 | gi\|13129070\|ref\|NP_076984.1\|asparagine-linked glycosylation 8 homolog (yeast, alpha-1,3- |
| | | glucosyltransferase) [Homo sapiens] |
| HG1014698 | NP_079327:NM_025051 | gi\|13376580\|ref\|NP_079327.1\|hypothetical protein FLJ23022 [Homo sapiens] |
| HG1014699 | NP_108648:NM_030658 | putative ankyrin-repeat containing protein [Homo sapiens] |
| HG1014700 | NP_085076:NM_030587 | gi\|13929465\|ref\|NP_085076.1\|UDP-Gal:betaGlcNAc beta 1,4-galactosyltransferase 2 isoform a; |
| | | beta-4-GalT2; beta-N-acetylglucosaminyl-glycolipid beta-1,4-galactosyltransferase 2 [Homo |
| | | sapiens] |
| HG1014701 | NP_055954:NM_015139 | gi\|14028875\|ref\|NP_055954.1\|solute carrier family 35 (UDP-glucuronic acid/UDP-N- |
| | | acetylgalactosamine dual transporter), member D1; UDP-glucuronic acid/UDP-N- |
| | | acetylgalactosamine dual transporter [Homo sapiens] |
| HG1014702 | NP_009197:NM_007266 | gi\|14149629\|ref\|NP_009197.1\|XPA binding protein 1; MBD2 interactor protein; putative |
| | | ATP(GTP)-binding protein [Homo sapiens] |
| HG1014703 | NP_112212:NM_030950 | gi\|15011933\|ref\|NP_112212.1\|ret finger protein isoform beta; tripartite motif protein TRIM27 |
| | | [Homo sapiens] |
| HG1014704 | NP_073572:NM_022735 | gi\|15826852\|ref\|NP_073572.2\|golgi complex associated protein 1; golgi resident protein GCP60; |
| | | peripherial benzodiazepine receptor associated protein; golgi phosphoprotein 1; PBR associated |
| | | protein; golgi complex associated protein 1, 60 kDa; PKA (RIalpha)-associated protein [Homo |
| | | sapiens] |
| HG1014705 | NP_079461:NM_025185 | putative ankyrin-repeat containing protein [Homo sapiens] |
| HG1014706 | NP_006717:NM_006726 | gi\|16904381\|ref\|NP_006717.1\|LPS-responsive vesicle trafficking, beach and anchor containing; |
| | | vesicle trafficking, beach and anchor containing; cell division cycle 4-like [Homo sapiens] |
| HG1014707 | NP_004434:NM_004443 | gi\|17975768\|ref\|NP_004434.2\|ephrin receptor EphB3 precursor; EPH-like tyrosine kinase-2; |
| | | human embryo kinase 2 [Homo sapiens] |
| HG1014708 | NP_056171:NM_015356 | gi\|18141297\|ref\|NP_056171.1\|scribble [Homo sapiens] |
| HG1014709 | NP_001845:NM_001854 | gi\|18375518\|ref\|NP_001845.2\|alpha 1 type XI collagen isoform A preproprotein; collagen XI, |
| | | alpha-1 polypeptide [Homo sapiens] |
| HG1014710 | NP_569707:NM_130440 | gi\|18860896\|ref\|NP_569707.1\|protein tyrosine phosphatase, receptor type, F isoform 2 |
| | | precursor; protein tyrosine phosphatase, receptor type, F polypeptide; receptor-linked protein- |
| | | tyrosine phosphatase LAR; leukocyte antigen-related tyrosine phosphatase; LCA-homolog; |
| | | leukocyte antigen-related (LAR) PTP receptor [Homo sapiens] |
| HG1014711 | NP_005673:NM_005682 | gi\|19923768\|ref\|NP_005673.2\|G protein-coupled receptor 56; EGF-TM7-like [Homo sapiens] |
| HG1014712 | NP_005207:NM_005216 | gi\|20070197\|ref\|NP_005207.2\|dolichyl-diphosphooligosaccharide-protein glycosyltransferase |
| | | [Homo sapiens] |
| HG1014713 | NP_004433:NM_004442 | gi\|21396504\|ref\|NP_004433.2\|ephrin receptor EphB2 isoform 2 precursor; developmentally- |
| | | regulated eph-related tyrosine kinase; elk-related tyrosine kinase; eph tyrosine kinase 3 [Homo |
| | | sapiens] |
| HG1014714 | NP_660142:NM_145159 | gi\|21704279\|ref\|NP_660142.1\|jagged 2 isoform b precursor [Homo sapiens] |
| HG1014715 | NP_001295:NM_001304 | gi\|22202611\|ref\|NP_001295.2\|carboxypeptidase D precursor [Homo sapiens] |
| HG1014716 | NP_680477:NM_148172 | gi\|22538478\|ref\|NP_680477.1\|phosphatidylethanolamine N-methyltransferase isoform 1 [Homo |
| | | sapiens] |
| HG1014717 | NP_680478:NM_148173 | gi\|22538480\|ref\|NP_680478.1\|phosphatidylethanolamine N-methyltransferase isoform 2 [Homo |
| | | sapiens] |
| HG1014718 | NP_054733:NM_014014 | gi\|40217847\|ref\|NP_054733.2\|U5 snRNP-specific profein, 200-KD [Homo |
| | | sapiens] |
| HG1014719 | NP_803545:NM_177526 | gi\|29171745\|ref\|NP_803545.1\|phosphatidic acid phosphatase type 2C isoform 2; phosphatidic |
| | | acid phosphohydrolase type 2c; type-2 phosphatidic acid phosphatase-gamma [Homo sapiens] |
| HG1014720 | NP_808211:NM_177543 | gi\|29171747\|ref\|NP_808211.1\|phosphatidic acid phosphatase type 2C isoform 3; phosphatidic |
| | | acid phosphohydrolase type 2c; type-2 phosphatidic acid phosphatase-gamma [Homo sapiens] |
| HG1014721 | NP_003771:NM_003780 | gi\|4502347\|ref\|NP_003771.1\|UDP-Gal:betaGlcNAc beta 1,4-galactosyltransferase 2 isoform b; |
| | | beta-4-GalT2; beta-N-acetylglucosaminyl-glycolipid beta-1,4-galactosyltransferase 2 [Homo |
| | | sapiens] |
| HG1014722 | NP_000079:NM_000088 | gi\|4502945\|ref\|NP_000079.1\|alpha 1 type I collagen preproprotein; Collagen I, alpha-1 |
| | | polypeptide; osteogenesis imperfecta type IV; collagen of skin, tendon and bone, alpha-1 chain |
| | | [Homo sapiens] |
| HG1014723 | NP_001533:NM_001542 | gi\|4504627\|ref\|NP_001533.1\|immunoglobulin superfamily, member 3; immunoglobin |
| | | superfamily, member 3 [Homo sapiens] |
| HG1014724 | NP_001238:NM_001247 | gi\|4557423\|ref\|NP_001238.1\|ectonucleoside triphosphate diphosphohydrolase 6; CD39-like 2; |
| | | interleukin 6 signal transducer-2 [Homo sapiens] |
| HG1014725 | NP_004952:NM_004961 | gi\|4826738\|ref\|NP_004952.1\|gamma-aminobutyric acid (GABA) A receptor, epsilon isoform 1 |
| | | precursor [Homo sapiens] |
| HG1014726 | NP_038464:NM_013436 | gi\|7305303\|ref\|NP_038464.1\|NCK-associated protein 1 [Homo sapiens] |
| HG1014727 | NP_054644:NM_013989 | gi\|7549803\|ref\|NP_054644.1\|deiodinase, iodothyronine, type II; thyroxine deiodinase, type II |
| | | [Homo sapiens] |
| HG1014728 | NP_054699:NM_013993 | gi\|7669483\|ref\|NP_054699.1\|discoidin receptor tyrosine kinase isoform a; PTK3A protein |
| | | tyrosine kinase 3A; cell adhesion kinase; epithelial discoidin domain receptor 1; neurotrophic |
| | | tyrosine kinase, receptor, type 4; neuroepithelial tyrosine kinase; mammarian carcinoma kinase |
| | | 10 [Homo sapiens] |
| HG1014729 | NP_054700:NM_013994 | gi\|7669485\|ref\|NP_054700.1\|discoidin receptor tyrosine kinase isoform c; PTK3A protein |
| | | tyrosine kinase 3A; cell adhesion kinase; epithelial discoidin domain receptor 1; neurotrophic |
| | | tyrosine kinase, receptor, type 4; neuroepithelial tyrosine kinase; mammarian carcinoma kinase |
| | | 10 [Homo sapiens] |
| HG1014730 | NP_057311:NM_016227 | gi\|7705322\|ref\|NP_057311.1\|membrane protein CH1 [Homo sapiens] |
| HG1014731 | NP_057725:NM_016641 | gi\|7706617\|ref\|NP_057725.1\|membrane interacting protein of RGS16 [Homo sapiens] |
| HG1014732 | NP_005680:NM_005689 | gi\|9955963\|ref\|NP_005680.1\|ATP-binding cassette, sub-family B, member 6 [Homo sapiens] |
| HG1014733 | NP_003777:NM_003786 | gi\|9955970\|ref\|NP_003777.2\|ATP-binding cassette, sub-family C, member 3 isoform MRP3; |
| | | canicular multispecific organic anion transporter [Homo sapiens] |
| HG1014734 | NP_064421:NM_020037 | gi\|9955972\|ref\|NP_064421.1\|ATP-binding cassette, sub-family C, member 3 isoform MRP3A; |
| | | canicular multispecific organic anion transporter [Homo sapiens] |
| HG1014735 | 10047349:10047348 | KIAA1636 protein [Homo sapiens] |
| HG1014736 | 10435899:10435898 | gi\|10435899\|dbj\|BAB14698.1\|unnamed protein product [Homo sapiens] |
| HG1014737 | 10438061:10438060 | gi\|10438061\|dbj\|BAB15159.1\|unnamed protein product [Homo sapiens] |
| HG1014738 | 10443048:4826835 | gi\|10443048\|emb\|CAC10459.1\|bA465L10.4 (matrix metalloproteinase 9 (gelatinase B, 92 kD |
| | | gelatinase, 92 kD type IV collagenase) (CLG4B)) [Homo sapiens] |
| HG1014739 | 10863065:10863064 | gi\|10863065\|dbj\|BAB16838.1\|type II iodothyronine deiodinase [Homo sapiens] |
| HG1014740 | 10863067:10863066 | gi\|10863067\|dbj\|BAB16839.1\|type II iodothyronine deiodinase [Homo sapiens] |
| HG1014741 | 11245444:11245443 | gi\|11245444\|gb\|AAG33617.1\|ATP-binding cassette half-transporter [Homo sapiens] |
| HG1014742 | 11245446:11245443 | gi\|11245446\|gb\|AAG33618.1\|ATP-binding cassette half-transporter [Homo sapiens] |
| HG1014743 | 12082644:12082643 | gi\|12082644\|gb\|AAG48559.1\|beige-like protein [Homo sapiens] |
| HG1014744 | 12275809:12275808 | gi\|12275809\|gb\|AAG50147.1\|beta-1,4-galactosyltransferase [Homo sapiens] |
| HG1014745 | 12314010:24797104 | gi\|12314010\|emb\|CAC10350.1\|dJ74M1.1.1 (tyrosine kinase isoform 1) [Homo sapiens] |
| HG1014746 | 12314011:17975764 | gi\|12314011\|emb\|CAC10351.1\|dJ74M1.1.2 (tyrosine kinase isosform 2) [Homo sapiens] |
| HG1014747 | 12653567:12653566 | gi\|12653567\|gb\|AAH00557.1\|Phosphatidylethanolamine N-methyltransferase, isoform 1 [Homo |
| | | sapiens] |
| HG1014748 | 12697587:12697586 | gi\|12697587\|dbj\|BAB21594.1\|type II iodothyronine deiodinase [Homo sapiens] |
| HG1014749 | 12803155:12803154 | selenophosphate synthetase 2 [Homo sapiens] |
| HG1014750 | 12803915:12803914 | Similar to glucosidase I [Homo sapiens] |
| HG1014751 | 13279206:13279205 | gi\|13279206\|gb\|AAH04313.1\|ALG3 protein [Homo sapiens] |
| HG1014752 | 13325454:13325453 | gi\|13325454\|gb\|AAH04523.1\|UDP-Gal:betaGlcNAc beta 1,4-galactosyltransferase 4 [Homo |
| | | sapiens] |
| HG1014753 | 13517342:7705321 | gi\|13517342\|gb\|AAK28742.1\|membrane protein CH1 [Homo sapiens] |
| HG1014754 | 13517410:7705321 | gi\|13517410\|gb\|AAK28776.1\|membrane protein CH1 [Homo sapiens] |
| HG1014755 | 13898643:13898642 | gi\|13898643\|gb\|AAK48842.1\|discoidin domain receptor DDR1d [Homo sapiens] |
| HG1014756 | 13898645:13898644 | gi\|13898645\|gb\|AAK48843.1\|discoidin domain receptor DDR1e [Homo sapiens] |
| HG1014757 | 14043169:14043168 | gi\|14043169\|gb\|AAH07572.1\|Unknown (protein for IMAGE: 3030210) [Homo sapiens] |
| HG1014758 | 14043179:14043178 | gi\|14043179\|gb\|AAH07577.1\|Unknown (protein for IMAGE: 3139787) [Homo sapiens] |
| HG1014759 | 14043430:14043429 | gi\|14043430\|gb\|AAH07705.1\|Serine protease inhibitor, Kunitz type, 2 [Homo sapiens] |
| HG1014760 | 14249879:14249878 | Unknown (protein for IMAGE: 3343159) [Homo sapiens] |
| HG1014761 | 14250593:14250592 | gi\|14250593\|gb\|AAH08751.1\|Calpain 1, large subunit [Homo sapiens] |
| HG1014762 | 14550482:14550481 | Unknown (protein for IMAGE: 3936863) [Homo sapiens] |
| HG1014763 | 14602901:14602900 | Unknown (protein for IMAGE: 4123572) [Homo sapiens] |
| HG1014764 | 14724070:22042187 | similar to KIAA0077 [Homo sapiens] |
| HG1014765 | 14726864:14726863 | similar to KIAA0377 gene product [Homo sapiens] |
| HG1014766 | 15029376:15029375 | gi\|15029376\|gb\|AAK81862.1\|potassium intermediate/small conductance calcium-activated |
| | | channel, subfamily N, member 4 [Homo sapiens] |
| HG1014767 | 15214801:15214800 | gi\|15214801\|gb\|AAH12535.1\|LRBA protein [Homo sapiens] |
| HG1014768 | 15214917:15214916 | gi\|15214917\|gb\|AAH12595.1\|BET1 protein [Homo sapiens] |
| HG1014769 | 15559191:9955969 | gi\|15559191\|emb\|CAC69553.1\|multidrug resistance associated protein [Homo sapiens] |
| HG1014770 | 15680237:15680236 | gi\|15680237\|gb\|AAH14473.1\|CEACAM1 protein [Homo sapiens] |
| HG1014771 | 15779135:15779134 | Unknown (protein for IMAGE: 3503007) [Homo sapiens] |
| HG1014772 | 15929829:15929828 | gi\|15929829\|dbj\|AAH15334.1\|Unknown (protein for IMAGE: 4391654) [Homo sapiens] |
| HG1014773 | 1632766:1632765 | gi\|1632766\|gb\|BAA12303.1\|TPRDIII [Homo sapiens] |
| HG1014774 | 16552593:16552592 | gi\|16552593\|dbj\|BAB71347.1\|unnamed protein product [Homo sapiens] |
| HG1014775 | 1688260:4505206 | gi\|1688260\|gb\|AAB36943.1\|metalloelastase [Homo sapiens] |
| HG1014776 | 1747371:1747370 | gi\|1747371\|gb\|CAA68914.1\|putative GABA-gated chloride channel [Homo sapiens] |
| HG1014777 | 179629:179624 | gi\|179629\|gb\|AAA52289.1\|pro-alpha-1 collagen type 1 [Homo sapiens] |
| HG1014778 | 179630:22328091 | gi\|179630\|gb\|AAA52290.1\|pro-alpha-1 collagen type 1 [Homo sapiens] |
| HG1014779 | 179631:179626 | gi\|179631\|gb\|AAA52291.1\|pro-alpha-1 collagen type 1 [Homo sapiens] |
| HG1014780 | 18027796:18027795 | gi\|18027796\|gb\|AAL55859.1\|unknown [Homo sapiens] |
| HG1014781 | 18044628:18044627 | gi\|18044628\|gb\|AAH19679.1\|Unknown (protein for IMAGE: 4932488) [Homo sapiens] |
| HG1014782 | 18676646:18676645 | gi\|18676646\|dbj\|BAB84975.1\|FLJ00222 protein [Homo sapiens] |
| HG1014783 | 1888409:22328091 | gi\|1888409\|emb\|CAA67261.1\|collagen type I alpha 1 [Homo sapiens] |
| HG1014784 | 19684107:19684106 | gi\|19684107\|gb\|AAH25980.1\|Ectonucleoside triphosphate diphosphohydrolase 6 |
| | | (putative function) [Homo sapiens] |
| HG1014785 | 19913138:20130436 | gi\|19913138\|emb\|CAD19636.1\|glucosidase I [Homo sapiens] |
| HG1014786 | 20521698:20521697 | gi\|20521698\|dbj\|BAA76777.2\|KIAA0933 protein [Homo sapiens] |
| HG1014787 | 20540895:20540894 | similar to CG11943-PB [Homo sapiens] |
| HG1014788 | 20541809:20541808 | similar to KIAA0877 protein [Homo sapiens] |
| HG1014789 | 21104416:21104415 | gi\|21104416\|dbj\|BAB93478.1\|dolichyl-diphosphooligosaccharide-protein glycosyltransferase |
| | | [Homo sapiens] |
| HG1014790 | 21434741:21434740 | gi\|21434741\|gb\|AAM53530.1\|beige-like protein; CDC4L protein [Homo sapiens] |
| HG1014791 | 21706696:21706695 | gi\|21706696\|gb\|AAH33902.1\|CLSTN1 protein [Homo sapiens] |
| HG1014792 | 21739637:21739636 | gi\|21739637\|emb\|CAD38864.1\|hypothetical protein [Homo sapiens] |
| HG1014793 | 21748877:21748876 | gi\|21748877\|dbj\|BAC03499.1\|unnamed protein product [Homo sapiens] |
| HG1014794 | 21750497:21750496 | gi\|21750497\|dbj\|BAC03787.1\|unnamed protein product [Homo sapiens] |
| HG1014795 | 21752841:21752840 | gi\|21752841\|dbj\|BAC04245.1\|unnamed protein product [Homo sapiens] |
| HG1014796 | 21757691:21757690 | gi\|21757691\|dbj\|BAC05175.1\|unnamed protein product [Homo sapiens] |
| HG1014797 | 21929079:19923767 | gi\|21929079\|dbj\|BAC06124.1\|seven transmembrane helix receptor [Homo sapiens] |
| HG1014798 | 219495:219494 | gi\|219495\|dbj\|BAA02063.1\|biliary glycoprotein [Homo sapiens] |
| HG1014799 | 21961497:21961496 | Similar to golgi complex associated protein 1, 60 kDa [Homo sapiens] |
| HG1014800 | 2197067:2197066 | gi\|2197067\|gb\|AAB61285.1\|Jagged 2 [Homo sapiens] |
| HG1014801 | 22044017:22044016 | similar to KIAA0527 protein [Homo sapiens] |
| HG1014802 | 22328092:22328091 | gi\|22328092\|gb\|AAH36531.1\|Alpha 1 type I collagen preproprotein [Homo sapiens] |
| HG1014803 | 22532481:4826835 | gi\|22532481\|gb\|AAM97934.1\|matrix metalloproteinase 9 (gelatinase B, 92 kD gelatinase, 92 kD |
| | | type IV collagenase) [Homo sapiens] |
| HG1014804 | 2270923:33910 | gi\|2270923\|gb\|AAC51632.1\|beta4-integrin [Homo sapiens] |
| HG1014805 | 2270924:21361206 | gi\|2270924\|gb\|AAC51633.1\|beta4-integrin [Homo sapiens] |
| HG1014806 | 2270925:33956 | gi\|2270925\|gb\|AAC51634.1\|beta4-integrin [Homo sapiens] |
| HG1014807 | 2285958:2285960 | gi\|2285958\|emb\|CAA70903.1\|GABRE [Homo sapiens] |
| HG1014808 | 2293523:21361206 | gi\|2293523\|gb\|AAB65422.1\|integrin variant beta4E [Homo sapiens] |
| HG1014809 | 239158:239157 | gi\|239158\|gb\|AAB20354.1\|integrin alpha 6A [Homo sapiens] |
| HG1014810 | 2432002:2432001 | gi\|2432002\|gb\|AAB71189.1\|Jagged 2 [Homo sapiens] |
| HG1014811 | 24496473:24496472 | gi\|24496473\|gb\|AAN60219.1\|peripherial benzodiazepine receptor-associated protein [Homo |
| | | sapiens] |
| HG1014812 | 24658543:24658542 | Similar to huntingtin interacting protein 1 related [Homo sapiens] |
| HG1014813 | 24659964:24659963 | gi\|24659964\|gb\|AAH39498.1\|SLC39A6 protein [Homo sapiens] |
| HG1014814 | 2598968:2598967 | gi\|2598968\|gb\|AAB84031.1\|Kunitz-type protease inhibitor [Homo sapiens] |
| HG1014815 | 2605947:2605946 | gi\|2605947\|gb\|AAB84216.1\|hJAG2.del-E6 [Homo sapiens] |
| HG1014816 | 2662364:2687860 | gi\|2662364\|dbj\|BAA23666.1\|DCRR1 [Homo sapiens] |
| HG1014817 | 2662375:473936 | gi\|2662375\|dbj\|BAA23670.1\|oligosaccharyltransferase [Homo sapiens] |
| HG1014818 | 27477822:27477821 | similar to Sel-1 homolog precursor (Suppressor of lin-12-like protein) (Sel-1L) [Homo sapiens] |
| HG1014819 | 27480564:27480563 | hypothetical protein XP_211921 [Homo sapiens] |
| HG1014820 | 27499509:27499508 | similar to Huntingtin interacting protein 1 related (Hip1-related) (Hip 12) [Homo sapiens] |
| HG1014821 | 27529860:27529859 | gi\|27529860\|dbj\|BAA86462.2\|KIAA1148 protein [Homo sapiens] |
| HG1014822 | 2765402:2765401 | gi\|2765402\|emb\|CAA74706.1\|jagged2 protein [Homo sapiens] |
| HG1014823 | 27694125:27694124 | gi\|27694125\|gb\|AAH43358.1\|Unknown (protein for IMAGE: 3904894) [Homo sapiens] |
| HG1014824 | 28175817:28175816 | gi\|28175817\|gb\|AAH43602.1\|PSME4 protein [Homo sapiens] |
| HG1014825 | 28207917:28207916 | gi\|28207917\|emb\|CAD62612.1\|unnamed protein product [Homo sapiens] |
| HG1014826 | 28273134:28273133 | gi\|28273134\|dbj\|BAC56930.1\|FLJ00414 protein [Homo sapiens] |
| HG1014827 | 28273138:28273137 | gi\|28273138\|dbj\|BAC56932.1\|FLJ00417 protein [Homo sapiens] |
| HG1014828 | 28277412:28277411 | gi\|28277412\|gb\|AAH44255.1\|NUP205 protein [Homo sapiens] |
| HG1014829 | 28279793:28279792 | gi\|28279793\|gb\|AAH46126.1\|ABCC3 protein [Homo sapiens] |
| HG1014830 | 28374245:28374244 | gi\|28374245\|gb\|AAH45549.1\|Carboxypeptidase D precursor [Homo sapiens] |
| HG1014831 | 285917:285916 | gi\|285917\|dbj\|BAA03537.1\|large erk kinase [Homo sapiens] |
| HG1014832 | 28981412:28981411 | gi\|28981412\|gb\|AAH48768.1\|PTPRF protein [Homo sapiens] |
| HG1014833 | 2924620:2924619 | gi\|2924620\|dbj\|BAA25024.1\|hepatocyte growth factor activator inhibitor type 2 [Homo sapiens] |
| HG1014834 | 2951948:7637876 | gi\|2951948\|gb\|AAC05440.1\|Unknown gene product [Homo sapiens] |
| HG1014835 | 30016:30015 | gi\|30016\|emb\|CAA30731.1\|unnamed protein product [Homo sapiens] |
| HG1014836 | 31223:31222 | gi\|31223\|emb\|CAA41981.1\|elk-related kinase [Homo sapiens] |
| HG1014837 | 3132270:3132269 | gi\|3132270\|dbj\|BAA28146.1\|multidrug resistance-associated protein(MRP)-like protein-2 |
| | | (MLP-2) [Homo sapiens] |
| HG1014838 | 3172147:219494 | gi\|3172147\|gb\|AAC18433.1\|BGP_HUMAN [Homo sapiens] |
| HG1014839 | 33911:33910 | gi\|33911\|emb\|CAA36134.1\|unnamed protein product [Homo sapiens] |
| HG1014840 | 33942:33941 | gi\|33942\|emb\|CAA42099.1\|integrin alpha6 subunit [Homo sapiens] |
| HG1014841 | 33957:33956 | gi\|33957\|emb\|CAA36433.1\|integrin beta 4 subunit [Homo sapiens] |
| HG1014842 | 35658:35657 | gi\|35658\|emb\|CAA25394.1\|prepro-alpha-1 collagen [Homo sapiens] |
| HG1014843 | 3582767:3582766 | gi\|3582767\|gb\|AAC35281.1\|putative erythrocyte intermediate conductance calcium-activated |
| | | potassium Gardos channel [Homo sapiens] |
| HG1014844 | 37200:37199 | gi\|37200\|emb\|CAA32940.1\|TM2-CEA precursor [Homo sapiens] |
| HG1014845 | 37204:37203 | gi\|37204\|emb\|CAA34405.1\|TM3-CEA protein [Homo sapiens] |
| HG1014846 | 3721836:3721835 | gi\|3721836\|dbj\|BAA33713.1\|HIP1R [Homo sapiens] |
| HG1014847 | 3721898:12804512 | gi\|3721898\|dbj\|BAA33736.1\|hJTB [Homo sapiens] |
| HG1014848 | 407590:407589 | gi\|407590\|gb\|AAB27856.1\|type I collagen pro alpha 1(I) chain propeptide [Homo sapiens] |
| HG1014849 | 4102188:4102187 | gi\|4102188\|gb\|AAD01430.1\|MRP3 [Homo sapiens] |
| HG1014850 | 4587083:4587082 | gi\|4587083\|dbj\|BAA76608.1\|MRP5 [Homo sapiens] |
| HG1014851 | 4755085:14719826 | gi\|4755085\|gb\|AAB94054.2\|pro alpha 1(I) collagen [Homo sapiens] |
| HG1014852 | 4826563:4826562 | gi\|4826563\|emb\|CAA76658.2\|multidrug resistance protein 3 (ABCC3) [Homo sapiens] |
| HG1014853 | 4836765:4836764 | gi\|4836765\|gb\|AAD30545.1\|G-protein-coupled receptor [Homo sapiens] |
| HG1014854 | 4894209:4894208 | gi\|4894209\|gb\|AAD32301.1\|cornichon-like protein [Homo sapiens] |
| HG1014855 | 495678:495677 | gi\|495678\|dbj\|BAA06506.1\|tyrosine kinase precursor [Homo sapiens] |
| HG1014856 | 5002294:4826835 | gi\|5002294\|gb\|AAD37404.1\|matrix metalloproteinase 9; MMP9; gelatinase B; type IV |
| | | collagenase [Homo sapiens] |
| HG1014857 | 5006891:5006890 | gi\|5006891\|gb\|AAD37716.1\|ABC protein [Homo sapiens] |
| HG1014858 | 5031476:5031475 | gi\|5031476\|gb\|AAD38185.1\|MRP3s1 protein [Homo sapiens] |
| HG1014859 | 5114047:5114046 | gi\|5114047\|gb\|AAD40191.1\|putative RNA helicase [Homo sapiens] |
| HG1014860 | 5726563:4557674 | gi\|5726563\|gb\|AAD48469.1\|integrin alpha 6 [Homo sapiens] |
| HG1014861 | 5851985:15488900 | gi\|5851985\|emb\|CAB55434.1\|dJ25J6.4 (ret finger protein) [Homo sapiens] |
| HG1014862 | 606777:29447 | gi\|606777\|emb\|CAA47694.1\|biliary glycoprotein [Homo sapiens] |
| HG1014863 | 6941892:6941891 | gi\|6941892\|gb\|AAF32265.1\|RFP transforming protein [Homo sapiens] |
| HG1014864 | 7022121:7022120 | gi\|7022121\|dbj\|BAA91495.1\|unnamed protein product [Homo sapiens] |
| HG1014865 | 7106834:7106833 | gi\|7106834\|gb\|AAF36142.1\|HSPC222 [Homo sapiens] |
| HG1014866 | 7159057:7159056 | gi\|7159057\|gb\|AAF37612.1\|type II iodothyronine deiodinase [Homo sapiens] |
| HG1014867 | 762938:30092 | gi\|762938\|emb\|CAA29605.1\|unnamed protein product [Homo sapiens] |
| HG1014868 | 7768766:4826652 | gi\|7768766\|dbj\|BAA95548.1\|C21orf5 [Homo sapiens] |
| HG1014869 | 7770185:7770184 | gi\|7770185\|gb\|AAF69628.1\|PRO2281 [Homo sapiens] |
| HG1014870 | proteinkinase320A:proteinkinase320B | gi\|38327632\|ref\|NP_001945.3\|discoidin receptor tyrosine kinase isoform b; PTK3A protein |
| | | tyrosine kinase 3A; cell adhesion kinase; epithelial discoidin domain receptor 1; neurotrophice |
| | | tyrosin kinase, receptor, type 4; neuroepithelial tyrosine kinase; mammarian carcinoma kinase |
| | | 10 [Homo sapiens] |
| HG1014871 | 307091:186775 | gi\|120749\|sp\|P16422\|TTD1_HUMAN Tumor-associated calcium signal transducer 1 precursor |
| | | (Major gastrointestinal tumor-associated protein GA733-2) (Epithelial cell surface antigen) |
| | | (Epithelial glycoprotein) (EGP) (Adenocarcinoma-associated antigen) (KSA) (KS 1/4 antigen) |
| | | (Cell surface glycoprotein Trop-1) |
| HG1014872 | 31417919:12803236 | gi\|31417919\|gb\|AAH02431.2\|B4GALT2 protein [Homo sapiens] |
| HG1014873 | 1160925:1160924 | gi\|38327632\|ref\|NP_001945.3\|discoidin receptor tyrosine kinase isoform b; PTK3A protein |
| | | tyrosine kinase 3A; cell adhesion kinase; epithelial discoidin domain receptor 1; neurotrophic |
| | | tyrosine kinase, receptor, type 4; neuroepithelial tyrosine kinase; mammarian.carcinoma kinase |
| | | 10 [Homo sapiens] |
| HG1014874 | 179435:179434 | gi\|86965\|pir\|\|JH0395 biliary glycoprotein h precursor - human |
| HG1014875 | 219497:219496 | gi\|86964\|pir\|\|JH0394 biliary glycoprotein g precursor - human |
| HG1014876 | 2554610:2554609 | gi\|7428837\|pir\|\|JC5667 multidrug resistance protein, short type - human |
| HG1014877 | 29387396:29387395 | gi\|29387396\|gb\|AAH48416.1\|PTPRF protein [Homo sapiens] |
| HG1014878 | 29421204:29421203 | gi\|29421204\|dbj\|BAB13462.2\|KIAA1636 protein [Homo sapiens] |
| HG1014879 | 29476766:29476765 | gi\|29476766\|gb\|AAH50037.1\|KIAA0450 protein [Homo sapiens] |
| HG1014880 | 29792320:29792319 | gi\|29792320\|gb\|AAH50744.1\|Unknown (protein for IMAGE: 6091533) [Homo sapiens] |
| HG1014881 | 30046456:30046455 | gi\|30046456\|gb\|AAH50370.1\|ABCC3 protein [Homo sapiens] |
| HG1014882 | 30046796:30046795 | gi\|30046796\|gb\|AAH50585.1\|ITGA6 protein [Homo sapiens] |
| HG1014883 | 30313820:30313819 | gi\|30313820\|gb\|AAO49801.1\|ATP-binding cassette C5 splicing variant A [Homo sapiens] |
| HG1014884 | 31323051:31323050 | gi\|31323051\|gb\|AAP44001.1\|hepatocyte growth factor activator inhibitor 1B [Homo sapiens] |
| HG1014885 | 31873230:31873229 | gi\|31873230\|emb\|CAD97607.1\|hypothetical protein [Homo sapiens] |
| HG1014886 | 32812254:32812253 | gi\|33186910\|ref\|NP_874365.1\|scribble isoform N1 [Homo sapiens] |
| HG1014887 | 32966069:32966068 | gi\|32966069\|gb\|AAP92131.1\|CD39L2 nucleotidase [Homo sapiens] |
| HG1014888 | 5825553:5825552 | gi\|12643871\|sp\|Q9UBM1\|PEMT_HUMAN Phosphatidylethanolamine N-methyltransferase |
| | | (PEAMT) (PEMT) (PEMT2) |
| HG1014889 | 11282038:6808452 | gi\|11282038\|pir\|\|T46511 hypothetical protein DKFZp586M2424.1 - human (fragment) |
| HG1014890 | 20138797:2605944 | gi\|20138797\|sp\|Q9Y219\|JAG2_HUMAN Jagged 2 precursor (Jagged2) (HJ2) |
| HG1014891 | 2136054:1060894 | gi\|2136054\|pir\|\|A57174 protein-tyrosine kinase (EC 2.7.1.112) erk - human (fragment) |
| HG1014892 | 2168139:6013007 | gi\|2168139\|emb\|CAB09423.1\|dJ105D12.1 (novel protein) [Homo sapiens] |
| HG1014893 | 25089854:3641620 | gi\|25089854\|sp\|O75976\|CBPD_HUMAN Carboxypeptidase D precursor (gp180) |
| HG1014894 | 263064:33941 | gi\|263064\|gb\|AAB24829.1\|integrin subunit alpha 6 [Homo sapiens] |
| HG1014895 | 32425685:12655128 | Unknown (protein for IMAGE: 3140321) [Homo sapiens] |
| HG1014896 | 7442652:3550323 | gi\|7442652\|pir\|\|JE0336 canalicular multispecific organic anion transporter - human |
| HG1014897 | 7459693:2293520 | gi\|7459693\|pir\|\|JC5545 integrin beta-4 precursor, splice form E - human |
| HG1014898 | 86966:219500 | gi\|86966\|pir\|\|JH0396 biliary glycoprotein i precursor - human |
| HG1014899 | 8928547:5685863 | gi\|8928547\|sp\|O15440\|MRP5_HUMAN Multidrug resistance-associated protein 5 |
| | | (Multi-specific organic anion tranporter-C) (MOAT-C) (pABC11) (SMRP) |
| HG1014900 | NP_857593.1:NM_181642 | gi\|32313599\|ref\|NP_857593.1\|hepatocyte growth factor activator inhibitor 1 isoform 1 |
| | | precursor; hepatocyte growth factor activator inhibitor 1; Kunitz-type protease inhibitor 1 |
| | | [Homo sapiens] |

TABLE 3
Protein Characteristics

| | | | | | Alternate | |
|---|---|---|---|---|---|---|
| | | Predicted | | Mature | Mature | Signal |
| | | Protein | Tree | Protein | Protein | Peptide |
| FP ID | Classification | Length | Vote | Coords | Coords | Coords |
| HG1014563 | STM TypeI_membrane | 456 | 0 | (1-456) | (45-456) | (17-44) |
| HG1014564 | KINASE STM | 1055 | 0.01 | (19-1055) | (22-1055) | (1-21) |
| HG1014566 | MTM | 267 | 0 | (1-267) | | |
| HG1014568 | PDE | 393 | 0.96 | (32-393) | (33-393) | (1-32) |
| HG1014569 | TypeI_membrane STM | 464 | 0 | (35-464) | (36-464) | (1-35) |
| HG1014570 | MTM | 580 | 0.02 | (1-580) | | |
| HG1014572 | TypeI_membrane STM | 526 | 0 | (35-526) | (36-526) | (1-35) |
| HG1014573 | MTM PHOSPHATASE | 288 | 0.19 | (20-288) | (25-288) | (1-24) |
| HG1014574 | KINASE STM | 509 | 0 | (18-509) | (1-509) | |
| HG1014575 | | 732 | 0.04 | (1-732) | | |
| HG1014578 | STM TypeI_membrane | 1117 | 0.01 | (23-1117) | | (1-22) |
| HG1014579 | SECRETED | 470 | 1 | (17-470) | (19-470) | (1-18) |
| | PROTEASE | | | | | |
| HG1014580 | MTM | 336 | 0.14 | (38-336) | | (17-37) |
| HG1014581 | STM | 1029 | 0 | (1-1029) | | |
| HG1014582 | MTM | 485 | 0.01 | (1-485) | | |
| HG1014583 | STM | 118 | 0 | (1-118) | | |
| | TypeIV_membrane | | | | | |
| HG1014584 | MTM | 438 | 0 | (1-438) | | |
| HG1014585 | | 134 | 0.32 | (1-134) | (37-134) | (8-36) |
| HG1014586 | MTM | 370 | 0.22 | (1-370) | (32-370) | (1-31) |
| HG1014587 | STM | 768 | 0 | (1-768) | | |
| HG1014589 | STM | 1805 | 0 | (1-1805) | | |
| HG1014590 | STM TypeI_membrane | 1871 | 0.99 | (1-1871) | (18-1871) | (1-17) |
| HG1014591 | PHOSPHATASE STM | 1897 | 0.01 | (20-1897) | | (1-19) |
| | TypeI_membrane | | | | | |
| HG1014593 | STM | 1214 | 0 | (36-1214) | (40-1214) | (16-39) |
| HG1014595 | MTM | 510 | 0 | (1-510) | | |
| HG1014597 | | 913 | 0 | (21-913) | | (1-20) |
| HG1014600 | MTM | 383 | 0.06 | (19-383) | (17-383) | (2-16) |
| HG1014601 | INTRACELLULAR | 2025 | 0.01 | (1-2025) | | |
| | UB_ligase | | | | | |
| HG1014602 | SECRETED | 1254 | 0.86 | (23-1254) | (28-1254) | (1-27) |
| HG1014603 | TypeI_membrane STM | 1380 | 0 | (30-1380) | (33-1380) | (1-32) |
| | PROTEASE | | | | | |
| HG1014604 | | 368 | 0.11 | (1-368) | (40-368) | (15-39) |
| HG1014605 | | 760 | 0.92 | (29-760) | (19-760) | (1-18) |
| HG1014606 | MTM | 532 | 0.04 | (35-532) | (1-532) | |
| HG1014607 | KINASE STM | 913 | 0 | (21-913) | | (1-20) |
| HG1014608 | MTM | 209 | 0.33 | (27-209) | (25-209) | (9-24) |
| HG1014609 | MTM | 217 | 0.47 | (29-217) | (26-217) | (11-25) |
| HG1014610 | STM TypeII_membrane | 836 | 0 | (1-836) | | |
| HG1014611 | PHOSPHATASE | 484 | 0 | (1-484) | | |
| | TypeII_membrane STM | | | | | |
| HG1014612 | STM TypeI_membrane | 201 | 0 | (21-201) | (20-201) | (1-19) |
| HG1014613 | INTRACELLULAR | 448 | 0.01 | (1-448) | | |
| HG1014614 | MTM | 199 | 0 | (1-199) | | |
| HG1014615 | KINASE STM | 998 | 0 | (38-998) | (36-998) | (1-35) |
| HG1014616 | MTM | 625 | 0.01 | (1-625) | | |
| HG1014617 | STM | 280 | 0.02 | (1-280) | (28-280) | (8-27) |
| HG1014618 | | 758 | 0.03 | (1-758) | | |
| HG1014619 | MTM | 1437 | 0.01 | (1-1437) | | |
| HG1014620 | SECRETED | 707 | 0.96 | (20-707) | | (1-19) |
| | PROTEASE | | | | | |
| HG1014621 | | 541 | 0.01 | (1-541) | | |
| HG1014622 | STM TypeI_membrane | 981 | 0 | (29-981) | (26-981) | (1-25) |
| HG1014623 | TypeI_membrane STM | 252 | 0 | (28-252) | | (1-27) |
| HG1014624 | STM | 1073 | 0 | (19-1073) | (23-1073) | (1-22) |
| HG1014625 | STM | 420 | 0.02 | (32-420) | | (17-31) |
| HG1014626 | STM TypeI_membrane | 1822 | 0.98 | (24-1822) | | (1-23) |
| HG1014627 | MTM | 144 | 0.41 | (21-144) | (19-144) | (1-18) |
| HG1014628 | STM TypeII_membrane | 355 | 0.03 | (34-355) | (1-355) | |
| HG1014629 | MTM | 506 | 0.16 | (19-506) | (23-506) | (1-22) |
| HG1014630 | STM | 165 | 0.01 | (1-165) | | |
| HG1014631 | SECRETED | 513 | 0 | (36-513) | | (6-35) |
| HG1014632 | TypeI_membrane STM | 314 | 0 | (24-314) | (19-314) | (1-18) |
| HG1014633 | MTM | 749 | 0.13 | (21-749) | (20-749) | (1-19) |
| HG1014634 | MTM | 427 | 0.01 | (37-427) | (1-427) | |
| HG1014635 | MTM | 511 | 0.04 | (1-511) | | |
| HG1014636 | MTM | 220 | 0 | (24-220) | (1-220) | |
| HG1014637 | STM TypeII_membrane | 373 | 0.99 | (28-373) | | (9-27) |
| HG1014638 | INTRACELLULAR | 1630 | 0 | (1-1630) | | |
| HG1014639 | STM TypeI_membrane | 616 | 0 | (25-616) | (27-616) | (1-26) |
| HG1014640 | PROTEASE STM | 824 | 0 | (20-824) | (19-824) | (1-18) |
| | TypeI_membrane | | | | | |
| HG1014641 | PROTEASE STM | 775 | 0 | (18-775) | | (1-17) |
| | TypeI_membrane | | | | | |
| HG1014642 | STM TypeII_membrane | 478 | 0 | (27-478) | (23-478) | (1-22) |
| HG1014643 | STM TypeI_membrane | 146 | 0 | (31-146) | (33-146) | (1-32) |
| HG1014644 | MTM | 687 | 0.07 | (26-687) | (27-687) | (1-26) |
| HG1014645 | STM TypeI_membrane | 1238 | 0.01 | (27-1238) | | (4-26) |
| HG1014646 | STM TypeII_membrane | 344 | 0.98 | (28-344) | (32-344) | (1-31) |
| HG1014647 | INTRACELLULAR | 2013 | 0 | (1-2013) | | |
| HG1014692 | PROTEASE STM | 540 | 1 | (18-540) | | (1-17) |
| | TypeI_membrane | | | | | |
| HG1014693 | PROTEASE STM | 775 | 0 | (18-775) | | (1-17) |
| | TypeI_membrane | | | | | |
| HG1014694 | MTM | 393 | 0.02 | (1-393) | | |
| HG1014695 | MTM | 361 | 0 | (1-361) | | |
| HG1014696 | MTM | 393 | 0.02 | (1-393) | | |
| HG1014697 | MTM | 526 | 0 | (29-526) | (1-526) | |
| HG1014698 | MTM | 147 | 0.02 | (1-147) | | |
| HG1014699 | INTRACELLULAR | 279 | 0 | (24-279) | (1-279) | |
| HG1014700 | STM TypeII_membrane | 136 | 0.98 | (28-136) | | (9-27) |
| HG1014701 | MTM | 355 | 0 | (1-355) | | |
| HG1014702 | INTRACELLULAR | 374 | 0.27 | (32-374) | (34-374) | (1-33) |
| HG1014703 | INTRACELLULAR | 358 | 0 | (1-358) | (48-358) | (19-47) |
| | UB_ligase | | | | | |
| HG1014704 | MTM | 528 | 0.05 | (1-528) | | |
| HG1014705 | INTRACELLULAR | 1101 | 0.03 | (1-1101) | | |
| HG1014706 | INTRACELLULAR | 2863 | 0 | (1-2863) | | |
| HG1014707 | KINASE STM | 998 | 0 | (38-998) | (36-998) | (1-35) |
| | TypeI_membrane | | | | | |
| | pkinase_EphB3 | | | | | |
| HG1014708 | INTRACELLULAR | 1630 | 0 | (1-1630) | | |
| HG1014709 | INTRACELLULAR | 1806 | 0.21 | (36-1806) | (35-1806) | (1-34) |
| HG1014710 | PHOSPHATASE STM | 1888 | 0.01 | (20-1888) | | (1-19) |
| | TypeI_membrane | | | | | |
| HG1014711 | MTM | 693 | 0.07 | (26-693) | (27-693) | (1-26) |
| HG1014712 | STM TypeI_membrane | 456 | 0 | (1-456) | (45-456) | (17-44) |
| HG1014713 | KINASE STM | 987 | 0.01 | (19-987) | (22-987) | (1-21) |
| | TypeI_membrane | | | | | |
| | pkinase_EphB2 | | | | | |
| HG1014714 | STM TypeI_membrane | 1200 | 0.01 | (27-1200) | | (4-26) |
| HG1014715 | TypeI_membrane STM | 1380 | 0 | (30-1380) | (33-1380) | (1-32) |
| | PROTEASE | | | | | |
| HG1014716 | MTM | 236 | 0.01 | (1-236) | | |
| HG1014717 | MTM | 199 | 0 | (1-199) | | |
| HG1014718 | INTRACELLULAR | 1811 | 0.53 | (15-1811) | | (1-14) |
| HG1014719 | MTM PHOSPHATASE | 232 | 0 | (16-232) | (1-232) | |
| HG1014720 | MTM PHOSPHATASE | 309 | 0.01 | (1-309) | | |
| HG1014721 | STM TypeII_membrane | 372 | 0.99 | (28-372) | | (9-27) |
| HG1014722 | STM TypeII_membrane | 1464 | 0.99 | (23-1464) | | (1-22) |
| HG1014723 | STM | 1215 | 0 | (20-1215) | | (1-19) |
| HG1014724 | PHOSPHATASE | 484 | 0 | (1-484) | | |
| | TypeII_membrane STM | | | | | |
| HG1014725 | MTM | 506 | 0.16 | (19-506) | (23-506) | (1-22) |
| HG1014726 | INTRACELLULAR | 1128 | 0.03 | (1-1128) | | |
| HG1014727 | STM | 273 | 0.7 | (1-273) | (31-273) | (15-30) |
| HG1014728 | KINASE STM | 913 | 0 | (21-913) | | (1-20) |
| | TypeI_membrane | | | | | |
| | pkinase_DDR1 | | | | | |
| HG1014729 | KINASE STM | 919 | 0 | (21-919) | | (1-20) |
| | TypeI_membrane | | | | | |
| | pkinase_DDR1 | | | | | |
| HG1014730 | SECRETED | 1405 | 0.01 | (1-1405) | | |
| HG1014731 | PDE | 331 | 0.96 | (32-331) | (33-331) | (1-32) |
| HG1014732 | MTM | 842 | 0 | (1-842) | | |
| HG1014733 | MTM | 1527 | 0.01 | (1-1527) | | |
| HG1014734 | MTM | 1238 | 0.01 | (1-1238) | | |
| HG1014735 | INTRACELLULAR | 947 | 0 | (1-947) | | |
| HG1014736 | INTRACELLULAR | 774 | 0.52 | (15-774) | | (1-14) |
| HG1014737 | MTM | 528 | 0.05 | (1-528) | | |
| HG1014738 | SECRETED | 707 | 0.96 | (20-707) | | (1-19) |
| | PROTEASE | | | | | |
| HG1014739 | STM | 309 | 0.71 | (1-309) | (31-309) | (15-30) |
| HG1014740 | STM | 93 | 0.1 | (1-93) | (19-93) | (1-18) |
| HG1014741 | MTM | 896 | 0.02 | (1-896) | | |
| HG1014742 | MTM | 283 | 0 | (1-283) | | |
| HG1014743 | INTRACELLULAR | 337 | 0.01 | (1-337) | | |
| HG1014744 | STM TypeII_membrane | 344 | 0.98 | (28-344) | (32-344) | (1-31) |
| HG1014745 | KINASE STM | 552 | 0 | (1-552) | | |
| | TypeI_membrane | | | | | |
| | pkinase_EphB2 | | | | | |
| HG1014746 | KINASE STM | 621 | 0 | (1-621) | | |
| | TypeI_membrane | | | | | |
| | pkinase_EphB2 | | | | | |
| HG1014747 | MTM | 236 | 0.01 | (1-236) | | |
| HG1014748 | STM | 74 | 0.58 | (23-74) | (31-74) | (15-30) |
| HG1014749 | INTRACELLULAR | 59 | 0.04 | (1-59) | (25-59) | (1-24) |
| HG1014750 | STM TypeII_membrane | 562 | 0 | (1-562) | | |
| HG1014751 | MTM | 433 | 0 | (1-433) | | |
| HG1014752 | STM TypeII_membrane | 344 | 0.98 | (28-344) | (32-344) | (1-31) |
| HG1014753 | SECRETED | 186 | 0.07 | (1-186) | (17-186) | (3-16) |
| HG1014754 | SECRETED | 186 | 0.07 | (1-186) | (17-186) | (3-16) |
| HG1014755 | KINASE STM | 82 | 0.92 | (9-82) | (19-82) | (4-18) |
| | TypeI_membrane | | | | | |
| | pkinase_DDR1 | | | | | |
| HG1014756 | KINASE STM | 62 | 0.91 | (9-62) | (19-62) | (4-18) |
| | TypeI_membrane | | | | | |
| | pkinase_DDR1 | | | | | |
| HG1014757 | MTM | 39 | 0.26 | (8-39) | (29-39) | (11-28) |
| HG1014758 | INTRACELLULAR | 1308 | 0.04 | (1-1308) | | |
| HG1014759 | TypeI_membrane STM | 252 | 0.01 | (28-252) | | (1-27) |
| HG1014760 | MTM | 382 | 0 | (11-382) | (1-382) | |
| HG1014761 | PROTEASE | 714 | 0 | (17-714) | (1-714) | |
| | INTRACELLULAR | | | | | |
| HG1014762 | INTRACELLULAR | 453 | 0 | (1-453) | | |
| HG1014763 | STM | 705 | 0 | (1-705) | | |
| HG1014764 | INTRACELLULAR | 1729 | 0 | (1-1729) | | |
| HG1014765 | KINASE STM | 108 | 0.15 | (24-108) | (27-108) | (11-26) |
| | TypeI_membrane | | | | | |
| | pkinase_EphB2 | | | | | |
| HG1014766 | MTM | 427 | 0.01 | (37-427) | (1-427) | |
| HG1014767 | INTRACELLULAR | 24 | 0.01 | (1-24) | (18-24) | (1-17) |
| HG1014768 | STM | 83 | 0 | (1-83) | (17-83) | (2-16) |
| | TypeIV_membrane | | | | | |
| HG1014769 | MTM | 1514 | 0 | (1-1514) | | |
| HG1014770 | TypeI_membrane STM | 468 | 0 | (35-468) | (36-468) | (1-35) |
| HG1014771 | INTRACELLULAR | 835 | 0.01 | (1-835) | | |
| HG1014772 | MTM | 49 | 0 | (1-49) | | |
| HG1014773 | INTRACELLULAR | 1715 | 0 | (1-1715) | | |
| | UB_ligase | | | | | |
| HG1014774 | MTM | 766 | 0 | (1-766) | | |
| HG1014775 | SECRETED | 240 | 0 | (1-240) | | |
| | PROTEASE | | | | | |
| HG1014776 | MTM | 505 | 0.16 | (19-505) | (23-505) | (1-22) |
| HG1014777 | STM TypeII_membrane | 66 | 0 | (1-66) | | |
| HG1014778 | STM TypeII_membrane | 41 | 0.02 | (1-41) | (34-41) | (2-33) |
| HG1014779 | STM TypeII_membrane | 60 | 0 | (1-60) | | |
| HG1014780 | INTRACELLULAR | 117 | 0 | (1-117) | | |
| HG1014781 | INTRACELLULAR | 716 | 0 | (1-716) | | |
| HG1014782 | INTRACELLULAR | 656 | 0.03 | (1-656) | | |
| HG1014783 | STM TypeII_membrane | 1069 | 1 | (23-1069) | | (1-22) |
| HG1014784 | PHOSPHATASE | 483 | 0 | (1-483) | | |
| | TypeII_membrane STM | | | | | |
| HG1014785 | STM TypeII_membrane | 837 | 0 | (1-837) | | |
| HG1014786 | INTRACELLULAR | 2147 | 0.02 | (27-2147) | (1-2147) | |
| HG1014787 | INTRACELLULAR | 1844 | 0.01 | (1-1844) | | |
| HG1014788 | MTM | 552 | 0.02 | (1-552) | | |
| HG1014789 | STM TypeI_membrane | 439 | 0 | (26-439) | (27-439) | (1-26) |
| HG1014790 | INTRACELLULAR | 2851 | 0 | (1-2851) | | |
| HG1014791 | STM TypeI_membrane | 971 | 0 | (29-971) | (26-971) | (1-25) |
| HG1014792 | PHOSPHATASE | 503 | 0 | (1-503) | | |
| | TypeII_membrane STM | | | | | |
| HG1014793 | INTRACELLULAR | 625 | 0 | (1-625) | | |
| HG1014794 | INTRACELLULAR | 399 | 0.06 | (1-399) | (37-399) | (14-36) |
| HG1014795 | INTRACELLULAR | 391 | 0.02 | (1-391) | | |
| HG1014796 | STM | 657 | 0 | (1-657) | | |
| HG1014797 | MTM | 693 | 0.07 | (26-693) | (27-693) | (1-26) |
| HG1014798 | TypeI_membrane STM | 461 | 0 | (35-461) | (36-461) | (1-35) |
| HG1014799 | MTM | 364 | 0 | (1-364) | | |
| HG1014800 | STM TypeI_membrane | 1238 | 0.01 | (27-1238) | | (4-26) |
| HG1014801 | STM | 629 | 0 | (37-629) | (36-629) | (1-35) |
| HG1014802 | STM TypeII_membrane | 1464 | 0.99 | (23-1464) | | (1-22) |
| HG1014803 | SECRETED | 707 | 0.96 | (20-707) | | (1-19) |
| | PROTEASE | | | | | |
| HG1014804 | STM TypeI_membrane | 1752 | 0.98 | (24-1752) | | (1-23) |
| HG1014805 | STM TypeI_membrane | 1822 | 0.98 | (24-1822) | | (1-23) |
| | TypeIV_membrane | | | | | |
| HG1014769 | MTM | 1514 | 0 | (1-1514) | | |
| HG1014770 | TypeI_membrane STM | 468 | 0 | (35-468) | (36-468) | (1-35) |
| HG1014771 | INTRACELLULAR | 835 | 0.01 | (1-835) | | |
| HG1014772 | MTM | 49 | 0 | (1-49) | | |
| HG1014773 | INTRACELLULAR | 1715 | 0 | (1-1715) | | |
| | UB_ligase | | | | | |
| HG1014774 | MTM | 766 | 0 | (1-766) | | |
| HG1014775 | SECRETED | 240 | 0 | (1-240) | | |
| | PROTEASE | | | | | |
| HG1014776 | MTM | 505 | 0.16 | (19-505) | (23-505) | (1-22) |
| HG1014777 | STM TypeII_membrane | 66 | 0 | (1-66) | | |
| HG1014778 | STM TypeII_membrane | 41 | 0.02 | (1-41) | (34-41) | (2-33) |
| HG1014779 | STM TypeII_membrane | 60 | 0 | (1-60) | | |
| HG1014780 | INTRACELLULAR | 117 | 0 | (1-117) | | |
| HG1014781 | INTRACELLULAR | 716 | 0 | (1-716) | | |
| HG1014782 | INTRACELLULAR | 656 | 0.03 | (1-656) | | |
| HG1014783 | STM TypeII_membrane | 1069 | 1 | (23-1069) | | (1-22) |
| HG1014784 | PHOSPHATASE | 483 | 0 | (1-483) | | |
| | TypeII_membrane STM | | | | | |
| HG1014785 | STM TypeII_membrane | 837 | 0 | (1-837) | | |
| HG1014786 | INTRACELLULAR | 2147 | 0.02 | (27-2147) | (1-2147) | |
| HG1014787 | INTRACELLULAR | 1844 | 0.01 | (1-1844) | | |
| HG1014788 | MTM | 552 | 0.02 | (1-552) | | |
| HG1014789 | STM TypeI_membrane | 439 | 0 | (26-439) | (27-439) | (1-26) |
| HG1014790 | INTRACELLULAR | 2851 | 0 | (1-2851) | | |
| HG1014791 | STM TypeI_membrane | 971 | 0 | (29-971) | (26-971) | (1-25) |
| HG1014792 | PHOSPHATASE | 503 | 0 | (1-503) | | |
| | TypeII_membrane STM | | | | | |
| HG1014793 | INTRACELLULAR | 625 | 0 | (1-625) | | |
| HG1014794 | INTRACELLULAR | 399 | 0.06 | (1-399) | (37-399) | (14-36) |
| HG1014795 | INTRACELLULAR | 391 | 0.02 | (1-391) | | |
| HG1014796 | STM | 657 | 0 | (1-657) | | |
| HG1014797 | MTM | 693 | 0.07 | (26-693) | (27-693) | (1-26) |
| HG1014798 | TypeI_membrane STM | 461 | 0 | (35-461) | (36-461) | (1-35) |
| HG1014799 | MTM | 364 | 0 | (1-364) | | |
| HG1014800 | STM TypeI_membrane | 1238 | 0.01 | (27-1238) | | (4-26) |
| HG1014801 | STM | 629 | 0 | (37-629) | (36-629) | (1-35) |
| HG1014802 | STM TypeII_membrane | 1464 | 0.99 | (23-1464) | | (1-22) |
| HG1014803 | SECRETED | 707 | 0.96 | (20-707) | | (1-19) |
| | PROTEASE | | | | | |
| HG1014804 | STM TypeI_membrane | 1752 | 0.98 | (24-1752) | | (1-23) |
| HG1014805 | STM TypeI_membrane | 1822 | 0.98 | (24-1822) | | (1-23) |
| HG1014806 | STM TypeI_membrane | 1805 | 0.98 | (24-1805) | | (1-23) |
| HG1014807 | MTM | 506 | 0.16 | (19-506) | (23-506) | (1-22) |
| HG1014808 | STM TypeI_membrane | 76 | 0.04 | (2-76) | (30-76) | (15-29) |
| HG1014809 | STM | 147 | 0.02 | (1-147) | | |
| HG1014810 | STM TypeI_membrane | 1238 | 0 | (24-1238) | (27-1238) | (1-26) |
| HG1014811 | MTM | 528 | 0.06 | (1-528) | | |
| HG1014812 | INTRACELLULAR | 501 | 0.98 | (23-501) | (24-501) | (1-23) |
| HG1014813 | MTM | 433 | 0 | (1-433) | | |
| HG1014814 | TypeI_membrane STM | 252 | 0.09 | (28-252) | | (1-27) |
| HG1014815 | STM TypeI_membrane | 1200 | 0.01 | (27-1200) | | (4-26) |
| HG1014816 | INTRACELLULAR | 1941 | 0 | (1-1941) | | |
| | UB_ligase | | | | | |
| HG1014817 | STM TypeI_membrane | 456 | 0 | (1-456) | (45-456) | (17-44) |
| HG1014818 | STM | 455 | 0 | (31-455) | (1-455) | |
| HG1014819 | STM | 100 | 0.67 | (20-100) | | (2-19) |
| HG1014820 | INTRACELLULAR | 1068 | 0 | (1-1068) | | |
| HG1014821 | INTRACELLULAR | 543 | 0 | (1-543) | | |
| HG1014822 | STM TypeI_membrane | 1223 | 0 | (27-1223) | (1-1223) | |
| HG1014823 | INTRACELLULAR | 1238 | 0 | (18-1238) | (1-1238) | |
| HG1014824 | INTRACELLULAR | 459 | 0.01 | (31-459) | (1-459) | |
| HG1014825 | MTM | 94 | 0.44 | (19-94) | (27-94) | (9-26) |
| HG1014826 | INTRACELLULAR | 1129 | 0 | (1-1129) | | |
| HG1014827 | INTRACELLULAR | 600 | 0 | (38-600) | (1-600) | |
| HG1014828 | INTRACELLULAR | 832 | 0 | (1-832) | | |
| HG1014829 | MTM | 598 | 0 | (1-598) | | |
| HG1014830 | TypeI_membrane STM | 1380 | 0 | (30-1380) | (33-1380) | (1-32) |
| | PROTEASE | | | | | |
| HG1014831 | KINASE STM | 347 | 0 | (1-347) | | |
| | TypeI_membrane | | | | | |
| | pkinase_EphB2 | | | | | |
| HG1014832 | PHOSPHATASE STM | 1898 | 0 | (30-1898) | | (11-29) |
| | TypeI_membrane | | | | | |
| HG1014833 | TypeI_membrane STM | 252 | 0.05 | (28-252) | (34-252) | (1-33) |
| HG1014834 | PDE | 119 | 0.04 | (21-119) | (1-119) | |
| HG1014835 | STM TypeII_membrane | 472 | 0.99 | (23-472) | | (1-22) |
| HG1014836 | KINASE STM | 61 | 0 | (1-61) | | |
| | TypeI_membrane | | | | | |
| | pkinase_EphB2 | | | | | |
| HG1014837 | MTM | 1527 | 0.01 | (1-1527) | | |
| HG1014838 | TypeI_membrane STM | 461 | 0 | (35-461) | (36-461) | (1-35) |
| HG1014839 | STM TypeI_membrane | 1752 | 0.98 | (24-1752) | | (1-23) |
| HG1014840 | STM | 1067 | 0 | (9-1067) | (17-1067) | (2-16) |
| HG1014841 | STM TypeI_membrane | 1805 | 0.98 | (24-1805) | | (1-23) |
| HG1014842 | STM TypeII_membrane | 181 | 0.99 | (1-181) | (23-181) | (1-22) |
| HG1014843 | MTM | 141 | 0 | (29-141) | (1-141) | |
| HG1014844 | TypeI_membrane STM | 430 | 0 | (35-430) | (36-430) | (1-35) |
| HG1014845 | TypeI_membrane STM | 373 | 0.01 | (1-373) | | |
| HG1014846 | INTRACELLULAR | 890 | 0.02 | (32-890) | (1-890) | |
| HG1014847 | STM TypeI_membrane | 94 | 0.85 | (31-94) | (33-94) | (1-32) |
| HG1014848 | STM TypeII_membrane | 284 | 0.05 | (1-284) | (31-284) | (1-30) |
| HG1014849 | MTM | 1528 | 0.01 | (1-1528) | | |
| HG1014850 | MTM | 1437 | 0.01 | (1-1437) | | |
| HG1014851 | STM TypeII_membrane | 1461 | 0.99 | (23-1461) | | (1-22) |
| HG1014852 | MTM | 1527 | 0.01 | (1-1527) | | |
| HG1014853 | MTM | 693 | 0.07 | (26-693) | (27-693) | (1-26) |
| HG1014854 | MTM | 134 | 0.31 | (11-134) | (19-134) | (1-18) |
| HG1014855 | KINASE STM | 981 | 0 | (15-981) | (16-981) | (1-15) |
| | TypeI_membrane | | | | | |
| | pkinase_EphB2 | | | | | |
| HG1014856 | SECRETED | 79 | 0.99 | (20-79) | | (1-19) |
| | PROTEASE | | | | | |
| HG1014857 | MTM | 1437 | 0.01 | (1-1437) | | |
| HG1014858 | MTM | 285 | 0 | (1-285) | | |
| HG1014859 | INTRACELLULAR | 595 | 0 | (1-595) | | |
| HG1014860 | STM | 1073 | 0 | (19-1073) | (23-1073) | (1-22) |
| HG1014861 | INTRACELLULAR | 249 | 0 | (1-249) | (48-249) | (19-47) |
| | UB_ligase | | | | | |
| HG1014862 | TypeI_membrane STM | 21 | 0.19 | (1-21) | (19-21) | (3-18) |
| HG1014863 | INTRACELLULAR | 47 | 0.07 | (1-47) | | (19-47) |
| | UB_ligase | | | | | |
| HG1014864 | INTRACELLULAR | 161 | 0.03 | (1-161) | | |
| HG1014865 | STM TypeI_membrane | 117 | 0.36 | (16-117) | (19-117) | (1-18) |
| HG1014866 | STM | 115 | 0.66 | (23-115) | (31-115) | (15-30) |
| HG1014867 | STM TypeII_membrane | 226 | 0 | (1-226) | | |
| HG1014868 | INTRACELLULAR | 2298 | 0.01 | (1-2298) | | |
| HG1014869 | INTRACELLULAR | 329 | 0 | (1-329) | | |
| HG1014870 | KINASE STM | 875 | 0 | (21-875) | | (1-20) |
| | TypeI_membrane | | | | | |
| | pkinase_DDR1 | | | | | |
| HG1014871 | TypeI_membrane STM | 314 | 0 | (24-314) | (19-314) | (1-18) |
| HG1014872 | STM TypeII_membrane | 306 | 0.43 | (16-306) | (19-396) | (1-18) |
| HG1014873 | KINASE STM | 876 | 0 | (21-876) | | (1-20) |
| | TypeI_membrane | | | | | |
| | pkinase_DDR1 | | | | | |
| HG1014874 | TypeI_membrane STM | 321 | 0.72 | (35-321) | (36-321) | (1-35) |
| HG1014875 | TypeI_membrane STM | 417 | 0.76 | (35-417) | (36-417) | (1-35) |
| HG1014876 | MTM | 946 | 0.01 | (1-946) | | |
| HG1014877 | PHOSPHATASE STM | 353 | 0.94 | (30-353) | | (11-29) |
| | TypeI_membrane | | | | | |
| HG1014878 | INTRACELLULAR | 1864 | 0.01 | (14-1864) | (1-1864) | |
| HG1014879 | INTRACELLULAR | 501 | 0.01 | (1-501) | | |
| HG1014880 | MTM | 208 | 0 | (1-208) | | |
| HG1014881 | MTM | 573 | 0 | (1-573) | | |
| HG1014882 | STM | 686 | 0 | (1-686) | | |
| HG1014883 | MTM | 1394 | 0.01 | (1-1394) | | |
| HG1014884 | SECRETED | 529 | 0 | (36-529) | | (6-35) |
| HG1014885 | PHOSPHATASE STM | 1191 | 0 | (1-1191) | | |
| | TypeI_membrane | | | | | |
| HG1014886 | INTRACELLULAR | 1601 | 0.18 | (1-1601) | (38-1601) | (6-37) |
| HG1014887 | PHOSPHATASE | 484 | 0 | (1-484) | | |
| | TypeII_membrane STM | | | | | |
| HG1014888 | MTM | 199 | 0 | (1-199) | | |
| HG1014889 | STM TypeII_membrane | 224 | 0 | (1-224) | | |
| HG1014890 | STM TypeI_membrane | 1238 | 0.01 | (27-1238) | | (4-26) |
| HG1014891 | KINASE STM | 478 | 0 | (1-478) | | |
| | TypeI_membrane | | | | | |
| | pkinase_EphB2 | | | | | |
| HG1014892 | SECRETED | 135 | 0.07 | (38-135) | (47-135) | (15-46) |
| HG1014893 | TypeI_membrane STM | 1380 | 0 | (30-1380) | (33-1380) | (1-32) |
| | PROTEASE | | | | | |
| HG1014894 | STM | 102 | 0.01 | (1-102) | | |
| HG1014895 | INTRACELLULAR | 497 | 0 | (1-497) | | |
| HG1014896 | MTM | 1527 | 0.01 | (1-1527) | | |
| HG1014897 | STM TypeI_membrane | 964 | 0 | (24-964) | | (1-23) |
| HG1014898 | TypeI_membrane STM | 351 | 0.76 | (35-351) | (36-351) | (1-35) |
| HG1014899 | MTM | 1437 | 0.01 | (1-1437) | | |
| HG1014900 | SECRETED | 529 | 0 | (36-529) | | (6-35) |

| FP ID | TM | TM Coords | non-TM Coords |
|---|---|---|---|
| HG1014563 | 2 | (21-43)(425-447) | (1-20)(44-424)(448-456) |
| HG1014564 | 1 | (543-565) | (1-542)(566-1055) |
| HG1014566 | 4 | (55-77)(133-155) | (1-54)(78-132)(156-188) |
| | | (189-211)(226-245) | (212-225)(246-267) |
| HG1014568 | 1 | (7-29) | (1-6)(30-393) |
| HG1014569 | 1 | (430-452) | (1-429)(453-464) |
| HG1014570 | 9 | (63-85)(119-136) | (1-62)(86-118)(137-140) |
| | | (141-163)(167-186) | (164-166)(187-206) |
| | | (207-229)(244-263) | (230-243)(264-314) |
| | | (315-337)(357-376) | (338-356)(377-396) |
| | | (397-419) | (420-580) |
| HG1014572 | 1 | (433-455) | (1-432)(456-526) |
| HG1014573 | 6 | (5-27)(56-78)(91-113) | (1-4)(28-55)(79-90)(114-162) |
| | | (163-185)(198-215) | (186-197)(216-224) |
| | | (225-247) | (248-288) |
| HG1014574 | 1 | (124-146) | (1-123)(147-509) |
| HG1014575 | 8 | (275-297)(312-334) | (1-274)(298-311)(335-346) |
| | | (347-369)(399-421) | (370-398)(422-487) |
| | | (488-505)(543-565) | (506-542)(566-618) |
| | | (619-641)(656-678) | (642-655)(679-732) |
| HG1014578 | 2 | (7-29)(612-634) | (1-6)(30-611)(635-1117) |
| HG1014579 | 0 | | (1-470) |
| HG1014580 | 5 | (20-42)(135-157) | (1-19)(43-134)(158-177) |
| | | (178-200)(210-232) | (201-209)(233-244) |
| | | (245-267) | (268-336) |
| HG1014581 | 1 | (950-972) | (1-949)(973-1029) |
| HG1014582 | 3 | (34-56)(61-83)(152-174) | (1-33)(57-60)(84-151) |
| | | | (175-485) |
| HG1014583 | 1 | (96-115) | (1-95)(116-118) |
| HG1014584 | 7 | (43-65)(96-115)(128-147) | (1-42)(66-95)(116-127) |
| | | (167-189)(201-223) | (148-166)(190-200) |
| | | (284-306)(360-382) | (224-283)(307-359) |
| | | | (383-438) |
| HG1014585 | 1 | (20-42) | (1-19)(43-134) |
| HG1014586 | 4 | (20-42)(54-76)(126-148) | (1-19)(43-53)(77-125) |
| | | (339-361) | (149-338)(362-370) |
| HG1014587 | 1 | (715-737) | (1-714)(738-768) |
| HG1014589 | 2 | (1005-1027)(1040-1062) | (1-1004)(1028-1039) |
| | | | (1063-1805) |
| HG1014590 | 0 | | (1-1871) |
| HG1014591 | 1 | (1252-1274) | (1-1251)(1275-1897) |
| HG1014593 | 1 | (1145-1167) | (1-1144)(1168-1214) |
| HG1014595 | 5 | (37-56)(63-85)(100-122) | (1-36)(57-62)(86-99) |
| | | (134-153)(168-190) | (123-133)(154-167) |
| | | | (191-510) |
| HG1014597 | 1 | (417-439) | (1-416)(440-913) |
| HG1014600 | 9 | (65-87)(99-118)(160-182) | (1-64)(88-98)(119-159) |
| | | (187-206)(216-233) | (183-186)(207-215) |
| | | (245-267)(282-304) | (234-244)(268-281) |
| | | (309-331)(336-358) | (305-308)(332-335) |
| | | | (359-383) |
| HG1014601 | 0 | | (1-2025) |
| HG1014602 | 1 | (7-25) | (1-6)(26-1254) |
| HG1014603 | 2 | (13-32)(1300-1322) | (1-12)(33-1299)(1323-1380) |
| HG1014604 | 0 | | (1-368) |
| HG1014605 | 1 | (7-29) | (1-6)(30-760) |
| HG1014606 | 10 | (7-29)(109-128)(149-171) | (1-6)(30-108)(129-148) |
| | | (198-220)(241-263) | (172-197)(221-240) |
| | | (338-360)(367-386) | (264-337)(361-366) |
| | | (433-455)(467-489) | (387-432)(456-466) |
| | | (499-521) | (490-498)(522-532) |
| HG1014607 | 1 | (417-439) | (1-416)(440-913) |
| HG1014608 | 4 | (12-34)(77-99)(119-141) | (1-11)(35-76)(100-118) |
| | | (161-183) | (142-160)(184-209) |
| HG1014609 | 4 | (5-27)(77-99)(123-145) | (1-4)(28-76)(100-122) |
| | | (160-182) | (146-159)(183-217) |
| HG1014610 | 1 | (42-64) | (1-41)(65-836) |
| HG1014611 | 1 | (38-60) | (1-37)(61-484) |
| HG1014612 | 2 | (4-23)(169-191) | (1-3)(24-168)(192-201) |
| HG1014613 | 0 | | (1-448) |
| HG1014614 | 4 | (13-35)(45-67)(88-110) | (1-12)(36-44)(68-87) |
| | | (157-179) | (111-156)(180-199) |
| HG1014615 | 1 | (560-582) | (1-559)(583-998) |
| HG1014616 | 9 | (263-285)(329-351) | (1-262)(286-328)(352-360) |
| | | (361-383)(396-418) | (384-395)(419-432) |
| | | (433-455)(483-505) | (456-482)(506-514) |
| | | (515-537)(550-572) | (538-549)(573-586) |
| | | (587-609) | (610-625) |
| HG1014617 | 0 | | (1-280) |
| HG1014618 | 5 | (123-145)(160-182) | (1-122)(146-159)(183-194) |
| | | (195-214)(249-271) | (215-248)(272-277) |
| | | (278-300) | (301-758) |
| HG1014619 | 11 | (179-201)(216-238) | (1-178)(202-215)(239-292) |
| | | (293-315)(320-342) | (316-319)(343-395) |
| | | (396-418)(428-447) | (419-427)(448-856) |
| | | (857-879)(914-936) | (880-913)(937-992) |
| | | (993-1010) | (1011-1013)(1037-1096) |
| | | (1014-1036) | (1120-1437) |
| | | (1097-1119) | |
| HG1014620 | 0 | | (1-707) |
| HG1014621 | 9 | (53-75)(95-117)(130-152) | (1-52)(76-94)(118-129) |
| | | (228-245)(265-287) | (153-227)(246-264) |
| | | (302-324)(337-359) | (288-301)(325-336) |
| | | (379-401)(414-436) | (360-378)(402-413) |
| | | | (437-541) |
| HG1014622 | 1 | (860-882) | (1-859)(883-981) |
| HG1014623 | 1 | (199-221) | (1-198)(222-252) |
| HG1014624 | 1 | (1015-1037) | (1-1014)(1038-1073) |
| HG1014625 | 1 | (354-376) | (1-353)(377-420) |
| HG1014626 | 0 | | (1-1822) |
| HG1014627 | 3 | (7-29)(56-78)(123-142) | (1-6)(30-55)(79-122) |
| | | | (143-144) |
| HG1014628 | 4 | (15-37)(76-98)(123-145) | (1-14)(38-75)(99-122) |
| | | (150-172) | (146-149)(173-355) |
| HG1014629 | 4 | (279-301)(308-327) | (1-278)(302-307)(328-341) |
| | | (342-364)(482-504) | (365-481)(505-506) |
| HG1014630 | 1 | (89-111) | (1-88)(112-165) |
| HG1014631 | 2 | (12-34)(450-472) | (1-11)(35-449)(473-513) |
| HG1014632 | 1 | (266-288) | (1-265)(289-314) |
| HG1014633 | 6 | (318-340)(347-369) | (1-317)(341-346)(370-418) |
| | | (419-438)(651-673) | (439-650)(674-677) |
| | | (678-697)(717-739) | (698-716)(740-749) |
| HG1014634 | 5 | (25-47)(57-79)(207-226) | (1-24)(48-56)(80-206) |
| | | (241-263)(265-287) | (227-240)(264-264) |
| | | | (288-427) |
| HG1014635 | 4 | (52-74)(78-100)(171-190) | (1-51)(75-77)(101-170) |
| | | (200-217) | (191-199)(218-511) |
| HG1014636 | 4 | (7-29)(78-100)(121-143) | (1-6)(30-77)(101-120) |
| | | (163-185) | (144-162)(186-220) |
| HG1014637 | 1 | (13-35) | (1-12)(36-373) |
| HG1014638 | 0 | | (1-1630) |
| HG1014639 | 1 | (209-231) | (1-208)(232-616) |
| HG1014640 | 1 | (656-678) | (1-655)(679-824) |
| HG1014641 | 1 | (664-686) | (1-663)(687-775) |
| HG1014642 | 2 | (7-29)(434-456) | (1-6)(30-433)(457-478) |
| HG1014643 | 1 | (109-126) | (1-108)(127-146) |
| HG1014644 | 7 | (406-428)(441-463) | (1-405)(429-440)(464-472) |
| | | (473-495)(508-530) | (496-507)(531-568) |
| | | (569-591)(603-625) | (592-602)(626-630) |
| | | (631-653) | (654-687) |
| HG1014645 | 1 | (1083-1105) | (1-1082)(1106-1238) |
| HG1014646 | 1 | (13-35) | (1-12)(36-344) |
| HG1014647 | 0 | | (1-2013) |
| HG1014692 | 0 | | (1-540) |
| HG1014693 | 1 | (664-686) | (1-663)(687-775) |
| HG1014694 | 4 | (166-188)(195-214) | (1-165)(189-194)(215-228) |
| | | (229-251)(369-391) | (252-368) |
| HG1014695 | 4 | (134-156)(163-182) | (1-133)(157-162)(183-196) |
| | | (197-219)(337-359) | (220-336)(360-361) |
| HG1014696 | 4 | (166-188)(195-214) | (1-165)(189-194)(215-228) |
| | | (229-251)(369-391) | (252-368)(392-393) |
| HG1014697 | 10 | (13-35)(103-122) | (1-12)(36-102)(123-142) |
| | | (143-165)(192-214) | (166-191)(215-234) |
| | | (235-257)(332-354) | (258-331)(355-360) |
| | | (361-380)(427-449) | (381-426)(450-460) |
| | | (461-483)(493-515) | (484-492)(516-526) |
| HG1014698 | 0 | | (1-147) |
| HG1014699 | 2 | (226-245)(252-274) | (1-225)(246-251)(275-279) |
| HG1014700 | 1 | (13-35) | (1-12)(36-136) |
| HG1014701 | 8 | (37-59)(69-88)(158-177) | (1-36)(60-68)(89-157) |
| | | (187-205)(217-239) | (178-186)(206-216) |
| | | (254-276)(281-303) | (240-253)(277-280) |
| | | (308-330) | (304-307)(331-355) |
| HG1014702 | 0 | | (1-374) |
| HG1014703 | 0 | | (1-358) |
| HG1014704 | 0 | | (1-528) |
| HG1014705 | 0 | | (1-1101) |
| HG1014706 | 0 | | (1-2863) |
| HG1014707 | 1 | (560-582) | (1-559)(583-998) |
| HG1014708 | 0 | | (1-1630) |
| HG1014709 | 0 | | (1-1806) |
| HG1014710 | 1 | (1243-1265) | (1-1242)(1266-1888) |
| HG1014711 | 7 | (405-427)(447-469) | (1-404)(428-446)(470-478) |
| | | (479-501)(514-536) | (502-513)(537-574) |
| | | (575-597)(609-631) | (598-608)(632-636) |
| | | (637-659) | (660-693) |
| HG1014712 | 2 | (21-43)(425-447) | (1-20)(44-424)(448-456) |
| HG1014713 | 1 | (543-565) | (1-542)(566-987) |
| HG1014714 | 1 | (1045-1067) | (1-1044)(1068-1200) |
| HG1014715 | 1 | (1300-1322) | (1-1299)(1323-1380) |
| HG1014716 | 4 | (41-63)(83-105)(126-148) | (1-40)(64-82)(106-125) |
| | | (194-216) | (149-193)(217-236) |
| HG1014717 | 4 | (13-35)(45-67)(88-110) | (1-12)(36-44)(68-87) |
| | | (157-179) | (111-156)(180-199) |
| HG1014718 | 0 | | (1-1811) |
| HG1014719 | 5 | (4-23)(35-57)(107-129) | (1-3)(24-34)(58-106) |
| | | (142-159)(169-191) | (130-141)(160-168) |
| | | | (192-232) |
| HG1014720 | 5 | (77-99)(112-134) | (1-76)(100-111)(135-183) |
| | | (184-206)(219-236) | (207-218)(237-245) |
| | | (246-268) | (269-309) |
| HG1014721 | 1 | (13-35) | (1-12)(36-372) |
| HG1014722 | 0 | | (1-1464) |
| HG1014723 | 1 | (1146-1168) | (1-1145)(1169-1215) |
| HG1014724 | 1 | (38-60) | (1-37)(61-484) |
| HG1014725 | 4 | (279-301)(308-327) | (1-278)(302-307)(328-341) |
| | | (342-364)(482-504) | (365-481)(505-506) |
| HG1014726 | 0 | | (1-1128) |
| HG1014727 | 0 | | (1-273) |
| HG1014728 | 1 | (417-439) | (1-416)(440-913) |
| HG1014729 | 1 | (417-439) | (1-416)(440-919) |
| HG1014730 | 0 | | (1-1405) |
| HG1014731 | 1 | (7-29) | (1-6)(30-331) |
| HG1014732 | 9 | (27-49)(70-92)(105-127) | (1-26)(50-69)(93-104) |
| | | (147-169)(184-206) | (128-146)(170-183) |
| | | (383-405)(409-431) | (207-382)(406-408) |
| | | (502-521)(531-548) | (432-501)(522-530) |
| | | | (549-842) |
| HG1014733 | 14 | (37-56)(63-85)(100-122) | (1-36)(57-62)(86-99) |
| | | (134-153)(168-190) | (123-133)(154-167) |
| | | (303-325)(345-367) | (191-302)(326-344) |
| | | (423-445)(449-471) | (368-422)(446-448) |
| | | (535-557)(970-992) | (472-534)(558-969) |
| | | (1012-1034) | (993-1011)(1035-1102) |
| | | (1103-1125) | (1126-1193)(1217-1527) |
| | | (1194-1216) | |
| HG1014734 | 13 | (37-56)(63-85)(100-122) | (1-36)(57-62)(86-99) |
| | | (134-153)(168-190) | (123-133)(154-167) |
| | | (303-325)(345-367) | (191-302)(326-344) |
| | | (423-445)(449-471) | (368-422)(446-448) |
| | | (535-557)(970-992) | (472-534)(558-969) |
| | | (1012-1034) | (993-1011)(1035-1102) |
| | | (1103-1125) | (1126-1238) |
| HG1014735 | 0 | | (1-947) |
| HG1014736 | 0 | | (1-774) |
| HG1014737 | 0 | | (1-528) |
| HG1014738 | 0 | | (1-707) |
| HG1014739 | 0 | | (1-309) |
| HG1014740 | 0 | | (1-93) |
| HG1014741 | 9 | (81-103)(124-146) | (1-80)(104-123)(147-158) |
| | | (159-181)(201-223) | (182-200)(224-237) |
| | | (238-260)(437-459) | (261-436)(460-462) |
| | | (463-485)(556-575) | (486-555)(576-584) |
| | | (585-602) | (603-896) |
| HG1014742 | 5 | (81-103)(124-146) | (1-80)(104-123)(147-158) |
| | | (159-181)(201-223) | (182-200)(224-237) |
| | | (238-260) | (261-283) |
| HG1014743 | 0 | | (1-337) |
| HG1014744 | 1 | (13-35) | (1-12)(36-344) |
| HG1014745 | 1 | (106-128) | (1-105)(129-552) |
| HG1014746 | 1 | (106-128) | (1-105)(129-621) |
| HG1014747 | 4 | (41-63)(83-105)(126-148) | (1-40)(64-82)(106-125) |
| | | (194-216) | (149-193)(217-236) |
| HG1014748 | 1 | (10-32) | (1-9)(33-74) |
| HG1014749 | 0 | | (1-59) |
| HG1014750 | 0 | | (1-562) |
| HG1014751 | 7 | (38-60)(91-110)(123-142) | (1-37)(61-90)(111-122) |
| | | (162-184)(196-218) | (143-161)(185-195) |
| | | (279-301)(355-377) | (219-278)(302-354) |
| | | | (378-433) |
| HG1014752 | 1 | (13-35) | (1-12)(36-344) |
| HG1014753 | 0 | | (1-186) |
| HG1014754 | 0 | | (1-186) |
| HG1014755 | 0 | | (1-82) |
| HG1014756 | 0 | | (1-62) |
| HG1014757 | 1 | (5-27) | (1-4)(28-39) |
| HG1014758 | 0 | | (1-1308) |
| HG1014759 | 1 | (199-221) | (1-198)(222-252) |
| HG1014760 | 4 | (52-71)(284-306) | (1-51)(72-283)(307-310) |
| | | (311-330)(350-372) | (331-349)(373-382) |
| HG1014761 | 0 | | (1-714) |
| HG1014762 | 0 | | (1-453) |
| HG1014763 | 1 | (626-648) | (1-625)(649-705) |
| HG1014764 | 0 | | (1-1729) |
| HG1014765 | 0 | | (1-108) |
| HG1014766 | 5 | (25-47)(57-79)(207-226) | (1-24)(48-56)(80-206) |
| | | (241-263)(265-287) | (227-240)(264-264) |
| | | | (288-427) |
| HG1014767 | 0 | | (1-24) |
| HG1014768 | 0 | | (1-83) |
| HG1014769 | 14 | (37-56)(63-85)(100-122) | (1-36)(57-62)(86-99) |
| | | (134-153)(168-190) | (123-133)(154-167) |
| | | (303-325)(345-367) | (191-302)(326-344) |
| | | (423-445)(449-471) | (368-422)(446-448) |
| | | (535-557)(970-992) | (472-534)(558-969) |
| | | (1012-1034) | (993-1011)(1035-1102) |
| | | (1103-1125) | (1126-1193)(1217-1514) |
| | | (1194-1216) | |
| HG1014770 | 1 | (433-455) | (1-432)(456-468) |
| HG1014771 | 0 | | (1-835) |
| HG1014772 | 0 | | (1-49) |
| HG1014773 | 0 | | (1-1715) |
| HG1014774 | 7 | (74-96)(117-139) | (1-73)(97-116)(140-183) |
| | | (184-206)(307-329) | (207-306)(330-332) |
| | | (333-355)(426-445) | (356-425)(446-454) |
| | | (455-472) | (473-766) |
| HG1014775 | 0 | | (1-240) |
| HG1014776 | 4 | (278-300)(307-326) | (1-277)(301-306)(327-340) |
| | | (341-363)(481-503) | (364-480)(504-505) |
| HG1014777 | 0 | | (1-66) |
| HG1014778 | 0 | | (1-41) |
| HG1014779 | 0 | | (1-60) |
| HG1014780 | 0 | | (1-117) |
| HG1014781 | 0 | | (1-716) |
| HG1014782 | 0 | | (1-656) |
| HG1014783 | 0 | | (1-1069) |
| HG1014784 | 1 | (37-59) | (1-36)(60-483) |
| HG1014785 | 1 | (42-64) | (1-41)(65-837) |
| HG1014786 | 0 | | (1-2147) |
| HG1014787 | 0 | | (1-1844) |
| HG1014788 | 9 | (35-57)(91-108)(113-135) | (1-34)(58-90)(109-112) |
| | | (139-158)(179-201) | (136-138)(159-178) |
| | | (216-235)(287-309) | (202-215)(236-286) |
| | | (329-348)(369-391) | (310-328)(349-368) |
| | | | (392-552) |
| HG1014789 | 2 | (12-34)(408-430) | (1-11)(35-407)(431-439) |
| HG1014790 | 0 | | (1-2851) |
| HG1014791 | 1 | (850-872) | (1-849)(873-971) |
| HG1014792 | 1 | (57-79) | (1-56)(80-503) |
| HG1014793 | 0 | | (1-625) |
| HG1014794 | 0 | | (1-399) |
| HG1014795 | 0 | | (1-391) |
| HG1014796 | 0 | | (1-657) |
| HG1014797 | 7 | (405-427)(447-469) | (1-404)(428-446)(470-478) |
| | | (479-501)(514-536) | (502-513)(537-574) |
| | | (575-597)(609-631) | (598-608)(632-636) |
| | | (637-659) | (660-693) |
| HG1014798 | 1 | (368-390) | (1-367)(391-461) |
| HG1014799 | 0 | | (1-364) |
| HG1014800 | 1 | (1083-1105) | (1-1082)(1106-1238) |
| HG1014801 | 1 | (576-598) | (1-575)(599-629) |
| HG1014802 | 0 | | (1-1464) |
| HG1014803 | 0 | | (1-707) |
| HG1014804 | 0 | | (1-1752) |
| HG1014805 | 0 | | (1-1822) |
| HG1014769 | 14 | (37-56)(63-85)(100-122) | (1-36)(57-62)(86-99) |
| | | (134-153)(168-190) | (123-133)(154-167) |
| | | (303-325)(345-367) | (191-302)(326-344) |
| | | (423-445)(449-471) | (368-422)(446-448) |
| | | (535-557)(970-992) | (472-534)(558-969) |
| | | (1012-1034) | (993-1011)(1035-1102) |
| | | (1103-1125) | (1126-1193)(1217-1514) |
| | | (1194-1216) | |
| HG1014770 | 1 | (433-455) | (1-432)(456-468) |
| HG1014771 | 0 | | (1-835) |
| HG1014772 | 0 | | (1-49) |
| HG1014773 | 0 | | (1-1715) |
| HG1014774 | 7 | (74-96)(117-139) | (1-73)(97-116)(140-183) |
| | | (184-206)(307-329) | (207-306)(330-332) |
| | | (333-355)(426-445) | (356-425)(446-454) |
| | | (455-472) | (473-766) |
| HG1014775 | 0 | | (1-240) |
| HG1014776 | 4 | (278-300)(307-326) | (1-277)(301-306)(327-340) |
| | | (341-363)(481-503) | (364-480)(504-505) |
| HG1014777 | 0 | | (1-66) |
| HG1014778 | 0 | | (1-41) |
| HG1014779 | 0 | | (1-60) |
| HG1014780 | 0 | | (1-117) |
| HG1014781 | 0 | | (1-716) |
| HG1014782 | 0 | | (1-656) |
| HG1014783 | 0 | | (1-1069) |
| HG1014784 | 1 | (37-59) | (1-36)(60-483) |
| HG1014785 | 1 | (42-64) | (1-41)(65-837) |
| HG1014786 | 0 | | (1-2147) |
| HG1014787 | 0 | | (1-1844) |
| HG1014788 | 9 | (35-57)(91-108)(113-135) | (1-34)(58-90)(109-112) |
| | | (139-158)(179-201) | (136-138)(159-178) |
| | | (216-235)(287-309) | (202-215)(236-286) |
| | | (329-348)(369-391) | (310-328)(349-368) |
| | | | (392-552) |
| HG1014789 | 2 | (12-34)(408-430) | (1-11)(35-407)(431-439) |
| HG1014790 | 0 | | (1-2851) |
| HG1014791 | 1 | (850-872) | (1-849)(873-971) |
| HG1014792 | 1 | (57-79) | (1-56)(80-503) |
| HG1014793 | 0 | | (1-625) |
| HG1014794 | 0 | | (1-399) |
| HG1014795 | 0 | | (1-391) |
| HG1014796 | 0 | | (1-657) |
| HG1014797 | 7 | (405-427)(447-469) | (1-404)(428-446)(470-478) |
| | | (479-501)(514-536) | (502-513)(537-574) |
| | | (575-597)(609-631) | (598-608)(632-636) |
| | | (637-659) | (660-693) |
| HG1014798 | 1 | (368-390) | (1-367)(391-461) |
| HG1014799 | 0 | | (1-364) |
| HG1014800 | 1 | (1083-1105) | (1-1082)(1106-1238) |
| HG1014801 | 1 | (576-598) | (1-575)(599-629) |
| HG1014802 | 0 | | (1-1464) |
| HG1014803 | 0 | | (1-707) |
| HG1014804 | 0 | | (1-1752) |
| HG1014805 | 0 | | (1-1822) |
| HG1014806 | 0 | | (1-1805) |
| HG1014807 | 4 | (279-301)(308-327) | (1-278)(302-307)(328-341) |
| | | (342-364)(482-504) | (365-481)(505-506) |
| HG1014808 | 0 | | (1-76) |
| HG1014809 | 1 | (89-111) | (1-88)(112-147) |
| HG1014810 | 2 | (7-24)(1083-1105) | (1-6)(25-1082)(1106-1238) |
| HG1014811 | 0 | | (1-528) |
| HG1014812 | 0 | | (1-501) |
| HG1014813 | 5 | (48-70)(77-99)(150-169) | (1-47)(71-76)(100-149) |
| | | (382-404)(409-428) | (170-381)(405-408) |
| | | | (429-433) |
| HG1014814 | 1 | (199-221) | (1-198)(222-252) |
| HG1014815 | 1 | (1045-1067) | (1-1044)(1068-1200) |
| HG1014816 | 0 | | (1-1941) |
| HG1014817 | 2 | (21-43)(425-447) | (1-20)(44-424)(448-456) |
| HG1014818 | 1 | (376-398) | (1-375)(399-455) |
| HG1014819 | 0 | | (1-100) |
| HG1014820 | 0 | | (1-1068) |
| HG1014821 | 0 | | (1-543) |
| HG1014822 | 1 | (1068-1090) | (1-1067)(1091-1223) |
| HG1014823 | 0 | | (1-1238) |
| HG1014824 | 0 | | (1-459) |
| HG1014825 | 2 | (7-29)(55-77) | (1-6)(30-54)(78-94) |
| HG1014826 | 0 | | (1-1129) |
| HG1014827 | 0 | | (1-600) |
| HG1014828 | 0 | | (1-832) |
| HG1014829 | 9 | (63-82)(89-111)(126-148) | (1-62)(83-88)(112-125) |
| | | (160-179)(194-216) | (149-159)(180-193) |
| | | (329-351)(371-393) | (217-328)(352-370) |
| | | (449-471)(475-497) | (394-448)(472-474) |
| | | | (498-598) |
| HG1014830 | 1 | (1300-1322) | (1-1299)(1323-1380) |
| HG1014831 | 0 | | (1-347) |
| HG1014832 | 1 | (1253-1275) | (1-1252)(1276-1898) |
| HG1014833 | 1 | (199-221) | (1-198)(222-252) |
| HG1014834 | 0 | | (1-119) |
| HG1014835 | 0 | | (1-472) |
| HG1014836 | 0 | | (1-61) |
| HG1014837 | 14 | (37-56)(63-85)(100-122) | (1-36)(57-62)(86-99) |
| | | (134-153)(168-190) | (123-133)(154-167) |
| | | (303-325)(345-367) | (191-302)(326-344) |
| | | (423-445)(449-471) | (368-422)(446-448) |
| | | (535-557)(970-992) | (472-534)(558-969) |
| | | (1012-1034) | (993-1011)(1035-1102) |
| | | (1103-1125) | (1126-1193)(1217-1527) |
| | | (1194-1216) | |
| HG1014838 | 1 | (368-390) | (1-367)(391-461) |
| HG1014839 | 0 | | (1-1752) |
| HG1014840 | 1 | (1009-1031) | (1-1008)(1032-1067) |
| HG1014841 | 0 | | (1-1805) |
| HG1014842 | 0 | | (1-181) |
| HG1014843 | 2 | (7-26)(36-58) | (1-6)(27-35)(59-141) |
| HG1014844 | 1 | (337-359) | (1-336)(360-430) |
| HG1014845 | 1 | (339-361) | (1-338)(362-373) |
| HG1014846 | 0 | | (1-890) |
| HG1014847 | 0 | | (1-94) |
| HG1014848 | 0 | | (1-284) |
| HG1014849 | 14 | (37-56)(63-85)(100-122) | (1-36)(57-62)(86-99) |
| | | (134-153)(168-190) | (123-133)(154-167) |
| | | (299-321)(346-368) | (191-298)(322-345) |
| | | (424-446)(450-472) | (369-423)(447-449) |
| | | (536-558)(971-993) | (473-535)(559-970) |
| | | (1013-1035) | (994-1012)(1036-1103) |
| | | (1104-1126) | (1127-1194)(1218-1528) |
| | | (1195-1217) | |
| HG1014850 | 11 | (179-201)(216-238) | (1-178)(202-215)(239-292) |
| | | (293-315)(320-342) | (316-319)(343-395) |
| | | (396-418)(428-447) | (419-427)(448-856) |
| | | (857-879)(914-936) | (880-913)(937-992) |
| | | (993-1010) | (1011-1013)(1037-1096) |
| | | (1014-1036) | (1120-1437) |
| | | (1097-1119) | |
| HG1014851 | 0 | | (1-1461) |
| HG1014852 | 14 | (37-56)(63-85)(100-122) | (1-36)(57-62)(86-99) |
| | | (134-153)(168-190) | (123-133)(154-167) |
| | | (303-325)(345-367) | (191-302)(326-344) |
| | | (423-445)(449-471) | (368-422)(446-448) |
| | | (535-557)(970-992) | (472-534)(558-969) |
| | | (1012-1034) | (993-1011)(1035-1102) |
| | | (1103-1125) | (1126-1193)(1217-1527) |
| | | (1194-1216) | |
| HG1014853 | 7 | (405-427)(447-469) | (1-404)(428-446)(470-478) |
| | | (479-501)(514-536) | (502-513)(537-574) |
| | | (575-597)(609-631) | (598-608)(632-636) |
| | | (637-659) | (660-693) |
| HG1014854 | 3 | (2-21)(46-68)(113-132) | (1-1)(22-45)(69-112) |
| | | | (133-134) |
| HG1014855 | 1 | (538-560) | (1-537)(561-981) |
| HG1014856 | 0 | | (1-79) |
| HG1014857 | 11 | (179-201)(216-238) | (1-178)(202-215)(239-292) |
| | | (293-315)(320-342) | (316-319)(343-395) |
| | | (396-418)(428-447) | (419-427)(448-856) |
| | | (857-879)(914-936) | (880-913)(937-992) |
| | | (993-1010) | (1011-1013)(1037-1096) |
| | | (1014-1036) | (1120-1437) |
| | | (1097-1119) | |
| HG1014858 | 0 | | (1-285) |
| HG1014859 | 0 | | (1-595) |
| HG1014860 | 1 | (1015-1037) | (1-1014)(1038-1073) |
| HG1014861 | 0 | | (1-249) |
| HG1014862 | 0 | | (1-21) |
| HG1014863 | 0 | | (1-47) |
| HG1014864 | 0 | | (1-161) |
| HG1014865 | 1 | (80-97) | (1-79)(98-117) |
| HG1014866 | 0 | | (1-115) |
| HG1014867 | 0 | | (1-226) |
| HG1014868 | 0 | | (1-2298) |
| HG1014869 | 0 | | (1-329) |
| HG1014870 | 1 | (417-439) | (1-416)(440-875) |
| HG1014871 | 1 | (266-288) | (1-265)(289-314) |
| HG1014872 | 0 | | (1-306) |
| HG1014873 | 1 | (417-439) | (1-416)(440-876) |
| HG1014874 | 0 | | (1-321) |
| HG1014875 | 0 | | (1-417) |
| HG1014876 | 5 | (366-388)(423-445) | (1-365)(389-422)(446-501) |
| | | (502-519)(523-545) | (520-522)(546-605) |
| | | (606-628) | (629-946) |
| HG1014877 | 0 | | (1-353) |
| HG1014878 | 0 | | (1-1864) |
| HG1014879 | 0 | | (1-501) |
| HG1014880 | 0 | | (1-208) |
| HG1014881 | 9 | (38-57)(64-86)(101-123) | (1-37)(58-63)(87-100) |
| | | (135-154)(169-191) | (124-134)(155-168) |
| | | (304-326)(346-368) | (192-303)(327-345) |
| | | (424-446)(450-472) | (369-423)(447-449) |
| | | | (473-573) |
| HG1014882 | 1 | (628-650) | (1-627)(651-686) |
| HG1014883 | 11 | (179-201)(216-238) | (1-178)(202-215)(239-292) |
| | | (293-315)(320-342) | (316-319)(343-395) |
| | | (396-418)(428-447) | (419-427)(448-856) |
| | | (857-879)(914-936) | (880-913)(937-996) |
| | | (997-1019) | (1020-1053)(1077-1082) |
| | | (1054-1076) | (1106-1394) |
| | | | (1083-1105) |
| HG1014884 | 2 | (12-34)(466-488) | (1-11)(35-465)(489-529) |
| HG1014885 | 1 | (546-568) | (1-545)(569-1191) |
| HG1014886 | 0 | | (1-1601) |
| HG1014887 | 1 | (38-60) | (1-37)(61-484) |
| HG1014888 | 4 | (13-35)(45-67)(88-110) | (1-12)(36-44)(68-87) |
| | | (157-179) | (111-156)(180-199) |
| HG1014889 | 0 | | (1-224) |
| HG1014890 | 1 | (1083-1105) | (1-1082)(1106-1238) |
| HG1014891 | 1 | (33-55) | (1-32)(56-478) |
| HG1014892 | 1 | (25-42) | (1-24)(43-135) |
| HG1014893 | 1 | (1300-1322) | (1-1299)(1323-1380) |
| HG1014894 | 0 | | (1-102) |
| HG1014895 | 0 | | (1-497) |
| HG1014896 | 14 | (37-56)(63-85)(100-122) | (1-36)(57-62)(86-99) |
| | | (134-153)(168-190) | (123-133)(154-167) |
| | | (303-325)(345-367) | (191-302)(326-344) |
| | | (423-445)(449-471) | (368-422)(446-448) |
| | | (535-557)(970-992) | (472-534)(558-969) |
| | | (1012-1034) | (993-1011)(1035-1102) |
| | | (1103-1125) | (1126-1193)(1217-1527) |
| | | (1194-1216) | |
| HG1014897 | 1 | (711-733) | (1-710)(734-964) |
| HG1014898 | 0 | | (1-351) |
| HG1014899 | 11 | (179-201)(216-238) | (1-178)(202-215)(239-292) |
| | | (293-315)(320-342) | (316-319)(343-395) |
| | | (396-418)(428-447) | (419-427)(448-856) |
| | | (857-879)(914-936) | (880-913)(937-992) |
| | | (993-1010) | (1011-1013)(1037-1096) |
| | | (1014-1036) | (1120-1437) |
| | | (1097-1119) | |
| HG1014900 | 2 | (12-34)(466-488) | (1-11)(35-465)(489-529) |

TABLE 4
Pfam Coordinates

| FP ID | Protein ID | Pfam | Pfam Coords. |
|---|---|---|---|
| HG1014563 | 730241:473936 | DDOST_48 kD | (26-455) |
| HG1014564 | proteinkinase98A:proteinkinase98B | EPH_lbd | (20-197) |
| HG1014564 | proteinkinase98A:proteinkinase98B | fn3 | (325-421) |
| HG1014564 | proteinkinase98A:proteinkinase98B | fn3 | (436-520) |
| HG1014564 | proteinkinase98A:proteinkinase98B | pkinase | (621-880) |
| HG1014564 | proteinkinase98A:proteinkinase98B | SAM | (911-975) |
| HG1014565 | NP_006501:NM_006510 | zf-C3HC4 | (16-56) |
| HG1014565 | NP_006501:NM_006510 | SPRY | (368-493) |
| HG1014565 | NP_006501:NM_006510 | zf-B_box | (93-132) |
| HG1014566 | 2738927:2738926 | no_pfam | |
| HG1014567 | 3646130:3646129 | ATP-bind | (8-248) |
| HG1014568 | 7512502:7512502_genewise | GDPD | (148-389) |
| HG1014568 | 7512502:7512502_genewise | GDPD | (70-87) |
| HG1014569 | 88918:550030 | ig | (160-217) |
| HG1014569 | 88918:550030 | ig | (252-301) |
| HG1014569 | 88918:550030 | ig | (341-398) |
| HG1014570 | 4240243:4240242 | no_pfam | |
| HG1014571 | NP_056438:NM_015623 | no_pfam | |
| HG1014572 | NP_001703:NM_001712 | ig | (160-217) |
| HG1014572 | NP_001703:NM_001712 | ig | (252-301) |
| HG1014572 | NP_001703:NM_001712 | ig | (341-398) |
| HG1014573 | NP_003703:NM_003712 | PAP2 | (107-248) |
| HG1014574 | proteinkinase16A:proteinkinase16B | Activin_recp | (20-107) |
| HG1014574 | proteinkinase16A:proteinkinase16B | pkinase | (208-495) |
| HG1014575 | 602434:602433 | SNF | (266-426) |
| HG1014575 | 602434:602433 | SNF | (461-507) |
| HG1014575 | 602434:602433 | SNF | (532-567) |
| HG1014575 | 602434:602433 | SNF | (596-694) |
| HG1014576 | NP_005177:NM_005186 | Calpain_III | (365-522) |
| HG1014576 | NP_005177:NM_005186 | Peptidase_C2 | (55-354) |
| HG1014576 | NP_005177:NM_005186 | efhand | (619-647) |
| HG1014577 | 3327124:3327123 | ENTH | (43-162) |
| HG1014577 | 3327124:3327123 | I_LWEQ | (834-1027) |
| HG1014578 | NP_001934:NM_001943 | cadherin | (163-262) |
| HG1014578 | NP_001934:NM_001943 | cadherin | (276-377) |
| HG1014578 | NP_001934:NM_001943 | cadherin | (395-489) |
| HG1014578 | NP_001934:NM_001943 | cadherin | (93-149) |
| HG1014579 | NP_002417:NM_002426 | Peptidase_M10 | (102-208) |
| HG1014579 | NP_002417:NM_002426 | peptidase_M10_N | (17-96) |
| HG1014579 | NP_002417:NM_002426 | hemopexin | (288-330) |
| HG1014579 | NP_002417:NM_002426 | hemopexin | (332-375) |
| HG1014579 | NP_002417:NM_002426 | hemopexin | (380-427) |
| HG1014579 | NP_002417:NM_002426 | hemopexin | (429-470) |
| HG1014580 | NP_002236:NM_002245 | no_pfam | |
| HG1014581 | 3882213:3882212 | no_pfam | |
| HG1014582 | 2439970:2439969 | ABC_membrane | (2-202) |
| HG1014582 | 2439970:2439969 | ABC_tran | (274-457) |
| HG1014583 | NP_005859:NM_005868 | SNARE | (31-93) |
| HG1014584 | NP_005778:NM_005787 | ALG3 | (45-406) |
| HG1014585 | 887368:887367 | EMP24_GP25L | (25-114) |
| HG1014586 | NP_055688:NM_014873 | no_pfam | |
| HG1014587 | 7513004:3043577 | no_pfam | |
| HG1014588 | 20521660:20521659 | DEAD | (1208-1418) |
| HG1014588 | 20521660:20521659 | Sec63 | (1699-2015) |
| HG1014588 | 20521660:20521659 | DEAD | (361-580) |
| HG1014588 | 20521660:20521659 | helicase_C | (664-750) |
| HG1014588 | 20521660:20521659 | Sec63 | (868-1177) |
| HG1014589 | 12230553:1665780 | no_pfam | |
| HG1014590 | NP_059984:NM_017514 | TIG | (1023-1122) |
| HG1014590 | NP_059984:NM_017514 | TIG | (1125-1200) |
| HG1014590 | NP_059984:NM_017514 | Sema | (33-471) |
| HG1014590 | NP_059984:NM_017514 | PSI | (490-540) |
| HG1014590 | NP_059984:NM_017514 | PSI | (637-684) |
| HG1014590 | NP_059984:NM_017514 | PSI | (785-838) |
| HG1014590 | NP_059984:NM_017514 | TIG | (840-933) |
| HG1014590 | NP_059984:NM_017514 | TIG | (935-1020) |
| HG1014591 | NP_002831:NM_002840 | Y_phosphatase | (1365-1596) |
| HG1014591 | NP_002831:NM_002840 | ig | (139-199) |
| HG1014591 | NP_002831:NM_002840 | Y_phosphatase | (1654-1887) |
| HG1014591 | NP_002831:NM_002840 | ig | (236-290) |
| HG1014591 | NP_002831:NM_002840 | fn3 | (309-391) |
| HG1014591 | NP_002831:NM_002840 | ig | (37-99) |
| HG1014591 | NP_002831:NM_002840 | fn3 | (403-490) |
| HG1014591 | NP_002831:NM_002840 | fn3 | (502-584) |
| HG1014591 | NP_002831:NM_002840 | fn3 | (596-686) |
| HG1014591 | NP_002831:NM_002840 | fn3 | (698-799) |
| HG1014591 | NP_002831:NM_002840 | fn3 | (811-894) |
| HG1014591 | NP_002831:NM_002840 | fn3 | (905-990) |
| HG1014592 | 3043698:3043697 | no_pfam | |
| HG1014593 | 14133205:14133204 | ig | (180-268) |
| HG1014593 | 14133205:14133204 | ig | (315-398) |
| HG1014593 | 14133205:14133204 | ig | (445-533) |
| HG1014593 | 14133205:14133204 | ig | (55-142) |
| HG1014593 | 14133205:14133204 | ig | (714-804) |
| HG1014593 | 14133205:14133204 | ig | (851-940) |
| HG1014594 | NP_055453:NM_014638 | no_pfam | |
| HG1014595 | NP_064422:NM_020038 | no_pfam | |
| HG1014596 | 1580781:1580780 | Beach | (1438-1715) |
| HG1014596 | 1580781:1580780 | WD40 | (1855-1896) |
| HG1014597 | 2136093:403386 | F5_F8_type_C | (34-182) |
| HG1014597 | 2136093:403386 | pkinase | (610-905) |
| HG1014598 | NP_005119:NM_005128 | Dopey_N | (2-314) |
| HG1014599 | 559330:559329 | no_pfam | |
| HG1014600 | 1665787:1665786 | no_pfam | |
| HG1014601 | NP_003307:NM_003316 | zf-C3HC4 | (1957-1996) |
| HG1014602 | NP_055098:NM_014283 | no_pfam | |
| HG1014603 | 21903712:22004648 | DUF857 | (1128-1236) |
| HG1014603 | 21903712:22004648 | DUF857 | (297-406) |
| HG1014603 | 21903712:22004648 | Zn_carbOpept | (501-684) |
| HG1014603 | 21903712:22004648 | Zn_carbOpept | (56-270) |
| HG1014603 | 21903712:22004648 | DUF857 | (709-818) |
| HG1014603 | 21903712:22004648 | Zn_carbOpept | (931-1109) |
| HG1014604 | 403460:403459 | no_pfam | |
| HG1014605 | 20140021:1888315 | DPPIV_N_term | (42-548) |
| HG1014605 | 20140021:1888315 | Peptidase_S9 | (552-629) |
| HG1014606 | 2996578:2996577 | Alg6_Alg8 | (25-521) |
| HG1014607 | 729008:306474 | F5_F8_type_C | (34-182) |
| HG1014607 | 729008:306474 | pkinase | (610-905) |
| HG1014608 | NP_001296:NM_001305 | PMP22_Claudin | (4-181) |
| HG1014609 | NP_066192:NM_020982 | PMP22_Claudin | (4-181) |
| HG1014610 | NP_006293:NM_006302 | Glyco_hydro_63 | (50-836) |
| HG1014611 | 4691263:4557422 | GDA1_CD39 | (430-480) |
| HG1014611 | 4691263:4557422 | GDA1_CD39 | (93-332) |
| HG1014612 | NP_006806:NM_006815 | EMP24_GP25L | (5-201) |
| HG1014613 | NP_036380:NM_012248 | AIRS | (115-239) |
| HG1014613 | NP_036380:NM_012248 | AIRS_C | (243-418) |
| HG1014614 | 5459516:5459515 | PEMT | (2-199) |
| HG1014615 | proteinkinase99A:proteinkinase99B | fn3 | (340-435) |
| HG1014615 | proteinkinase99A:proteinkinase99B | EPH_lbd | (39-212) |
| HG1014615 | proteinkinase99A:proteinkinase99B | fn3 | (453-535) |
| HG1014615 | proteinkinase99A:proteinkinase99B | pkinase | (633-892) |
| HG1014615 | proteinkinase99A:proteinkinase99B | SAM | (923-987) |
| HG1014616 | NP_055557:NM_014742 | EMP70 | (37-583) |
| HG1014617 | 4009517:4009516 | T4_deiodinase | (11-269) |
| HG1014618 | 1220309:1220308 | VKG_Carbox | (2-668) |
| HG1014619 | NP_005679:NM_005688 | ABC_tran | (1220-1403) |
| HG1014619 | NP_005679:NM_005688 | ABC_membrane | (179-447) |
| HG1014619 | NP_005679:NM_005688 | ABC_tran | (588-759) |
| HG1014619 | NP_005679:NM_005688 | ABC_membrane | (860-1147) |
| HG1014620 | NP_004985:NM_004994 | Peptidase_M10 | (109-215) |
| HG1014620 | NP_004985:NM_004994 | fn2 | (230-271) |
| HG1014620 | NP_004985:NM_004994 | Peptidase_M10_N | (26-103) |
| HG1014620 | NP_004985:NM_004994 | fn2 | (288-329) |
| HG1014620 | NP_004985:NM_004994 | fn2 | (347-388) |
| HG1014620 | NP_004985:NM_004994 | PT | (472-507) |
| HG1014620 | NP_004985:NM_004994 | hemopexin | (521-565) |
| HG1014620 | NP_004985:NM_004994 | hemopexin | (567-608) |
| HG1014620 | NP_004985:NM_004994 | hemopexin | (613-659) |
| HG1014620 | NP_004985:NM_004994 | hemopexin | (661-704) |
| HG1014621 | 1478281:1478280 | SDF | (54-485) |
| HG1014622 | NP_055759:NM_014944 | cadherin | (169-258) |
| HG1014623 | NP_066925:NM_021102 | Kunitz_BPTI | (133-183) |
| HG1014623 | NP_066925:NM_021102 | Kunitz_BPTI | (38-88) |
| HG1014624 | NP_000201:NM_000210 | integrin_A | (1038-1052) |
| HG1014624 | NP_000201:NM_000210 | FG-GAP | (316-367) |
| HG1014624 | NP_000201:NM_000210 | FG-GAP | (378-422) |
| HG1014624 | NP_000201:NM_000210 | FG-GAP | (432-474) |
| HG1014625 | NP_006661:NM_006670 | LRR | (235-258) |
| HG1014625 | NP_006661:NM_006670 | LRRCT | (294-345) |
| HG1014625 | NP_006661:NM_006670 | LRRNT | (61-90) |
| HG1014626 | NP_000204:NM_000213 | fn3 | (1127-1208) |
| HG1014626 | NP_000204:NM_000213 | fn3 | (1220-1310) |
| HG1014626 | NP_000204:NM_000213 | fn3 | (1528-1612) |
| HG1014626 | NP_000204:NM_000213 | fn3 | (1641-1728) |
| HG1014626 | NP_000204:NM_000213 | integrin_B | (37-455) |
| HG1014626 | NP_000204:NM_000213 | Calx-beta | (979-1084) |
| HG1014627 | NP_005767:NM_005776 | Cornichon | (6-136) |
| HG1014628 | 3288487:3288486 | Collagen | (263-290) |
| HG1014628 | 3288487:3288486 | Collagen | (291-338) |
| HG1014629 | 13124728:2285960 | Neur_chan_memb | (284-379) |
| HG1014629 | 13124728:2285960 | Neur_chan_memb | (475-500) |
| HG1014629 | 13124728:2285960 | Neur_chan_LBD | (71-277) |
| HG1014630 | 239160:239159 | no_pfam | |
| HG1014631 | NP_003701:NM_003710 | Kunitz_BPTI | (250-300) |
| HG1014631 | NP_003701:NM_003710 | ldl_recept_a | (317-355) |
| HG1014631 | NP_003701:NM_003710 | Kunitz_BPTI | (375-425) |
| HG1014632 | NP_002345:NM_002354 | thyroglobulin_1 | (66-135) |
| HG1014633 | NP_036451:NM_012319 | Zip | (316-534) |
| HG1014633 | NP_036451:NM_012319 | Zip | (548-737) |
| HG1014634 | NP_002241:NM_002250 | SK_channel | (12-121) |
| HG1014634 | NP_002241:NM_002250 | CaMBD | (304-377) |
| HG1014635 | 3387977:3387976 | ABC_membrane | (18-213) |
| HG1014635 | 3387977:3387976 | ABC_tran | (285-469) |
| HG1014636 | NP_001297:NM_001306 | PMP22_Claudin | (3-180) |
| HG1014637 | 3132896:3132895 | Galactosyl_T_2 | (97-367) |
| HG1014638 | 20521832:20521831 | PDZ | (1004-1092) |
| HG1014638 | 20521832:20521831 | PDZ | (1100-1188) |
| HG1014638 | 20521832:20521831 | PDZ | (728-814) |
| HG1014638 | 20521832:20521831 | PDZ | (871-949) |
| HG1014639 | NP_003830:NM_003839 | TNFR_c6 | (34-68) |
| HG1014640 | NP_001100:NM_001109 | Reprolysin | (200-400) |
| HG1014640 | NP_001100:NM_001109 | disintegrin | (417-492) |
| HG1014640 | NP_001100:NM_001109 | Pep_M12B_propep | (71-185) |
| HG1014641 | NP_055080:NM_014265 | Reprolysin | (204-399) |
| HG1014641 | NP_055080:NM_014265 | disintegrin | (416-491) |
| HG1014641 | NP_055080:NM_014265 | Pep_M12B_propep | (71-189) |
| HG1014642 | NP_005497:NM_005506 | CD36 | (2-439) |
| HG1014643 | NP_006685:NM_006694 | JTB | (1-146) |
| HG1014644 | 4456467:4456466 | GPS | (342-394) |
| HG1014644 | 4456467:4456466 | 7tm_2 | (400-659) |
| HG1014645 | NP_002217:NM_002226 | DSL | (178-240) |
| HG1014645 | NP_002217:NM_002226 | EGF | (311-344) |
| HG1014645 | NP_002217:NM_002226 | EGF | (351-382) |
| HG1014645 | NP_002217:NM_002226 | EGF | (389-420) |
| HG1014645 | NP_002217:NM_002226 | EGF | (427-458) |
| HG1014645 | NP_002217:NM_002226 | EGF | (465-495) |
| HG1014645 | NP_002217:NM_002226 | EGF | (502-533) |
| HG1014645 | NP_002217:NM_002226 | EGF | (540-571) |
| HG1014645 | NP_002217:NM_002226 | EGF | (640-671) |
| HG1014645 | NP_002217:NM_002226 | EGF | (678-709) |
| HG1014645 | NP_002217:NM_002226 | EGF | (716-747) |
| HG1014645 | NP_002217:NM_002226 | EGF | (755-786) |
| HG1014645 | NP_002217:NM_002226 | EGF | (793-824) |
| HG1014645 | NP_002217:NM_002226 | EGF | (831-862) |
| HG1014646 | NP_003769:NM_003778 | Galactosyl_T_2 | (77-344) |
| HG1014647 | 1504030:1504029 | no_pfam | |
| HG1014692 | NP_068547:NM_021777 | Reprolysin | (204-399) |
| HG1014692 | NP_068547:NM_021777 | disintegrin | (416-491) |
| HG1014692 | NP_068547:NM_021777 | Pep_M12B_propep | (71-189) |
| HG1014693 | NP_068548:NM_021778 | Reprolysin | (204-399) |
| HG1014693 | NP_068548:NM_021778 | disintegrin | (416-491) |
| HG1014693 | NP_068548:NM_021778 | Pep_M12B_propep | (71-189) |
| HG1014694 | NP_068819:NM_021984 | Neur_chan_LED | (1-164) |
| HG1014694 | NP_068819:NM_021984 | Neur_chan_memb | (171-266) |
| HG1014694 | NP_068819:NM_021984 | Neur_chan_memb | (362-387) |
| HG1014695 | NP_068822:NM_021987 | Neur_chan_memb | (139-234) |
| HG1014695 | NP_068822:NM_021987 | Neur_chan_LBD | (14-132) |
| HG1014695 | NP_068822:NM_021987 | Neur_chan_memb | (330-355) |
| HG1014696 | NP_068830:NM_021990 | Neur_chan_LED | (1-164) |
| HG1014696 | NP_068830:NM_021990 | Neur_chan_memb | (171-266) |
| HG1014696 | NP_068830:NM_021990 | Neur_chan_memb | (362-387) |
| HG1014697 | NP_076984:NM_024079 | Alg6_Alg8 | (19-515) |
| HG1014698 | NP_079327:NM_025051 | no_pfam | |
| HG1014699 | NP_108648:NM_030658 | no_pfam | |
| HG1014700 | NP_085076:NM_030587 | no_pfam | |
| HG1014701 | NP_055954:NM_015139 | no_pfam | |
| HG1014702 | NP_009197:NM_007266 | ATP-bind | (24-264) |
| HG1014703 | NP_112212:NM_030950 | zf-C3HC4 | (16-56) |
| HG1014703 | NP_112212:NM_030950 | zf-B_box | (93-132) |
| HG1014704 | NP_073572:NM_022735 | no_pfam | |
| HG1014705 | NP_079461:NM_025185 | ank | (134-166) |
| HG1014705 | NP_079461:NM_025185 | ank | (167-199) |
| HG1014705 | NP_079461:NM_025185 | ank | (18-50) |
| HG1014705 | NP_079461:NM_025185 | ank | (200-232) |
| HG1014705 | NP_079461:NM_025185 | ank | (233-265) |
| HG1014705 | NP_079461:NM_025185 | ank | (266-298) |
| HG1014705 | NP_079461:NM_025185 | ank | (51-74) |
| HG1014706 | NP_006717:NM_006726 | Beach | (2212-2489) |
| HG1014706 | NP_006717:NM_006726 | WD40 | (2629-2670) |
| HG1014707 | NP_004434:NM_004443 | fn3 | (340-435) |
| HG1014707 | NP_004434:NM_004443 | EPH_lbd | (39-212) |
| HG1014707 | NP_004434:NM_004443 | fn3 | (453-535) |
| HG1014707 | NP_004434:NM_004443 | pkinase | (633-892) |
| HG1014707 | NP_004434:NM_004443 | SAM | (923-987) |
| HG1014708 | NP_056171:NM_015356 | PDZ | (1004-1092) |
| HG1014708 | NP_056171:NM_015356 | PDZ | (1100-1188) |
| HG1014708 | NP_056171:NM_015356 | PDZ | (728-814) |
| HG1014708 | NP_056171:NM_015356 | PDZ | (871-949) |
| HG1014709 | NP_001845:NM_001854 | Collagen | (1039-1095) |
| HG1014709 | NP_001845:NM_001854 | Collagen | (1096-1155) |
| HG1014709 | NP_001845:NM_001854 | Collagen | (1156-1215) |
| HG1014709 | NP_001845:NM_001854 | Collagen | (1219-1278) |
| HG1014709 | NP_001845:NM_001854 | Collagen | (1279-1330) |
| HG1014709 | NP_001845:NM_001854 | Collagen | (1333-1392) |
| HG1014709 | NP_001845:NM_001854 | Collagen | (1393-1452) |
| HG1014709 | NP_001845:NM_001854 | Collagen | (1462-1521) |
| HG1014709 | NP_001845:NM_001854 | COLFI | (1593-1804) |
| HG1014709 | NP_001845:NM_001854 | TSPN | (38-229) |
| HG1014709 | NP_001845:NM_001854 | Collagen | (442-490) |
| HG1014709 | NP_001845:NM_001854 | Collagen | (528-579) |
| HG1014709 | NP_001845:NM_001854 | Collagen | (583-642) |
| HG1014709 | NP_001845:NM_001854 | Collagen | (643-702) |
| HG1014709 | NP_001845:NM_001854 | Collagen | (703-750) |
| HG1014709 | NP_001845:NM_001854 | Collagen | (751-810) |
| HG1014709 | NP_001845:NM_001854 | Collagen | (811-870) |
| HG1014709 | NP_001845:NM_001854 | Collagen | (874-933) |
| HG1014709 | NP_001845:NM_001854 | Collagen | (934-980) |
| HG1014709 | NP_001845:NM_001854 | Collagen | (982-1037) |
| HG1014710 | NP_569707:NM_130440 | Y_phosphatase | (1356-1587) |
| HG1014710 | NP_569707:NM_130440 | ig | (139-199) |
| HG1014710 | NP_569707:NM_130440 | Y_phosphatase | (1645-1878) |
| HG1014710 | NP_569707:NM_130440 | ig | (236-290) |
| HG1014710 | NP_569707:NM_130440 | fn3 | (309-391) |
| HG1014710 | NP_569707:NM_130440 | ig | (37-99) |
| HG1014710 | NP_569707:NM_130440 | fn3 | (403-490) |
| HG1014710 | NP_569707:NM_130440 | fn3 | (502-584) |
| HG1014710 | NP_569707:NM_130440 | fn3 | (596-686) |
| HG1014710 | NP_569707:NM_130440 | fn3 | (698-790) |
| HG1014710 | NP_569707:NM_130440 | fn3 | (802-885) |
| HG1014710 | NP_569707:NM_130440 | fn3 | (896-981) |
| HG1014711 | NP_005673:NM_005682 | GPS | (342-394) |
| HG1014711 | NP_005673:NM_005682 | 7tm_2 | (400-665) |
| HG1014712 | NP_005207:NM_005216 | DDOST_48 kD | (26-455) |
| HG1014713 | NP_004433:NM_004442 | EPH_lbd | (20-197) |
| HG1014713 | NP_004433:NM_004442 | fn3 | (325-421) |
| HG1014713 | NP_004433:NM_004442 | fn3 | (436-520) |
| HG1014713 | NP_004433:NM_004442 | pkinase | (622-881) |
| HG1014713 | NP_004433:NM_004442 | SAM | (912-976) |
| HG1014714 | NP_660142:NM_145159 | DSL | (178-240) |
| HG1014714 | NP_660142:NM_145159 | EGF | (311-344) |
| HG1014714 | NP_660142:NM_145159 | EGF | (351-382) |
| HG1014714 | NP_660142:NM_145159 | EGF | (389-420) |
| HG1014714 | NP_660142:NM_145159 | EGF | (427-457) |
| HG1014714 | NP_660142:NM_145159 | EGF | (464-495) |
| HG1014714 | NP_660142:NM_145159 | EGF | (502-533) |
| HG1014714 | NP_660142:NM_145159 | EGF | (602-633) |
| HG1014714 | NP_660142:NM_145159 | EGF | (640-671) |
| HG1014714 | NP_660142:NM_145159 | EGF | (678-709) |
| HG1014714 | NP_660142:NM_145159 | EGF | (717-748) |
| HG1014714 | NP_660142:NM_145159 | EGF | (755-786) |
| HG1014714 | NP_660142:NM_145159 | EGF | (793-824) |
| HG1014715 | NP_001295:NM_001304 | DUF857 | (1128-1236) |
| HG1014715 | NP_001295:NM_001304 | DUF857 | (297-406) |
| HG1014715 | NP_001295:NM_001304 | Zn_carbOpept | (501-684) |
| HG1014715 | NP_001295:NM_001304 | Zn_carbOpept | (56-270) |
| HG1014715 | NP_001295:NM_001304 | DUF857 | (709-818) |
| HG1014715 | NP_001295:NM_001304 | Zn_carbOpept | (931-1109) |
| HG1014716 | NP_680477:NM_148172 | PEMT | (39-236) |
| HG1014717 | NP_680478:NM_148173 | PEMT | (2-199) |
| HG1014718 | NP_054733:NM_014014 | DEAD | (146-365) |
| HG1014718 | NP_054733:NM_014014 | Sec63 | (1484-1800) |
| HG1014718 | NP_054733:NM_014014 | helicase_C | (449-535) |
| HG1014718 | NP_054733:NM_014014 | Sec63 | (653-962) |
| HG1014718 | NP_054733:NM_014014 | DEAD | (993-1203) |
| HG1014719 | NP_803545:NM_177526 | PAP2 | (51-192) |
| HG1014720 | NP_808211:NM_177543 | PAP2 | (128-269) |
| HG1014721 | NP_003771:NM_003780 | Galactosyl_T_2 | (97-366) |
| HG1014722 | NP_000079:NM_000088 | Collagen | (1020-1078) |
| HG1014722 | NP_000079:NM_000088 | Collagen | (1079-1138) |
| HG1014722 | NP_000079:NM_000088 | Collagen | (109-158) |
| HG1014722 | NP_000079:NM_000088 | Collagen | (1139-1192) |
| HG1014722 | NP_000079:NM_000088 | COLFI | (1245-1463) |
| HG1014722 | NP_000079:NM_000088 | Collagen | (177-235) |
| HG1014722 | NP_000079:NM_000088 | Collagen | (236-295) |
| HG1014722 | NP_000079:NM_000088 | Collagen | (296-355) |
| HG1014722 | NP_000079:NM_000088 | Collagen | (356-415) |
| HG1014722 | NP_000079:NM_000088 | Collagen | (416-475) |
| HG1014722 | NP_000079:NM_000088 | Collagen | (476-535) |
| HG1014722 | NP_000079:NM_000088 | Collagen | (536-595) |
| HG1014722 | NP_000079:NM_000088 | Collagen | (596-655) |
| HG1014722 | NP_000079:NM_000088 | Collagen | (656-715) |
| HG1014722 | NP_000079:NM_000088 | Collagen | (716-775) |
| HG1014722 | NP_000079:NM_000088 | Collagen | (779-838) |
| HG1014722 | NP_000079:NM_000088 | Collagen | (839-898) |
| HG1014722 | NP_000079:NM_000088 | Collagen | (899-958) |
| HG1014722 | NP_000079:NM_000088 | Collagen | (959-1018) |
| HG1014723 | NP_001533:NM_001542 | ig | (160-248) |
| HG1014723 | NP_001533:NM_001542 | ig | (295-378) |
| HG1014723 | NP_001533:NM_001542 | ig | (35-122) |
| HG1014723 | NP_001533:NM_001542 | ig | (445-533) |
| HG1014723 | NP_001533:NM_001542 | ig | (714-804) |
| HG1014724 | NP_001238:NM_001247 | GDA1_CD39 | (430-480) |
| HG1014724 | NP_001238:NM_001247 | GDA1_CD39 | (93-332) |
| HG1014725 | NP_004952:NM_004961 | Neur_chan_memb | (284-379) |
| HG1014725 | NP_004952:NM_004961 | Neur_chan_memb | (475-500) |
| HG1014725 | NP_004952:NM_004961 | Neur_chan_LBD | (71-277) |
| HG1014726 | NP_038464:NM_013436 | no_pfam | |
| HG1014727 | NP_054644:NM_013989 | T4_deiodinase | (4-262) |
| HG1014728 | NP_054699:NM_013993 | F5_F8_type_C | (34-182) |
| HG1014728 | NP_054699:NM_013993 | pkinase | (610-905) |
| HG1014729 | NP_054700:NM_013994 | F5_F8_type_C | (34-182) |
| HG1014729 | NP_054700:NM_013994 | pkinase | (610-911) |
| HG1014730 | NP_057311:NM_016227 | no_pfam | |
| HG1014731 | NP_057725:NM_016641 | GDPD | (70-327) |
| HG1014732 | NP_005680:NM_005689 | ABC_membrane | (265-544) |
| HG1014732 | NP_005680:NM_005689 | ABC_tran | (616-800) |
| HG1014733 | NP_003777:NM_003786 | ABC_tran | (1316-1499) |
| HG1014733 | NP_003777:NM_003786 | ABC_membrane | (311-582) |
| HG1014733 | NP_003777:NM_003786 | ABC_tran | (654-827) |
| HG1014733 | NP_003777:NM_003786 | ABC_membrane | (971-1244) |
| HG1014734 | NP_064421:NM_020037 | ABC_membrane | (311-582) |
| HG1014734 | NP_064421:NM_020037 | ABC_tran | (654-827) |
| HG1014734 | NP_064421:NM_020037 | ABC_membrane | (971-1193) |
| HG1014735 | 10047349:10047348 | ank | (854-886) |
| HG1014735 | 10047349:10047348 | ank | (887-910) |
| HG1014736 | 10435899:10435898 | DEAD | (146-365) |
| HG1014736 | 10435899:10435898 | helicase_C | (451-535) |
| HG1014736 | 10435899:10435898 | Sec63 | (653-771) |
| HG1014737 | 10438061:10438060 | no_pfam | |
| HG1014738 | 10443048:4826835 | Peptidase_M10 | (109-215) |
| HG1014738 | 10443048:4826835 | fn2 | (230-271) |
| HG1014738 | 10443048:4826835 | Peptidase_M10_N | (26-103) |
| HG1014738 | 10443048:4826835 | fn2 | (288-329) |
| HG1014738 | 10443048:4826835 | fn2 | (347-388) |
| HG1014738 | 10443048:4826835 | PT | (472-507) |
| HG1014738 | 10443048:4826835 | hemopexin | (521-565) |
| HG1014738 | 10443048:4826835 | hemopexin | (567-608) |
| HG1014738 | 10443048:4826835 | hemopexin | (613-659) |
| HG1014738 | 10443048:4826835 | hemopexin | (661-704) |
| HG1014739 | 10863065:10863064 | T4_deiodinase | (111-298) |
| HG1014739 | 10863065:10863064 | T4_deiodinase | (4-74) |
| HG1014740 | 10863067:10863066 | T4_deiodinase | (1-21) |
| HG1014741 | 11245444:11245443 | ABC_membrane | (319-598) |
| HG1014741 | 11245444:11245443 | ABC_tran | (670-854) |
| HG1014742 | 11245446:11245443 | no_pfam | |
| HG1014743 | 12082644:12082643 | Beach | (1-101) |
| HG1014743 | 12082644:12082643 | WD40 | (241-282) |
| HG1014744 | 12275809:12275808 | Galactosyl_T_2 | (77-344) |
| HG1014745 | 12314010:24797104 | fn3 | (1-85) |
| HG1014745 | 12314010:24797104 | pkinase | (187-446) |
| HG1014745 | 12314010:24797104 | SAM | (477-541) |
| HG1014746 | 12314011:17975764 | fn3 | (1-85) |
| HG1014746 | 12314011:17975764 | pkinase | (187-446) |
| HG1014746 | 12314011:17975764 | SAM | (477-541) |
| HG1014747 | 12653567:12653566 | PEMT | (39-236) |
| HG1014748 | 12697587:12697586 | T4_deiodinase | (4-74) |
| HG1014749 | 12803155:12803154 | no_pfam | |
| HG1014750 | 12803915:12803914 | Glyco_hydro_63 | (1-562) |
| HG1014751 | 13279206:13279205 | ALG3 | (40-401) |
| HG1014752 | 13325454:13325453 | Galactosyl_T_2 | (77-344) |
| HG1014753 | 13517342:7705321 | no_pfam | |
| HG1014754 | 13517410:7705321 | no_pfam | |
| HG1014755 | 13898643:13898642 | no_pfam | |
| HG1014756 | 13898645:13898644 | no_pfam | |
| HG1014757 | 14043169:14043168 | no_pfam | |
| HG1014758 | 14043179:14043178 | Sec63 | (150-459) |
| HG1014758 | 14043179:14043178 | DEAD | (490-700) |
| HG1014758 | 14043179:14043178 | Sec63 | (981-1297) |
| HG1014759 | 14043430:14043429 | Kunitz_BPTI | (133-183) |
| HG1014759 | 14043430:14043429 | Kunitz_BPTI | (38-88) |
| HG1014760 | 14249879:14243878 | Zip | (1-167) |
| HG1014760 | 14249879:14249878 | Zip | (181-370) |
| HG1014761 | 14250593:14250592 | Calpain_III | (365-522) |
| HG1014761 | 14250593:14250592 | Peptidase_C2 | (55-354) |
| HG1014761 | 14250593:14250592 | efhand | (619-647) |
| HG1014762 | 14550482:14550481 | no_pfam | |
| HG1014763 | 14602901:14602900 | no_pfam | |
| HG1014764 | 14724070:22042187 | no_pfam | |
| HG1014765 | 14726864:14726863 | no_pfam | |
| HG1014766 | 15029376:15029375 | SK_channel | (12-121) |
| HG1014766 | 15029376:15029375 | CaMBD | (304-377) |
| HG1014767 | 15214801:15214800 | no_pfam | |
| HG1014768 | 15214917:15214916 | SNARE | (31-78) |
| HG1014769 | 15559191:9955969 | ABC_tran | (1316-1486) |
| HG1014769 | 15559191:9955969 | ABC_membrane | (311-582) |
| HG1014769 | 15559191:9955969 | ABC_tran | (654-827) |
| HG1014769 | 15559191:9955969 | ABC_membrane | (971-1244) |
| HG1014770 | 15680237:15680236 | ig | (160-217) |
| HG1014770 | 15680237:15680236 | ig | (252-301) |
| HG1014770 | 15680237:15680236 | ig | (341-398) |
| HG1014771 | 15779135:15779134 | PDZ | (209-297) |
| HG1014771 | 15779135:15779134 | PDZ | (305-393) |
| HG1014771 | 15779135:15779134 | PDZ | (76-154) |
| HG1014772 | 15929829:15929828 | no_pfam | |
| HG1014773 | 1632766:1632765 | zf-C3HC4 | (1647-1686) |
| HG1014774 | 16552593:16552592 | ABC_membrane | (189-468) |
| HG1014774 | 16552593:16552592 | ABC_tran | (540-724) |
| HG1014775 | 1688260:4505206 | hemopexin | (102-145) |
| HG1014775 | 1688260:4505206 | hemopexin | (150-197) |
| HG1014775 | 1688260:4505206 | hemopexin | (199-240) |
| HG1014775 | 1688260:4505206 | hemopexin | (58-100) |
| HG1014776 | 1747371:1747370 | Neur_chan_memb | (283-378) |
| HG1014776 | 1747371:1747370 | Neur_chan_memb | (474-499) |
| HG1014776 | 1747371:1747370 | Neur_chan_LBD | (71-276) |
| HG1014777 | 179629:179624 | Collagen | (1-51) |
| HG1014778 | 179630:22328091 | Collagen | (3-38) |
| HG1014779 | 179631:179626 | Collagen | (1-60) |
| HG1014780 | 18027796:18027795 | no_pfam | |
| HG1014781 | 18044628:18044627 | PI-PLC-Y | (1-40) |
| HG1014781 | 18044628:18044627 | C2 | (60-152) |
| HG1014782 | 18676646:18676645 | C2 | (205-297) |
| HG1014782 | 18676646:18676645 | PI-PLC-Y | (70-185) |
| HG1014783 | 1888409:22328091 | Collagen | (1019-1069) |
| HG1014783 | 1888409:22328091 | Collagen | (109-158) |
| HG1014783 | 1888409:22328091 | Collagen | (177-235) |
| HG1014783 | 1888409:22328091 | Collagen | (236-295) |
| HG1014783 | 1888409:22328091 | Collagen | (296-355) |
| HG1014783 | 1888409:22328091 | Collagen | (356-415) |
| HG1014783 | 1888409:22328091 | Collagen | (416-475) |
| HG1014783 | 1888409:22328091 | Collagen | (476-535) |
| HG1014783 | 1888409:22328091 | Collagen | (536-595) |
| HG1014783 | 1888409:22328091 | Collagen | (596-655) |
| HG1014783 | 1888409:22328091 | Collagen | (656-715) |
| HG1014783 | 1888409:22328091 | Collagen | (716-775) |
| HG1014783 | 1888409:22328091 | Collagen | (779-838) |
| HG1014783 | 1888409:22328091 | Collagen | (839-898) |
| HG1014783 | 1888409:22328091 | Collagen | (899-958) |
| HG1014783 | 1888409:22328091 | Collagen | (959-1018) |
| HG1014784 | 19684107:19684106 | GDA1_CD39 | (429-479) |
| HG1014784 | 19684107:19684106 | GDA1_CD39 | (92-331) |
| HG1014785 | 19913138:20130436 | Glyco_hydro_63 | (50-837) |
| HG1014786 | 20521698:20521697 | Dopey_N | (1-163) |
| HG1014787 | 20540895:20540894 | no_pfam | |
| HG1014788 | 20541809:20541808 | no_pfam | |
| HG1014789 | 21104416:21104415 | DDOST_48 kD | (9-438) |
| HG1014790 | 21434741:21434740 | Beach | (2201-2478) |
| HG1014790 | 21434741:21434740 | WD40 | (2618-2659) |
| HG1014791 | 21706696:21706695 | cadherin | (159-248) |
| HG1014792 | 21739637:21739636 | GDA1_CD39 | (112-351) |
| HG1014792 | 21739637:21739636 | GDA1_CD39 | (449-499) |
| HG1014793 | 21748877:21748876 | DEAD | (471-567) |
| HG1014793 | 21748877:21748876 | Sec63 | (561-614) |
| HG1014794 | 21750497:21750496 | AIRS_C | (194-369) |
| HG1014794 | 21750497:21750496 | AIRS | (66-190) |
| HG1014795 | 21752841:21752840 | AIRS_C | (186-361) |
| HG1014795 | 21752841:21752840 | AIRS | (58-182) |
| HG1014796 | 21757691:21757690 | ig | (367-457) |
| HG1014796 | 21757691:21757690 | ig | (504-593) |
| HG1014796 | 21757691:21757690 | ig | (98-186) |
| HG1014797 | 21929079:19923767 | GPS | (342-394) |
| HG1014797 | 21929079:19923767 | 7tm_2 | (400-665) |
| HG1014798 | 219495:219494 | ig | (160-217) |
| HG1014798 | 219495:219494 | ig | (252-301) |
| HG1014799 | 21961497:21961496 | no_pfam | |
| HG1014800 | 2197067:2197066 | DSL | (178-240) |
| HG1014800 | 2197067:2197066 | EGF | (311-344) |
| HG1014800 | 2197067:2197066 | EGF | (351-382) |
| HG1014800 | 2197067:2197066 | EGF | (389-420) |
| HG1014800 | 2197067:2197066 | EGF | (427-458) |
| HG1014800 | 2197067:2197066 | EGF | (465-495) |
| HG1014800 | 2197067:2197066 | EGF | (502-533) |
| HG1014800 | 2197067:2197066 | EGF | (540-571) |
| HG1014800 | 2197067:2197066 | EGF | (640-671) |
| HG1014800 | 2197067:2197066 | EGF | (678-709) |
| HG1014800 | 2197067:2197066 | EGF | (716-747) |
| HG1014800 | 2197067:2197066 | EGF | (755-786) |
| HG1014800 | 2197067:2197066 | EGF | (793-824) |
| HG1014800 | 2197067:2197066 | EGF | (831-862) |
| HG1014801 | 22044017:22044016 | no_pfam | |
| HG1014802 | 22328092:22328091 | Collagen | (1019-1078) |
| HG1014802 | 22328092:22328091 | Collagen | (1079-1138) |
| HG1014802 | 22328092:22328091 | Collagen | (109-158) |
| HG1014802 | 22328092:22328091 | Collagen | (1139-1192) |
| HG1014802 | 22328092:22328091 | COLFI | (1245-1463) |
| HG1014802 | 22328092:22328091 | Collagen | (177-235) |
| HG1014802 | 22328092:22328091 | Collagen | (236-295) |
| HG1014802 | 22328092:22328091 | Collagen | (296-355) |
| HG1014802 | 22328092:22328091 | Collagen | (356-415) |
| HG1014802 | 22328092:22328091 | Collagen | (416-475) |
| HG1014802 | 22328092:22328091 | Collagen | (476-535) |
| HG1014802 | 22328092:22328091 | Collagen | (536-595) |
| HG1014802 | 22328092:22328091 | Collagen | (596-655) |
| HG1014802 | 22328092:22328091 | Collagen | (656-715) |
| HG1014802 | 22328092:22328091 | Collagen | (719-755) |
| HG1014802 | 22328092:22328091 | Collagen | (779-838) |
| HG1014802 | 22328092:22328091 | Collagen | (839-898) |
| HG1014802 | 22328092:22328091 | Collagen | (899-958) |
| HG1014802 | 22328092:22328091 | Collagen | (959-1018) |
| HG1014803 | 22532481:4826835 | Peptidase_M10 | (109-215) |
| HG1014803 | 22532481:4826835 | fn2 | (230-271) |
| HG1014803 | 22532481:4826835 | Peptidase_M10_N | (26-103) |
| HG1014803 | 22532481:4826835 | fn2 | (288-329) |
| HG1014803 | 22532481:4826835 | fn2 | (347-388) |
| HG1014803 | 22532481:4826835 | PT | (472-507) |
| HG1014803 | 22532481:4826835 | hemopexin | (521-565) |
| HG1014803 | 22532481:4826835 | hemopexin | (567-608) |
| HG1014803 | 22532481:4826835 | hemopexin | (613-659) |
| HG1014803 | 22532481:4826835 | hemopexin | (661-704) |
| HG1014804 | 2270923:33910 | fn3 | (1127-1208) |
| HG1014804 | 2270923:33910 | fn3 | (1220-1310) |
| HG1014804 | 2270923:33910 | fn3 | (1458-1542) |
| HG1014804 | 2270923:33910 | fn3 | (1571-1658) |
| HG1014804 | 2270923:33910 | integrin_B | (37-455) |
| HG1014804 | 2270923:33910 | Calx-beta | (979-1084) |
| HG1014805 | 2270924:21361206 | fn3 | (1127-1208) |
| HG1014805 | 2270924:21361206 | fn3 | (1220-1310) |
| HG1014805 | 2270924:21361206 | fn3 | (1528-1612) |
| HG1014805 | 2270924:21361206 | fn3 | (1641-1728) |
| HG1014805 | 2270924:21361206 | integrin_B | (37-455) |
| HG1014805 | 2270924:21361206 | Calx-beta | (979-1084) |
| HG1014806 | 2270925:33956 | fn3 | (1127-1208) |
| HG1014806 | 2270925:33956 | fn3 | (1220-1310) |
| HG1014806 | 2270925:33956 | fn3 | (1511-1595) |
| HG1014806 | 2270925:33956 | fn3 | (1624-1711) |
| HG1014806 | 2270925:33956 | integrin_B | (37-455) |
| HG1014806 | 2270925:33956 | Calx-beta | (979-1084) |
| HG1014807 | 2285958:2285960 | Neur_chan_memb | (284-379) |
| HG1014807 | 2285958:2285960 | Neur_chan_memb | (475-500) |
| HG1014807 | 2285958:2285960 | Neur_chan_LBD | (71-277) |
| HG1014808 | 2293523:21361206 | no_pfam | |
| HG1014809 | 239158:239157 | integrin_A | (112-126) |
| HG1014810 | 2432002:2432001 | DSL | (178-240) |
| HG1014810 | 2432002:2432001 | EGF | (311-344) |
| HG1014810 | 2432002:2432001 | EGF | (351-382) |
| HG1014810 | 2432002:2432001 | EGF | (389-420) |
| HG1014810 | 2432002:2432001 | EGF | (427-458) |
| HG1014810 | 2432002:2432001 | EGF | (465-495) |
| HG1014810 | 2432002:2432001 | EGF | (502-533) |
| HG1014810 | 2432002:2432001 | EGF | (540-571) |
| HG1014810 | 2432002:2432001 | EGF | (640-671) |
| HG1014810 | 2432002:2432001 | EGF | (678-709) |
| HG1014810 | 2432002:2432001 | EGF | (716-747) |
| HG1014810 | 2432002:2432001 | EGF | (755-786) |
| HG1014810 | 2432002:2432001 | EGF | (793-824) |
| HG1014810 | 2432002:2432001 | EGF | (831-862) |
| HG1014811 | 24496473:24496472 | no_pfam | |
| HG1014812 | 24658543:24658542 | I_LWEQ | (252-445) |
| HG1014813 | 24659964:24659963 | Zip | (279-431) |
| HG1014813 | 24659964:24659963 | Zip | (47-265) |
| HG1014814 | 2598968:2598967 | Kunitz_BPTI | (133-183) |
| HG1014814 | 2598968:2598967 | Kunitz_BPTI | (38-88) |
| HG1014815 | 2605947:2605946 | DSL | (178-240) |
| HG1014815 | 2605947:2605946 | EGF | (311-344) |
| HG1014815 | 2605947:2605946 | EGF | (351-382) |
| HG1014815 | 2605947:2605946 | EGF | (389-420) |
| HG1014815 | 2605947:2605946 | EGF | (427-457) |
| HG1014815 | 2605947:2605946 | EGF | (464-495) |
| HG1014815 | 2605947:2605946 | EGF | (502-533) |
| HG1014815 | 2605947:2605946 | EGF | (602-633) |
| HG1014815 | 2605947:2605946 | EGF | (640-671) |
| HG1014815 | 2605947:2605946 | EGF | (678-709) |
| HG1014815 | 2605947:2605946 | EGF | (717-748) |
| HG1014815 | 2605947:2605946 | EGF | (755-786) |
| HG1014815 | 2605947:2605946 | EGF | (793-824) |
| HG1014816 | 2662364:2687860 | zf-C3HC4 | (1873-1912) |
| HG1014817 | 2662375:473936 | DDOST_48 kD | (26-455) |
| HG1014818 | 27477822:27477821 | no_pfam | |
| HG1014819 | 27480564:27480563 | no_pfam | |
| HG1014820 | 27499509:27499508 | ENTH | (28-147) |
| HG1014820 | 27499509:27499508 | I_LWEQ | (819-1012) |
| HG1014821 | 27529860:27529859 | no_pfam | |
| HG1014822 | 2765402:2765401 | DSL | (162-224) |
| HG1014822 | 2765402:2765401 | EGF | (295-328) |
| HG1014822 | 2765402:2765401 | EGF | (335-366) |
| HG1014822 | 2765402:2765401 | EGF | (374-405) |
| HG1014822 | 2765402:2765401 | EGF | (412-443) |
| HG1014822 | 2765402:2765401 | EGF | (450-480) |
| HG1014822 | 2765402:2765401 | EGF | (487-518) |
| HG1014822 | 2765402:2765401 | EGF | (525-556) |
| HG1014822 | 2765402:2765401 | EGF | (625-656) |
| HG1014822 | 2765402:2765401 | EGF | (663-694) |
| HG1014822 | 2765402:2765401 | EGF | (701-732) |
| HG1014822 | 2765402:2765401 | EGF | (740-771) |
| HG1014822 | 2765402:2765401 | EGF | (778-809) |
| HG1014822 | 2765402:2765401 | EGF | (816-847) |
| HG1014823 | 27694125:27694124 | PI-PLC-X | (185-330) |
| HG1014823 | 27694125:27694124 | efhand | (31-59) |
| HG1014823 | 27694125:27694124 | PI-PLC-Y | (483-563) |
| HG1014823 | 27694125:27694124 | C2 | (582-674) |
| HG1014824 | 28175817:28175816 | no_pfam | |
| HG1014825 | 28207917:28207916 | Cornichon | (4-85) |
| HG1014826 | 28273134:28273133 | PI-PLC-X | (245-390) |
| HG1014826 | 28273134:28273133 | PI-PLC-Y | (543-658) |
| HG1014826 | 28273134:28273133 | C2 | (678-770) |
| HG1014826 | 28273134:28273133 | efhand | (91-119) |
| HG1014827 | 28273138:28273137 | C2 | (172-241) |
| HG1014828 | 28277412:28277411 | no_pfam | |
| HG1014829 | 28279793:28279792 | ABC_membrane | (337-572) |
| HG1014830 | 28374245:28374244 | DUF857 | (1128-1236) |
| HG1014830 | 28374245:28374244 | DUF857 | (297-406) |
| HG1014830 | 28374245:28374244 | Zn_carbOpept | (501-684) |
| HG1014830 | 28374245:28374244 | Zn_carbOpept | (56-270) |
| HG1014830 | 28374245:28374244 | DUF857 | (709-818) |
| HG1014830 | 28374245:28374244 | Zn_carbOpept | (931-1109) |
| HG1014831 | 285917:285916 | pkinase | (11-241) |
| HG1014831 | 285917:285916 | SAM | (272-336) |
| HG1014832 | 28981412:28981411 | Y_phosphatase | (1366-1597) |
| HG1014832 | 28981412:28981411 | ig | (149-209) |
| HG1014832 | 28981412:28981411 | Y_phosphatase | (1655-1888) |
| HG1014832 | 28981412:28981411 | ig | (246-300) |
| HG1014832 | 28981412:28981411 | fn3 | (319-401) |
| HG1014832 | 28981412:28981411 | fn3 | (413-500) |
| HG1014832 | 28981412:28981411 | ig | (47-109) |
| HG1014832 | 28981412:28981411 | fn3 | (512-594) |
| HG1014832 | 28981412:28981411 | fn3 | (606-696) |
| HG1014832 | 28981412:28981411 | fn3 | (708-800) |
| HG1014832 | 28981412:28981411 | fn3 | (812-895) |
| HG1014832 | 28981412:28981411 | fn3 | (906-991) |
| HG1014833 | 2924620:2924619 | Kunitz_BPTI | (133-183) |
| HG1014833 | 2924620:2924619 | Kunitz_BPTI | (38-88) |
| HG1014834 | 2951948:7637876 | GDPD | (1-115) |
| HG1014835 | 30016:30015 | Collagen | (109-158) |
| HG1014835 | 30016:30015 | Collagen | (177-235) |
| HG1014835 | 30016:30015 | Collagen | (236-295) |
| HG1014835 | 30016:30015 | Collagen | (296-355) |
| HG1014835 | 30016:30015 | Collagen | (356-415) |
| HG1014835 | 30016:30015 | vwc | (40-95) |
| HG1014835 | 30016:30015 | Collagen | (416-471) |
| HG1014836 | 31223:31222 | pkinase | (1-61) |
| HG1014837 | 3132270:3132269 | ABC_tran | (1316-1499) |
| HG1014837 | 3132270:3132269 | ABC_membrane | (311-582) |
| HG1014837 | 3132270:3132269 | ABC_tran | (654-827) |
| HG1014837 | 3132270:3132269 | ABC_membrane | (971-1244) |
| HG1014838 | 3172147:219494 | ig | (160-217) |
| HG1014838 | 3172147:219494 | ig | (252-301) |
| HG1014839 | 33911:33910 | fn3 | (1127-1208) |
| HG1014839 | 33911:33910 | fn3 | (1220-1310) |
| HG1014839 | 33911:33910 | fn3 | (1458-1542) |
| HG1014839 | 33911:33910 | fn3 | (1571-1658) |
| HG1014839 | 33911:33910 | integrin_B | (37-455) |
| HG1014839 | 33911:33910 | Calx-beta | (979-1084) |
| HG1014840 | 33942:33941 | integrin_A | (1032-1046) |
| HG1014840 | 33942:33941 | FG-GAP | (310-361) |
| HG1014840 | 33942:33941 | FG-GAP | (372-416) |
| HG1014840 | 33942:33941 | FG-GAP | (426-468) |
| HG1014841 | 33957:33956 | fn3 | (1127-1208) |
| HG1014841 | 33957:33956 | fn3 | (1220-1310) |
| HG1014841 | 33957:33956 | fn3 | (1511-1595) |
| HG1014841 | 33957:33956 | fn3 | (1624-1711) |
| HG1014841 | 33957:33956 | integrin_B | (37-455) |
| HG1014841 | 33957:33956 | Calx-beta | (979-1084) |
| HG1014842 | 35658:35657 | Collagen | (112-157) |
| HG1014842 | 35658:35657 | vwc | (40-95) |
| HG1014843 | 3582767:3582766 | CaMBD | (78-141) |
| HG1014844 | 37200:37199 | ig | (160-217) |
| HG1014844 | 37200:37199 | ig | (252-301) |
| HG1014845 | 37204:37203 | ig | (161-210) |
| HG1014845 | 37204:37203 | ig | (250-307) |
| HG1014845 | 37204:37203 | ig | (69-126) |
| HG1014846 | 3721836:3721835 | I_LWEQ | (641-834) |
| HG1014847 | 3721898:12804512 | JTB | (1-94) |
| HG1014848 | 407590:407589 | COLFI | (67-283) |
| HG1014849 | 4102188:4102187 | ABC_tran | (1317-1500) |
| HG1014849 | 4102188:4102187 | ABC_membrane | (311-583) |
| HG1014849 | 4102188:4102187 | ABC_tran | (655-828) |
| HG1014849 | 4102188:4102187 | ABC_membrane | (972-1245) |
| HG1014850 | 4587083:4587082 | ABC_tran | (1220-1403) |
| HG1014850 | 4587083:4587082 | ABC_membrane | (179-447) |
| HG1014850 | 4587083:4587082 | ABC_tran | (588-759) |
| HG1014850 | 4587083:4587082 | ABC_membrane | (860-1147) |
| HG1014851 | 4755085:14719826 | Collagen | (1016-1075) |
| HG1014851 | 4755085:14719826 | Collagen | (1076-1135) |
| HG1014851 | 4755085:14719826 | Collagen | (109-150) |
| HG1014851 | 4755085:14719826 | Collagen | (1136-1189) |
| HG1014851 | 4755085:14719826 | COLFI | (1242-1460) |
| HG1014851 | 4755085:14719826 | Collagen | (174-232) |
| HG1014851 | 4755085:14719826 | Collagen | (233-292) |
| HG1014851 | 4755085:14719826 | Collagen | (293-352) |
| HG1014851 | 4755085:14719826 | Collagen | (353-412) |
| HG1014851 | 4755085:14719826 | Collagen | (413-472) |
| HG1014851 | 4755085:14719826 | Collagen | (473-532) |
| HG1014851 | 4755085:14719826 | Collagen | (533-592) |
| HG1014851 | 4755085:14719826 | Collagen | (593-652) |
| HG1014851 | 4755085:14719826 | Collagen | (653-712) |
| HG1014851 | 4755085:14719826 | Collagen | (713-772) |
| HG1014851 | 4755085:14719826 | Collagen | (776-835) |
| HG1014851 | 4755085:14719826 | Collagen | (836-895) |
| HG1014851 | 4755085:14719826 | Collagen | (896-955) |
| HG1014851 | 4755085:14719826 | Collagen | (956-1015) |
| HG1014852 | 4826563:4826562 | ABC_tran | (1316-1499) |
| HG1014852 | 4826563:4826562 | ABC_membrane | (311-582) |
| HG1014852 | 4826563:4826562 | ABC_tran | (654-827) |
| HG1014852 | 4826563:4826562 | ABC_membrane | (971-1244) |
| HG1014853 | 4836765:4836764 | GPS | (342-394) |
| HG1014853 | 4836765:4836764 | 7tm_2 | (400-665) |
| HG1014854 | 4894209:4894208 | Cornichon | (1-126) |
| HG1014855 | 495678:495677 | EPH_lbd | (15-191) |
| HG1014855 | 495678:495677 | fn3 | (319-415) |
| HG1014855 | 495678:495677 | fn3 | (430-515) |
| HG1014855 | 495678:495677 | pkinase | (616-875) |
| HG1014855 | 495678:495677 | SAM | (906-970) |
| HG1014856 | 5002294:4826835 | Peptidase_M10_N | (26-79) |
| HG1014857 | 5006891:5006890 | ABC_tran | (1220-1403) |
| HG1014857 | 5006891:5006890 | ABC_membrane | (179-447) |
| HG1014857 | 5006891:5006890 | ABC_tran | (588-759) |
| HG1014857 | 5006891:5006890 | ABC_membrane | (860-1147) |
| HG1014858 | 5031476:5031475 | ABC_tran | (74-257) |
| HG1014859 | 5114047:5114046 | Sec63 | (268-584) |
| HG1014860 | 5726563:4557674 | integrin_A | (1038-1052) |
| HG1014860 | 5726563:4557674 | FG-GAP | (316-367) |
| HG1014860 | 5726563:4557674 | FG-GAP | (378-422) |
| HG1014860 | 5726563:4557674 | FG-GAP | (432-474) |
| HG1014861 | 5851985:15488900 | zf-C3HC4 | (16-56) |
| HG1014861 | 5851985:15488900 | zf-B_box | (93-132) |
| HG1014862 | 606777:29447 | no_pfam | |
| HG1014863 | 6941892:6941891 | zf-C3HC4 | (16-44) |
| HG1014864 | 7022121:7022120 | ank | (101-124) |
| HG1014864 | 7022121:7022120 | ank | (68-100) |
| HG1014865 | 7106834:7106833 | JTB | (10-117) |
| HG1014866 | 7159057:7159056 | T4_deiodinase | (4-115) |
| HG1014867 | 762938:30092 | COLFI | (17-226) |
| HG1014868 | 7768766:4826652 | Dopey_N | (2-314) |
| HG1014869 | 7770185:7770184 | Sec63 | (2-318) |
| HG1014870 | proteinkinase320A:proteinkinase320B | F5_F8_type_C | (34-182) |
| HG1014870 | proteinkinase320A:proteinkinase320B | pkinase | (572-867) |
| HG1014871 | 307091:186775 | thyroglobulin_1 | (66-135) |
| HG1014872 | 31417919:12803236 | Galactosyl_T_2 | (53-300) |
| HG1014873 | 1160925:1160924 | F5_F8_type_C | (34-182) |
| HG1014873 | 1160925:1160924 | pkinase | (573-868) |
| HG1014874 | 179435:179434 | ig | (160-217) |
| HG1014874 | 179435:179434 | ig | (252-301) |
| HG1014875 | 219497:219496 | ig | (160-217) |
| HG1014875 | 219497:219496 | ig | (252-301) |
| HG1014875 | 219497:219496 | ig | (341-398) |
| HG1014876 | 2554610:2554609 | ABC_membrane | (369-656) |
| HG1014876 | 2554610:2554609 | ABC_tran | (729-912) |
| HG1014876 | 2554610:2554609 | ABC_tran | (97-268) |
| HG1014877 | 29387396:29387395 | ig | (149-215) |
| HG1014877 | 29387396:29387395 | ig | (47-109) |
| HG1014878 | 29421204:29421203 | ank | (1006-1038) |
| HG1014878 | 29421204:29421203 | ank | (1039-1071) |
| HG1014878 | 29421204:29421203 | ank | (791-823) |
| HG1014878 | 29421204:29421203 | ank | (824-847) |
| HG1014878 | 29421204:29421203 | ank | (907-939) |
| HG1014878 | 29421204:29421203 | ank | (940-972) |
| HG1014878 | 29421204:29421203 | ank | (973-1005) |
| HG1014879 | 29476766:29476765 | no_pfam | |
| HG1014880 | 29792320:29792319 | no_pfam | |
| HG1014881 | 30046456:30046455 | ABC_membrane | (312-547) |
| HG1014882 | 30046796:30046795 | FG-GAP | (60-102) |
| HG1014882 | 30046796:30046795 | FG-GAP | (6-50) |
| HG1014882 | 30046796:30046795 | integrin_A | (651-665) |
| HG1014883 | 30313820:30313819 | ABC_tran | (1177-1360) |
| HG1014883 | 30313820:30313819 | ABC_membrane | (179-447) |
| HG1014883 | 30313820:30313819 | ABC_tran | (588-759) |
| HG1014883 | 30313820:30313819 | ABC_membrane | (860-1007) |
| HG1014884 | 31323051:31323050 | Kunitz_BPTI | (250-300) |
| HG1014884 | 31323051:31323050 | ldl_recept_a | (333-371) |
| HG1014884 | 31323051:31323050 | Kunitz_BPTI | (391-441) |
| HG1014885 | 31873230:31873229 | fn3 | (105-188) |
| HG1014885 | 31873230:31873229 | fn3 | (1-93) |
| HG1014885 | 31873230:31873229 | fn3 | (199-284) |
| HG1014885 | 31873230:31873229 | Y_phosphatase | (659-890) |
| HG1014885 | 31873230:31873229 | Y_phosphatase | (948-1181) |
| HG1014886 | 32812254:32812253 | PDZ | (1071-1159) |
| HG1014886 | 32812254:32812253 | PDZ | (699-785) |
| HG1014886 | 32812254:32812253 | PDZ | (842-920) |
| HG1014886 | 32812254:32812253 | PDZ | (975-1063) |
| HG1014887 | 32966069:32966068 | GDA1_CD39 | (430-480) |
| HG1014887 | 32966069:32966068 | GDA1_CD39 | (93-332) |
| HG1014888 | 5825553:5825552 | PEMT | (2-199) |
| HG1014889 | 11282038:6808452 | Galactosyl_T_2 | (1-218) |
| HG1014890 | 20138797:2605944 | DSL | (178-240) |
| HG1014890 | 20138797:2605944 | EGF | (311-344) |
| HG1014890 | 20138797:2605944 | EGF | (351-382) |
| HG1014890 | 20138797:2605944 | EGF | (389-420) |
| HG1014890 | 20138797:2605944 | EGF | (427-458) |
| HG1014890 | 20138797:2605944 | EGF | (465-495) |
| HG1014890 | 20138797:2605944 | EGF | (502-533) |
| HG1014890 | 20138797:2605944 | EGF | (540-571) |
| HG1014890 | 20138797:2605944 | EGF | (640-671) |
| HG1014890 | 20138797:2605944 | EGF | (678-709) |
| HG1014890 | 20138797:2605944 | EGF | (716-747) |
| HG1014890 | 20138797:2605944 | EGF | (755-786) |
| HG1014890 | 20138797:2605944 | EGF | (793-824) |
| HG1014890 | 20138797:2605944 | EGF | (831-862) |
| HG1014891 | 2136054:1060894 | pkinase | (113-372) |
| HG1014891 | 2136054:1060894 | SAM | (403-467) |
| HG1014892 | 2168139:6013007 | no_pfam | |
| HG1014893 | 25089854:3641620 | DUF857 | (1128-1236) |
| HG1014893 | 25089854:3641620 | DUF857 | (297-406) |
| HG1014893 | 25089854:3641620 | Zn_carbOpept | (501-684) |
| HG1014893 | 25089854:3641620 | Zn_carbOpept | (56-270) |
| HG1014893 | 25089854:3641620 | DUF857 | (709-818) |
| HG1014893 | 25089854:3641620 | Zn_carbOpept | (931-1109) |
| HG1014894 | 263064:33941 | no_pfam | |
| HG1014895 | 32425685:12655128 | Sec63 | (170-486) |
| HG1014896 | 7442652:3550323 | ABC_tran | (1316-1499) |
| HG1014896 | 7442652:3550323 | ABC_membrane | (311-582) |
| HG1014896 | 7442652:3550323 | ABC_tran | (654-827) |
| HG1014896 | 7442652:3550323 | ABC_membrane | (971-1244) |
| HG1014897 | 7459693:2293520 | integrin_B | (37-455) |
| HG1014898 | 86966:219500 | ig | (160-217) |
| HG1014898 | 86966:219500 | ig | (252-301) |
| HG1014899 | 8928547:5685863 | ABC_tran | (1220-1403) |
| HG1014899 | 8928547:5685863 | ABC_membrane | (179-447) |
| HG1014899 | 8928547:5685863 | ABC_tran | (588-759) |
| HG1014899 | 8928547:5685863 | ABC_membrane | (860-1147) |
| HG1014900 | NP_857593.1:NM_181642 | Kunitz_BPTI | (250-300) |
| HG1014900 | NP_857593.1:NM_181642 | ldl_recept_a | (333-371) |
| HG1014900 | NP_857593.1:NM_181642 | Kunitz_BPTI | (391-441) |

1-82. (canceled)

83. A method of treating cancer comprising administering a pharmaceutical composition comprising an antibody that binds PAP2C.

84. The method of claim 83, wherein the cancer is selected from lung cancer, colon cancer, bladder cancer, liver cancer, ovarian cancer, breast cancer, kidney cancer, and pancreatic cancer.

85. The method of claim 83, wherein the antibody is attached to at least one cytotoxic component.

86. The method of claim 85, wherein the at least one cytotoxic component is selected from a radioisotope, a radioactive molecule, a microbial toxin, a plant toxin, a chemotherapeutic agent, doxorubicin, and cisplatin.

87. The method of claim 83, wherein the cancer is lung cancer.

88. The method of claim 87, wherein the antibody is attached to at least one cytotoxic component.

89. The method of claim 83, wherein the cancer is colon cancer.

90. The method of claim 89, wherein the antibody is attached to at least one cytotoxic component.

91. The method of claim 83, wherein the cancer is bladder cancer.

92. The method of claim 91, wherein the antibody is attached to at least one cytotoxic component.

93. The method of claim 83, wherein the cancer is liver cancer.

94. The method of claim 93, wherein the antibody is attached to at least one cytotoxic component.

95. The method of claim 83, wherein the cancer is ovarian cancer.

96. The method of claim 95, wherein the antibody is attached to at least one cytotoxic component.

97. The method of claim 83, wherein the cancer is breast cancer.

98. The method of claim 97, wherein the antibody is attached to at least one cytotoxic component.

99. The method of claim 83, wherein the cancer is kidney cancer.

100. The method of claim 99, wherein the antibody is attached to at least one cytotoxic component.

101. The method of claim 83, wherein the cancer is pancreatic cancer.

102. The method of claim 101, wherein the antibody is attached to at least one cytotoxic component.

103. The method of any one of claims 88, 90, 92, 94, 96, 98, 100, or 102, wherein the at least one cytotoxic component is selected from a radioisotope, a radioactive molecule, a microbial toxin, a plant toxin, a chemotherapeutic agent, doxorubicin, and cisplatin.
